(19) United States
(12) Patent Application Publication
(10) Pub. No.: US 2004/0063783 A1
(43) Pub. Date: Apr. 1, 2004

(54) Nitroso diphenylamine derivatives

(76) Inventors: Claude Lardy, Lyon (FR); Jean-Yves Nioche, Limonest (FR); Lidia Caputo, Lyon (FR); Jacques Decerprit, Miribel (FR); Jean-Yves Ortholand, Saint Jean de Noist (FR); Didier Festal, Ecully (FR); Daniel Guerrier, Saint Genis Laval (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/398,238

(22) PCT Filed: Sep. 18, 2001
(86) PCT No.: PCT/EP01/10761

(51) Int. Cl. A61K 31/277; A61K 31/13

(52) U.S. Cl. 514/524; 514/645; 558/408; 564/107

(57) ABSTRACT

The present invention relates to a compound of formula (I) in which X, A, T, Y, G and R are as defined in Claim (1).

The invention relates to nitroso diphenylamine derivatives, to pharmaceutical compositions containing them and to their use for preparing medicinal products which may be used for treating pathologies characterized by an oxidative stress condition and a lack of availability of endothelial nitrogen monoxide (NO^{•}).

Nitrogen monoxide (or nitric oxide NO^{•}) is an important mediator in the physiology of cardiovascular, immune and central and peripheral nervous systems. It acts, among other mechanisms, by activation of guanylate cyclase.

Its action is ubiquitous: it is vasodilatory and gives a basal tonus to the entire vascular system. It has anti-clotting activity: its production by normal endothelial cells inhibits the formation of a thrombus. It is anti-proliferative, especially on the smooth muscle cells underlying the endothelial cells. It also inhibits the adhesion of monocytes to the vascular wall and, consequently, its conversion to a macrophage. It regulates endothelial permeability.

There is thus, in the physiological state, a situation of equilibrium between the production of free-radical species and the availability of NO.

Disequilibrium of this balance, the result of which is an excess of superoxide anions in the face of a lack of NO, leads to the development of many pathologies.

Oxidative stress is generated by many factors such as hyperglycaemia, dyslipidaemias (production of oxidized, highly atherogenic “low-density” lipoproteins (LDL)), hypoxia, insulin resistance, atherosclerosis, revascularization techniques (including angioplasties with or without a stent), chronic rejection after transplantation, the majority of inflammatory processes, and addiction to smoking. Oxidative stress is characterized at the vascular level by an increase in free radicals, in particular of superoxide anions (O₂^{•-}).

These O₂^{•-} radicals are capable of trapping the NO endogenously produced by the endothelial cells to form free-radical species that are even more deleterious, for instance peroxynitrites.

Among the pathologies concerned by a lack of production of endothelial nitrogen monoxide and/or an increase in oxidative tissue stress, mention may be made of (Recent Progress in Hormone Research (1988), 53, 43-60, table V):

atherosclerosis-associated ischaemias (lipid peroxidation, development, progress and rupture of atheroma plaques, platelet activation);

restenosis after angioplasty;

stenosis after vascular surgery;

diabetes;

insulin resistance;

retinal and renal microvascular complications of diabetes;

the cardiovascular risk of diabetes in so far as it is not explained by the conventional factors;

male erectile dysfunction;

cerebral hypoxia;

chronic rejection after organ transplantation;

articular pathologies.

The administration of active principles capable of reducing the biological activity of oxidative free-radical species (such as superoxide anions and peroxynitrites) and of increasing the content of nitrogen monoxide by a twofold mechanism: non-conversion into peroxynitrites and exogenous supply, is thus particularly desirable in the treatment of these pathologies.

The present invention provides compounds which have these two effects, antioxidant and nitrogen monoxide-donating, in the same molecule.

These compounds are capable of spontaneously generating nitrogen monoxide under physiological conditions and of trapping oxidative free radicals.

The spontaneous NO-donating effect does not induce a tachyphylactic effect, unlike compounds that are substrates of NO synthase, and unlike nitro derivatives or derivatives of oxadiazole or oxatriazole type which mobilize endogenous thiols groups to release NO.

The spontaneous NO-donating effect makes it possible to achieve pharmacological NO activity in pathologies in which the activity of NO synthase is insufficient.

More specifically, the invention relates to the compounds of the formula I:

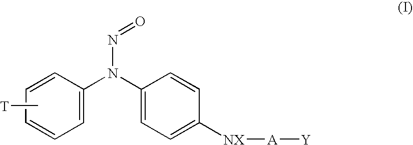

in which:

X represents a hydrogen atom; a saturated or unsaturated aliphatic hydrocarbon-based radical; or a group -A-Y;

A represents —CO—; SO₂—; —CO—NRₐ— in which the carbonyl group is linked to the nitrogen atom of NX and Rₐ represents a hydrogen atom or a saturated or unsaturated aliphatic hydrocarbon-based radical; or —CO—NRₐ—SO₂— in which the carbonyl group is linked to the nitrogen atom of NX and Rₐ is as defined above;

T represents a hydrogen atom; a halogen atom; a saturated or unsaturated aliphatic hydrocarbon-based group, optionally interrupted with O and/or S and optionally halogenated; nitro; or cyano;

Y may represent any organic substituent when A represents —CO—, and, in the general case, Y is chosen from:

a saturated or unsaturated, aliphatic hydrocarbon-based radical optionally interrupted with O and/or S;

a radical of the formula —(O)ₘ-(alk″)ₙ-Rcy in which m represents 0 or 1; when m represents 0, then n represents 0 and when m represents 1, then n is chosen from 0 and 1; alk″ represents nothing or represents a saturated aliphatic hydrocarbon-based chain; and Rcy represents (i) either a saturated, unsaturated and/or aromatic carbocyclic radical, optionally substituted with one or more substituents chosen from oxo and the radicals R defined below; (ii) or a saturated, unsaturated and/or aromatic heterocyclic radical, optionally substituted with one or more substituents chosen from oxo and the radicals R defined below; it being understood that when A represents CO—NRa, then m represents 0;

a radical

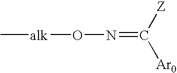

in which alk and Z independently represent a saturated or unsaturated aliphatic hydrocarbon-based chain, Z also possibly representing a hydrogen atom, and Ar₀ represents a saturated, unsaturated and/or aromatic, carbocyclic radical optionally substituted with one or more substituents chosen from oxo and the radicals R defined below, or alternatively Ar₀ represents a saturated, unsaturated and/or aromatic heterocyclic radical optionally substituted with one or more substituents chosen from oxo and the radicals R defined below;

a radical -alk′-W-Cy in which alk′ is as defined above for alk, except that it may also be substituted with one or more radicals G as defined below; W is chosen from O, S, —NH—SO₂—, —NH—CO—, —CO—NH—, —CO— and —SO₂; and Cy represents a saturated or unsaturated aliphatic hydrocarbon-based radical, optionally substituted with one or more radicals G as defined below; or alternatively Cy represents a saturated, unsaturated and/or aromatic carbocyclic radical optionally substituted with one or more substituents chosen from oxo and the radicals R defined below; or alternatively Cy represents a saturated, unsaturated and/or aromatic heterocyclic radical optionally substituted with one or more substituents chosen from oxo and the radicals R defined below; it being understood that when alk′ and Cy do not both represent an unsubstituted saturated or unsaturated aliphatic hydrocarbon-based radical, then W can represent nothing, in which case Cy may represent one of the radicals R defined below; and

a radical -(alk-NH—CO)_{q}-Ar₀ in which alk and Ar₀ are as defined above; and q represents an integer from 1 to 5;

G represents a halogen atom; a cyano group; a nitro group; a hydroxyl group; an amino group; an alkylamino group; a dialkylamino group; an aryl group which is optionally halogenated and/or optionally substituted with alkyl; an alkyl group which is optionally interrupted with O and/or S and optionally halogenated;

R is chosen from a halogen atom; a cyano group; a nitro group; an amino group; an alkylamino group; a dialkylamino group; a dialkylaminoalkoxy group; a dialkylaminoalkylthio group; an aryl group optionally substituted with one or more radicals G; an alkyl group optionally interrupted with O and/or S and optionally halogenated; a hydroxyl group; an alkylthio group substituted with arylsulfonyl in which aryl is optionally substituted with one or more radicals G; an aryloxy group in which aryl is optionally substituted with one or more radicals G; an arylthio group in which aryl is optionally substituted with one or more radicals G; an alkylsulfonyl group; an arylsulfonyl group in which aryl is optionally substituted with one or more radicals G; an alkylcarbonyl group; a heteroaryl group comprising one or more hetero atoms chosen from O, N and S and optionally substituted with one or more radicals G and/or with alkoxycarbonyl; an alkoxycarbonyl group; an alkylcarbonyloxy group; an alkylcarbonylamino group; an alkylenedioxy group; an alkylene group optionally substituted with oxo; an arylalkyl group in which aryl is optionally substituted with one or more radicals G; a cycloalkyl group optionally substituted with one or more radicals G; a cycloalkylalkyl group in which cycloalkyl is optionally substituted with one or more radicals G and/or with arylsulfonylamino in which aryl is itself optionally halogenated;

the stereoisomers thereof, the addition salts thereof with acids or bases and the hydrates and solvates thereof.

Hydrates and solvates are understood as meaning, for example, the hemi-, mono- or dihydrates, solvates are understood as meaning, for example, alcohol addition compounds such as, for example, with methanol or ethanol.

The term “organic substituent (Y)” means any substituent attached to the carbonyl group (A) via a carbon atom, and more particularly a substituent comprising one or more carbon, nitrogen, oxygen, sulfur, phosphorus, halogen, silicon and hydrogen atoms.

In the context of the invention, the term “alkyl” means a linear or branched hydrocarbon-based chain comprising from 1 to 14 carbon atoms, preferably from 1 to 10 and better still from 1 to 6 carbon atoms, for example from 1 to 4 carbon atoms.

Examples of alkyl radicals are methyl, ethyl, propyl, isopropyl, butyl, isobutyl, t-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 2-ethylbutyl, 1-methyl-1-ethylpropyl, heptyl, 1-methylhexyl, 1-propylbutyl, 4,4-dimethylpentyl, octyl, 1-methylheptyl, 2-methylhexyl, 5,5-dimethylhexyl, nonyl, decyl, 1-methylnonyl, 3,7-dimethyloctyl and 7,7-dimethyloctyl.

The term “halogen atom” means chlorine, bromine, iodine or fluorine.

In the context of the invention, the expression “radical substituted with oxo” means the corresponding radical bearing on one of the carbon atoms constituting it a group=O as substituent.

The expression “saturated or unsaturated aliphatic hydrocarbon-based chain” means a linear or branched, preferably C₁—C₁₄ and better still C₁—C₁₀ chain, for example a C₁—C₆ or C₁—C₄ chain.

If this chain is unsaturated, it contains one or more unsaturations, preferably one or two unsaturations. The unsaturations are either of ethylenic or of acetylenic type. They are preferably ethylenic. The unsaturated chains contain at least two carbon atoms.

The alkyl groups are examples of saturated aliphatic hydrocarbon-based chains.

The alkenyl and alkynyl groups are examples of unsaturated aliphatic hydrocarbon-based chains.

The expression “optionally interrupted with O and/or S” means that any carbon atom of the hydrocarbon-based chain may be replaced with an oxygen or sulfur atom, this carbon atom not being able to be located at the free end of the hydrocarbon-based chain. The hydrocarbon-based chain, which may be alkyl, may comprise several oxygen and/or sulfur atoms, the hetero atoms preferably being separated from each other by at least one carbon atom and better still by at least two carbon atoms.

An example of an aliphatic hydrocarbon-based chain interrupted with O or S is alkoxy or thioalkoxy.

The carbocyclic and heterocyclic radicals include mono- and polycyclic radicals; these radicals preferably denote mono-, bi- or tricyclic radicals. In the case of polycyclic radicals, it should be understood that these consist of monocycles fused in pairs (for example ortho-fused or peri-fused), i.e. having at least two carbon atoms in common. Preferably, each monocycle is 3- to 8-membered and better still 5- to 7-membered.

The cycloalkyl groups are an example of saturated carbocyclic radicals and preferably contain from 3 to 18 carbon atoms and better still from 3 to 10 carbon atoms, such as cyclopropyl, cyclobutyl, cyclopentyl, cyclohexyl, cycloheptyl, cyclooctyl, adamantyl or norbomyl radicals.

The unsaturated carbocyclic groups comprise one or more, preferably 1 to 3, ethylenic double bonds, and consist generally of from 6 to 18 and better still from 6 to 10 carbon atoms. Examples of these are cycloalkenyl radicals, and in particular cyclohexenyl.

The aromatic carbocyclic groups are, for example, C₆—C₁₈ aryl groups and in particular phenyl, naphthyl, anthryl and phenanthryl.

It should be understood that the expression “saturated, unsaturated and/or aromatic cyclic radical” means that the same radical may comprise a saturated portion and/or an unsaturated portion and/or an aromatic portion.

Mention will be made, for example, of the case of the following carbocyclic radicals:

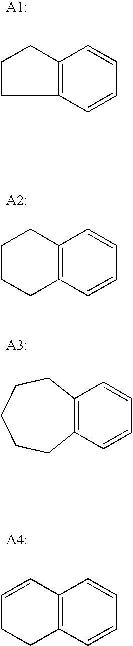

Mention may also be made of the case of the following heterocyclic radicals:

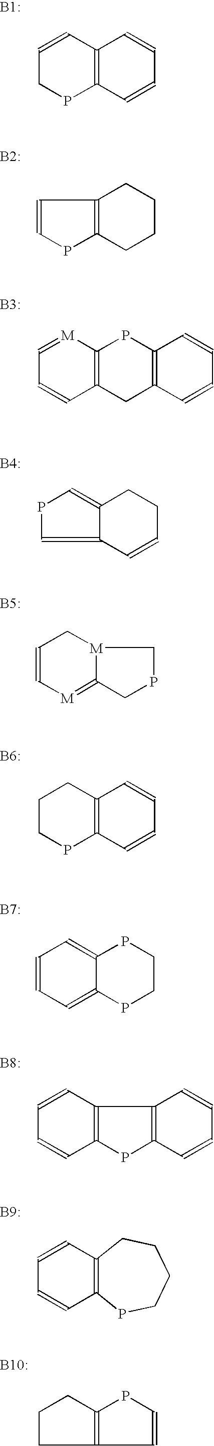

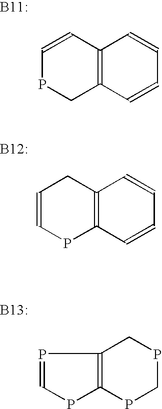

in which P represents O, S or SO₂ and M represents N or C. Preferably, in B1, P represents O; in B2, P represents O or S; in B3, P represents SO₂ or O and M represents C or N; in B4, P represents S; in B5, M represents N and P represents S; in B6, P represents O; in B7, P represents O; in B8, P represents O; in B9, P represents O; in B10, P represents S; in B11, P represents 0; in B12, P represents O; in B13, P represents N.

When M or P represents N, the latter is preferably substituted with a hydrogen atom, an alkyl or an alkylcarbonyl.

The heterocyclic groups comprise hetero atoms generally chosen from O, S and N, optionally in oxidized form (in the case of S and N).

Preferably, each of the monocycles constituting the heterocycle comprises from 1 to 4 hetero atoms, and better still from 1 to 3 hetero atoms.

The following are distinguished in particular:

5- to 7-membered monocyclic heterocycles such as, for example, heteroaryls chosen from pyridine, furan, thiophene, pyrrole, pyrazole, imidazole, thiazole, isoxazole, isothiazole, furazane, pyridazine, pyrimidine, pyrazine, thiazines, oxazole, pyrazole, oxadiazole, triazole and thiadiazole; and also unsaturated and saturated derivatives thereof. Examples of unsaturated 7-membered heterocycles are trithiatriazepines and trithiadiazepines. Examples of saturated 5- to 7-membered heterocycles are in particular tetrahydrofuran, dioxolane, imidazolidine, pyrazolidine, piperidine, dioxane, morpholine, dithiane, thiomorpholine, piperazine, trithiane, oxepine and azepine;

bicyclic heterocycles in which each monocycle is 5- to 7-membered, such as, for example heteroaryls chosen from indolizine, indole, isoindole, benzofuran, benzothiophene, indazole, benzimidazole, benzothiazole, benzofurazane, benzothiofurazane, purine, quinoline, isoquinoline, cinnoline, phthalazine, quinazoline, quinoxaline, naphthyridines, pyrazolotriazine (such as pyrazolo-1,3,4-triazine), pyrazolopyrimidine and pteridine; and also unsaturated and saturated derivatives thereof;

tricyclic heterocycles in which each monocycle is 5- to 7-membered, whether they are completely aromatic, such as, for example, acridine, phenazine carbazole, or not, such as unsaturated and saturated derivatives thereof, phenothiazine or phenoxazine.

It should be understood that the expression “saturated, unsaturated and/or aromatic cyclic radical” means that the same radical may comprise a saturated portion and/or an unsaturated portion and/or an aromatic portion.

Preferred meanings of X, T, A, G and R are in particular the following:

X represents a hydrogen atom; (C₁—C₁₄)alkyl, preferably (C₁—C₁₀)alkyl; or a group -A-Y.

A represents —CO—; —SO₂-; —CO—NRₐ- in which the carbonyl group is linked to the nitrogen atom of NX and Rₐ represents a hydrogen atom or (C₁—C₁₄)alkyl, preferably (C₁—C₁₀)alkyl; or —CO—NRₐ—SO₂- in which the carbonyl group is linked to the nitrogen atom of —NX and Rₐ is as defined above.

T represents H; a halogen atom (and preferably chlorine or fluorine); a cyano group; a nitro group; an optionally halogenated (C₁—C₁₄)alkoxy, preferably an optionally halogenated (C₁—C₁₀)alkoxy (and preferably trifluoromethoxy); an optionally halogenated (C₁—C₁₄)thioalkoxy group, preferably (C₁—C₁₀)thioalkoxy; an optionally halogenated (C₁—C₁₄)alkyl, preferably an optionally halogenated (C₁—C₁₀)alkyl (and in particular methyl).

T represents H; an optionally halogenated (C₁—C₁₄)alkoxy group; or an optionally halogenated (C₁—C₁₄)thioalkoxy group.

G represents halogen; hydroxyl; optionally halogenated (C₁—C₁₄)alkoxy, preferably optionally halogenated (C₁—C₁₀)alkoxy; optionally halogenated (C₁—C₁₄)alkyl, preferably optionally halogenated (C₁—C₁₀)alkyl; nitro; cyano; amino; (C₁—C₁₄)alkylamino, preferably (C₁—C₁₀)alkylamino; di(C₁—C₁₄)alkylamino, preferably di(C₁—C₁₀)alkylamino; (C₆—C₁₀)aryl which is optionally halogenated and/or optionally substituted with (C₁—C₁₄)alkyl;

R is chosen from a halogen atom; cyano; hydroxyl; nitro; optionally halogenated (C₁—C₁₀)alkyl; optionally halogenated (C₁—C₁₀)alkoxy; (C₁—C₁₀)alkylthio optionally substituted with (C₆—C₁₀)arylsulfonyl in which aryl is optionally substituted with one or more radicals G; (C₆—C₁₀)aryloxy in which aryl is optionally substituted with one or more radicals G; (C₆—C₁₀)arylthio in which aryl is optionally substituted with one or more radicals G; (C₁—C₁₀)alkylsulfonyl; (C₆—C₁₀)arylsulfonyl in which aryl is optionally substituted with one or more radicals G; 5- to 7-membered heteroaryl comprising one or more hetero atoms chosen from O, N and S and optionally substituted with one or more radicals G and/or with (C₁—C₁₀)alkoxycarbonyl; (C₁—C₁₀)alkoxycarbonyl; (C₁—C₁₀)alkylcarbonylamino; di(C₁—C₁₀)alkylamino; (C₂—C₄)alkylenedioxy; (C₃—C₅)alkylene optionally substituted with oxo; (C₆—C₁₀)aryl-(C₁—C₁₀)alkyl in which aryl is optionally substituted with one or more radicals G; amino; (C₁—C₁₀)alkylamino; di(C₁—C₁₀)alkylamino; optionally halogenated (C₆—C₁₀)aryl; (C₁—C₁₀)alkylcarbonyl, preferably (C₁—C₆)alkylcarbonyl; (C₃—C₈)cycloalkyl-(C₁—C₆)alkyl in which cycloalkyl is itself substituted with (C₆—C₁₀)arylsulfonylamino in which aryl is optionally halogenated.

The term “alkylene” means a linear or branched divalent hydrocarbon-based radical comprising 1 to 6 carbon atoms, preferably 1 to 4 carbon atoms and better still 1 or 2 carbon atoms, originating from the removal of two hydrogen atoms on two different carbon atoms of a saturated carbide. The groups —CH₂— and CH₂—CH₂— constitute alkylene radicals that are particularly preferred.

The term “alkenylene” means a linear or branched divalent hydrocarbon-based radical comprising 2 to 6 carbon atoms, preferably 2 to 4 carbon atoms and better still 2 or 3 carbon atoms, originating from the removal of two hydrogen atoms on two different carbon atoms of an unsaturated carbide comprising one or more double bonds.

Examples of alkenylenes are —CH=CH— and CH=CH—CH₂-.

The terms “optionally halogenated alkyl” and “optionally halogenated alkoxy” mean alkyl or alkoxy, respectively, substituted with one or more halogen atoms.

A preferred haloalkyl group is the trifluoromethyl group, and a preferred haloalkoxy group is trifluoromethoxy.

Advantageously, the compounds that are preferred are those of the formula (I) in which one, two, three, four or five of the substituents X, T, A, G and R take(s) one of the preferred meanings given above.

As regards Y, preferred meanings are in particular the following:

a) (C₁—C₁₀)alkyl;

b) (C₁—C₁₀)alkoxy-(C₁—C₁₀)alkyl;

c) (C₁—C₁₀)alkoxy-(C₁—C₁₀)alkoxy;

d) coumarinyl optionally substituted with one or more radicals G as defined above;

e) a group

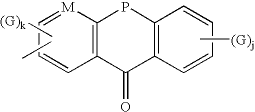

in which j and k independently represent an integer from 0 to 4;

M represents N or C;

P represents SO₂ or O;

G is as defined above;

f) a group

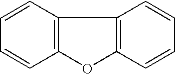

optionally substituted with one or more radicals G as defined above;

g) a group

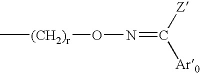

in which r is an integer from 0 to 6;

Z′ represents a hydrogen atom or (C₁—C₁₀)alkyl;

Ar′ₒ represents (C₆—C₁₀)aryl optionally substituted with one or more radicals G as defined above;

h) a group -alks-W′-Cy′ in which:

alks represents (C₁—C₁₀)alkylene optionally substituted with (C₆—C₁₀)aryl, itself optionally substituted with one or more radicals G as defined above;

W′ represents O, S, —NH—SO₂—, —NH—CO—, —CO—NH—, —CO— or —SO₂—;

Cy′ represents (C₁—C₁₄)alkyl optionally substituted with (C₆—C₁₀)aryl and/or amino; (C₆—C₁₀)aryl optionally substituted with one or more radicals G as defined above; 5- to 7-membered heteroaryl comprising one or more hetero atoms chosen from O, N and S, optionally substituted with one or more radicals G as defined above; or a saturated 5- to 7-membered heterocycle comprising one or more hetero atoms chosen from O, N and S, optionally substituted with one or more radicals G as defined above and/or with an oxo group;

i) a group -(alks′-NH—CO)_{q}-(C₆—C₁₀)aryl in which alks′ represents (C₁—C₆)alkylene; q represents an integer from 1 to 5; and aryl is optionally substituted with one or more radicals G;

j) (C₂—C₁₀)alkenyl optionally substituted with a group —NH—CO-(C₁—C₁₀)alkyl; with a group (C₆—C₁₀)aryl itself optionally substituted with one or more radicals G as defined above; with a 5- to 7-membered heteroaryl group comprising one or more hetero atoms chosen from O, N and S, itself optionally substituted with one or more radicals G as defined above; and/or with a group —CO—NH—(C₁—C₁₀)alkyl;

k) -(alk″)ₚ-Ar′ in which

•p represents the integer 0 or 1;

alk″ represents (C₁—C₆)alkylene or (C₂—C₆)alkenylene;

•Ar′ represents (C₃—C₈)cycloalkyl optionally substituted with one or more radicals G as defined above and/or with oxo, and optionally fused to (C₆—C₁₀)aryl, the said aryl nucleus optionally being substituted with one or more radicals G;

• or alternatively Ar′ represents heteroaryl with a monocyclic, bicyclic or tricyclic nucleus comprising one or more hetero atoms chosen from O, N and S, the said hetero atoms N and S optionally being in oxidized form, in which each ring of the said monocyclic, bicyclic or tricyclic nucleus is 5- to 7-membered, the rings not directly linked to the group —NX—A-(alk″)ₚ- optionally being partially or totally hydrogenated, the said heteroaryl optionally being substituted with one or more radicals R as defined above and/or where appropriate with an oxo group, it being understood that heteroaryl also denotes the mesomeric performs of the mono-, bi- and tricyclic nuclei defined above;

• or alternatively Ar′ represents 5- to 7-membered saturated or unsaturated heterocycle comprising one or more hetero atoms chosen from N, O and S and optionally substituted with one or more oxo radicals and/or radicals G as defined above, the nitrogen atom also possibly being optionally substituted with (C₁—C₆)alkylcarbonyl; with (C₆—C₁₀)arylsulfonyl; or with (C₆—C₁₀)aryl-(C₁—C₆)alkyl, in which the aryl portions are optionally substituted with one or more radicals G as defined above; the said heterocycle optionally being fused to a (C₆—C₁₀)aryl nucleus optionally substituted with one or more radicals G as defined above;

• or alternatively Ar′ represents (C₆—C₁₀)aryl optionally substituted with one or more radicals R as defined below, or, when p is other than 0, aryl is optionally substituted with (C₃—C₈)cycloalkyl-(C₁—C₆)alkyl in which cycloalkyl is itself substituted with (C₆—C₁₀)arylsulfonylamino in which aryl is optionally halogenated.

Examples of mesomeric forms of heteroaryl with a monocyclic, bicyclic or tricyclic nucleus are, in particular:

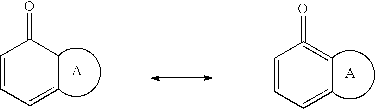

in which A represents a 5- to 7-membered ring and in which each sp² carbon atom may be replaced with N, and each Sp³ carbon atom may be replaced with O, S or NH, or alternatively a substituted nitrogen atom.

When Y takes one of these preferred meanings, it is particularly desirable for A to represent CO.

Similarly, it is preferable for G and R to be as defined below:

G represents halogen; optionally halogenated (C₁—C₆)alkyl; optionally halogenated (C₁—C₆)alkoxy; nitro; or cyano;

R is chosen from a halogen atom; cyano; hydroxyl; nitro; optionally halogenated (C₁—C₁₀)alkyl; optionally halogenated (C₁—C₁₀) alkoxy; (C₁—C₁₀)alkylthio optionally substituted with (C₆—C₁₀)arylsulfonyl in which aryl is optionally substituted with one or more radicals G; (C₆—C₁₀)aryloxy in which aryl is optionally substituted with one or more radicals G; (C₆—C₁₀)arylthio in which aryl is optionally substituted with one or more radicals G; (C₁—C₁₀)alkylsulfonyl; (C₆—C₁₀)arylsulfonyl in which aryl is optionally substituted with one or more radicals G; 5- to 7-membered heteroaryl comprising one or more hetero atoms chosen from O, N and S and optionally substituted with one or more radicals G and/or with (C₁—C₁₀)alkoxycarbonyl; (C₁—C₁₀)alkoxycarbonyl; (C₁—C₁₀)alkylcarbonylamino; di(C₁—C₁₀)alkylamino; (C₂—C₄)alkylenedioxy; (C₃—C₅)alkylene optionally substituted with oxo; and (C₆—C₁₀)aryl-(C₁—C₁₀)alkyl in which aryl is optionally substituted with one or more radicals G.

Preferred examples of radicals Y=h) as defined above are, more specifically:

h-1) a group -alks-He—Cy′ in which alks represents (C₁—C₆)alkylene, He represents O or S. and Cy′ represents (C₆—C₁₀)aryl optionally substituted with one or more radicals G; heteroaryl optionally substituted with one or more radicals G; or (C₁—C₁₄)alkyl.

For example, the radical -alks-He-Cy′ represents

a group -alks-O—Cy′ in which alks represents (C₁—C₆)alkylene, and Cy′ represents phenyl optionally substituted with cyano, nitro or (C₁—C₆)alkoxy;

a group alks-S-Cy′ in which alks represents (C₁—C₆)alkylene and Cy′ represents phenyl optionally substituted with cyano; or pyridyl optionally substituted with nitro or (C₁—C₆)alkoxy; or alternatively (C₁—C₁₄)alkyl;

h-2) a group -alks-NH—SO₂-Cy′ in which alks represents (C₁—C₁₀)alkylene; Cy′ represents heteroaryl optionally substituted with one or more radicals G.

For example, -alks-NH—SO₂-Cy′ is such that alks represents alkylene; Cy′ represents thienyl optionally substituted with halogen;

h-3) a group -alks-NH—CO—Cy′ in which alks represents (C₁—C₆)alkylene; Cy′ represents (C₁—C₁₄)alkyl; (C₆—C₁₀)aryl optionally substituted with one or more radicals G; saturated heteroaryl optionally substituted with one or more radicals G; saturated heterocycle optionally substituted with one or more radicals G and/or oxo; or (C₁—C₆)alkyl optionally substituted with amino and/or (C₆—C₁₀)aryl.

Preferably, in this group, alks represents (C₁—C₆)alkylene optionally substituted with amino and/or phenyl; and Cy′ represents phenyl optionally substituted with halogen; (C₁—C₆)alkyl; furyl; 2-oxopyrrolidinyl;

h-4) a group -alks-CO—NH-Cy′ in which alks represents (C₁—C₆)alkylene; Cy′ represents phenyl optionally substituted with one or more radicals G. In particular, alks represents (C₁—C₆)alkylene and Cy′ represents phenyl;

h-5) a group -alks-CO-Cy′ in which alks represents (C₁—C₁₀)alkylene; Cy′ represents heteroaryl optionally substituted with one or more radicals G.

Preferably, alks represents (C₁—C₆)alkylene and Cy′ represents phenyl optionally substituted with (C₁—C₆)alkyl;

h-6) a group -alks-SO₂-Cy′ in which alks represents (C₁—C₆)alkylene; Cy′ represents (C₆—C₁₀)aryl optionally substituted with one or more radicals G.

Preferably, alks represents (C₁—C₆)alkylene and Cy′ represents phenyl optionally substituted with halogen.

When Y=i) as defined above, it is preferred for i) to represent:

i-1) a group -(alks′-NH—CO)ₚ—(C₆—C₁₀)aryl in which alks′ represents (C₁—C₆)alkylene; and p represents 2 or 3.

Preferably, alkylene is C₁—C₃ and aryl represents phenyl optionally substituted with nitro.

When Y=j) as defined above, it is preferred for alkyl to be C₁—C₆; for aryl to represent phenyl; for heteroaryl to represent thienyl; for alkenyl to be C₂—C₆; for phenyl to the optionally substituted with nitro, halogen, (C₁—C₆)alkyl or (C₁—C₆)alkoxy; for thienyl to be unsubstituted.

When Y=k) as defined above and p is other than 0, then it is preferred for alk″ to comprise from 1 to 3 carbon atoms; and for Ar′ to represent:

(C₃—C₈)cycloalkyl optionally substituted with oxo and optionally fused to a phenyl nucleus, itself optionally substituted with one or more radicals G; and example of this is the radical A₁ defined above optionally substituted with oxo and/or one or more radicals G;

5- to 7-membered heteroaryl, such as thienyl optionally substituted with one or more radicals G;

phenyl optionally substituted with one or more radicals G and/or with (C₃—C₈)cycloalkyl-(C₁—C₆)alkyl in which cycloalkyl is itself substituted with (C₆—C₁₀)arylsulfonylamino in which aryl is optionally halogenated. Preferably, in this case, aryl represents phenyl;

a 5- to 7-membered heterocyclic radical comprising one or two hetero atoms chosen from O, N and S and fused to a phenyl nucleus, the said radical optionally being substituted with one or more radicals G. Preferably, the hetero atoms is O. Preferably, the said radical has the formula B9.

When Y=k) as defined above and p is 0, then Ar′ advantageously takes one of the following meanings:

(C₃—C₈)cycloalkyl optionally fused to phenyl and optionally substituted with one or more oxo radicals and/or radicals G, the phenyl nucleus itself optionally being substituted with one or more radicals G;

phenyl optionally substituted with one or more radicals R (R preferably being chosen from aikoxy; halogen; nitro; alkoxycarbonyl; alkylcarbonylamino; hydroxyl; optionally halogenated alkyl; alkylsulfonyl; 5- to 7-membered heteroaryl optionally substituted with one or more radicals G, such as, for example, optionally substituted pyrazolyl; alkylenedioxy);

5- to 7-membered heteroaryl optionally substituted with one or more radicals R (R preferably being chosen from thioalkoxy optionally substituted with phenylsulfonyl in which phenyl is itself substituted with one or more radicals G; phenlyoxy optionally substituted with one or more radicals G; optionally halogenated alkyl; halogen; alkylsulfonyl; NO₂; optionally halogenated alkoxy; 5- to 7-membered heteroaryl optionally substituted with alkoxycarbonyl and/or with one or more radicals G; phenylthio optionally substituted with one or more radicals G). Preferably, heteroaryl represents pyrimidine, pyrazole, pyridine, oxazole, thiadiazole, thienyl, pyrrole, furyl, thiazole, triazole or imidazole;

5- to 7-membered saturated and/or unsaturated heterocycle optionally fused to a phenyl nucleus, the whole optionally being substituted with one or more radicals R and/or oxo, R preferably being chosen from alkylcarbonyl; phenylalkyl; phenylsulfonyl in which phenyl is optionally substituted with one or more radicals G; alkoxy.

When this radical is heteroaryl, it is preferably pyrrolyl or piperidyl. When this radical is bicyclic, it preferably has the formula B1 or B6 in which P represents O.

bicyclic heteroaryl in which each monocycle is 5- to 7-membered, the monocycle not directly linked to —NX—A— optionally being partially hydrogenated, the said radical optionally being substituted with one or more radicals R and/or oxo, R preferably being chosen from nitro, alkyl, alkylsulfonyl and alkoxy.

This radical preferably has the formula (it being understood that it may be substituted): benzofuryl, (tetrahydrobenzo)furyl, (dihydrobenzo)thienyl, pyrazolotriazine, thiazolidinopyrimidine, pyrazolopyrimidine.

When the compounds of the formula I are such that A represents SO₂; —CO—NRₐ—; or —CO—NRₐ—SO₂—; then preferred meanings of Y are as follows:

Y represents (C₁—C₁₀)alkyl optionally substituted with (C₁—C₁₀)alkylsulfonyl; (C₃—C₈)cycloalkyl; or alternatively -(alk″)_{q}-Ar″

in which

q is the integer 0 or 1,

alk″ represents (C₁—C₆)alkylene or (C₂—C₆)alkenylene, and

Ar″ represents (C₆—C₁₀)aryl optionally substituted with one or more radicals R as defined above;

or alternatively Ar″ represents heteroaryl with a monocyclic, bicyclic or tricyclic nucleus comprising one or more hetero atoms chosen from O, N and S, the hetero atoms N and S optionally being in oxidized form, each ring of the said monocyclic, bicyclic or tricyclic nucleus being 5- to 7-membered, and the said heteroaryl optionally being substituted with one or more radicals R as defined above.

In this preferred definition of Y, heteroaryl is preferably monocyclic (and, for example, pyridyl, thienyl, imidazolyl, pyrazolyl or thiazolyl) or bicyclic (and, for example, benzothienyl, quinolyl, benzoxadiazolyl or benzothiadiazolyl).

When Y takes one of these preferred meanings:

R is advantageously chosen from:

halogen; optionally substituted (C₁—C₁₀)alkyl; optionally halogenated (C₁—C₁₀)alkoxy; nitro; (C₁—C₁₀)alkoxycarbonyl; (C₁—C₁₀)alkylcarbonyl; (C₁—C₁₀)alkylcarbonylamino; di(C₁—C₁₀)alkylamino; cyano; (C₁—C₁₀)alkylthio; (C₆—C₁₀)aryloxy in which aryl is optionally substituted with one or more radicals G as defined above; (C₁—C₁₀)alkylsulfonyl; (C₆—C₁₀)arylsulfonyl optionally substituted with one or more radicals G as defined above; and 5- to 7-membered heteroaryl comprising one or more hetero atoms chosen from O, N and S and optionally substituted with one or more radicals G as defined above and in which the nitrogen and sulfur atoms are optionally in oxidized form;

G is advantageously chosen from:

halogen, (C₁—C₆)alkyl, (C₁—C₆)alkoxy, nitro and cyano;

and T is advantageously chosen from:

a hydrogen atom, (C₁—C₁₀)alkoxy, (C₁—C₁₀)alkylthio or (C₁—C₁₀)alkyl which is optionally halogenated.

A preferred subgroup of compounds of the formula I in which A represents SO₂ is such that:

Y represents:

(C₁—C₆)alkyl;

phenyl optionally substituted with one or more halogen, nitro, cyano, optionally halogenated (C₁—C₆)alkyl, optionally halogenated (C₁—C₆)alkoxy, (C₁—C₆)alkylcarbonylamino, (C₁—C₆)alkylcarbonyl, (C₁—C₆)alkoxycarbonyl, di(C₁—C₆)alkylamino, (C₁—C₆)alkylsulfonyl, or phenoxy optionally substituted with one or more radicals G as defined above;

naphthyl optionally substituted with one or more di(C₁—C₆)alkylamino;

phenyl-(C₁—C₆)alkyl in which phenyl is optionally substituted with one or more radicals G as defined above;

(C₁—C₆)alkyl optionally substituted with (C₁—C₆)alkylsulfonyl;

(C₃—C₈)cycloalkyl;

phenyl-(C₂—C₆)alkenyl in which phenyl is optionally substituted with one or more radicals G as defined above;

5- to 7-membered monocyclic heteroaryl chosen from imidazolyl, pyrazolyl, thiazolyl, thienyl, pyridyl, pyrazolyl, furyl, N-oxypyridyl, pyrazinyl, pyrimidinyl and isoxazolyl, the said heteroaryl optionally being substituted with one or more radicals chosen from (C₁—C₆)alkoxy, (C₁—C₆)alkylthio, halogen, (C₁—C₆)alkyl, di(C₁—C₆)alkylamino, (C₁—C₆)alkylcarbonylamino, (C₁—C₆)alkoxycarbonyl, phenylsulfonyl and pyridyl;

bicyclic heteroaryl chosen from quinolyl, isoquinolyl, benzothienyl and a radical of the formula:

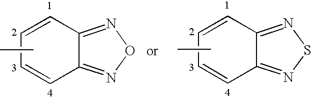

the said bicyclic heteroaryl optionally being substituted with one or more radicals G as defined above;

or heteroaryl-(C₁—C₆)alkyl in which heteroaryl represents 5- to 7-membered monocyclic heteroaryl as defined above, the said heteroaryl optionally being substituted with one or more radicals G as defined above.

More particularly, when A represents SO₂, Y represents quinolyl optionally substituted with one or more radicals G; optionally substituted pyridyl; optionally substituted pyrimidinyl.

Another preferred subgroup of compounds of the formula I consists of compounds in which:

X represents H;

A represents SO₂;

Y represents

phenyl optionally substituted with one or more radicals chosen from nitro, halogen, optionally halogenated (C₁—C₆)alkyl and optionally halogenated (C₁—C₆)alkoxy;

pyridyl optionally substituted with one or more radicals chosen from (C₁—C₆)alkoxy, halogen and (C₁—C₆)alkyl;

T represents a hydrogen atom or (C₁—C₆)alkoxy.

When, among the compounds of the formula I, A represents —CO—NRₐ— or —CO—NRₐ—SO₂—, then it is preferred for Y to represent phenyl optionally substituted with one or more halogen, nitro, cyano, optionally halogenated (C₁—C₆)alkyl, optionally halogenated (C₁—C₆)alkoxy, (C₁—C₆)alkylcarbonylamino, (C₁—C₆)alkylcarbonyl, (C₁—C₆)alkoxycarbonyl, di(C₁—C₆)alkylamino or phenoxy optionally substituted with one or more radicals G as defined above.

For all the compounds of the formula I, it is preferred for X to represent H or (C₁—C₁₀)alkyl.

Nevertheless, when X represents -A-Y, Y then preferably takes one of the following meanings:

Y represents

phenyl optionally substituted with one or more halogen, nitro, cyano, optionally halogenated (C₁—C₆)alkyl, optionally halogenated (C₁—C₆)alkoxy, (C₁—C₈)alkylcarbonylamino, (C₁—C₆)alkylcarbonyl, (C₁—C₆)alkoxycarbonyl, di(C₁—C₆)alkylamino, (C₁—C₆)alkylsulfonyl or phenoxy optionally substituted with one or more radicals G as defined above;

5- to 7-membered monocyclic heteroaryl chosen from imidazolyl, pyrazolyl, thiazolyl, thienyl, pyridyl, pyrazolyl, furyl, N-oxypyridyl, pyrazinyl, pyrimidinyl and isoxazolyl, the said heteroaryl optionally being substituted with one or more radicals chosen from (C₁—C₆)alkoxy, (C₁—C₆)alkylthio, halogen, (C₁—C₆)alkyl, di(C₁—C₆)alkylamino, (C₁—C₆)alkylcarbonylamino, (C₁—C₆)alkoxycarbonyl, phenylsulfonyl and pyridyl;

bicyclic heteroaryl chosen from quinolyl, benzothienyl and a radical chosen from

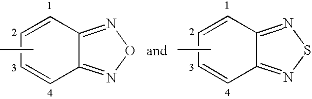

the said bicyclic heteroaryl optionally being substituted with one or more radicals G as defined above.

Another preferred subgroup of compounds of the formula I consists of compounds in which:

A represents CO;

Y represents pyridyl; phenyl optionally substituted with one or more radicals G; or alternatively alkylphenyl optionally substituted with one or more radicals G.

A subgroup of compounds of the formula I that are particularly active consists of compounds of the formula:

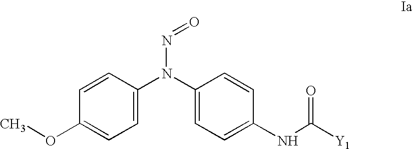

in which:

Y₁ is chosen from pyridyl optionally substituted with one or more substituents chosen from oxo and the radicals R defined above; pyrimidinyl optionally substituted with one or more substituents chosen from oxo and the radicals R defined above; and benzyl optionally substituted with one or more substituents chosen from oxo and the radicals R defined above.

Another subgroup of compounds of the formula I that are particularly active consists of compounds of the formula:

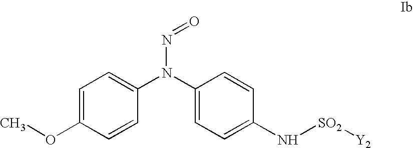

in which:

Y₂ represents 3-pyridyl optionally substituted with one or more substituents chosen from oxo and the radicals R defined above; phenyl optionally substituted with one or more substituents chosen from oxo and the radicals R defined above; >CH=CH—Cy^{o} in which Cy^{o} represents phenyl optionally substituted with one or more radicals G.

Another preferred subgroup of compounds of the formula I consists of compounds in which:

A=CO and Y=—(O)ₘ-(alk″)ₙ-Rcy (carbamates).

Rcy is preferably phenyl substituted with fluorine, chlorine, methoxy, cyano, pyridine, cinnamyl, 2-methyl-1-propene, nitrate or oxazolyl optionally substituted with methyl.

The invention is directed not only towards the compounds of the formula I, but also towards the salts thereof.

When the compound of the formula I comprises an acid function, and, for example, a carboxylic function, this function may form a salt with a mineral or organic base.

As examples of salts with organic or mineral bases, mention may be made of the salts formed with metals and in particular alkali metals, alkaline-earth metals and transition metals (such as sodium, potassium, calcium, magnesium or aluminium) or with bases such as ammonia or secondary or tertiary amines (such as diethylamine, triethylamine, piperidine, piperazine or morpholine) or with basic amino acids, or with osamines (such as meglumine) or with amino alcohols (such as 3-aminobutanol and 2-aminoethanol.

When the compound of the formula I comprises a basic function, and for example a nitrogen atom, this compound may form a salt with an organic or mineral acid.

The salts with organic or mineral acids are, for example, the hydrochloride, hydrobromide, sulfate, hydrogen sulfate, dihydrogen phosphate, citrate, maleate, fumarate, 2-naphthalenesulfonate and para-toluenesulfonate.

The invention also covers salts which allow a suitable separation or crystallization of the compounds of the formula I, such as picric acid, oxalic acid or an optically active acid, for example tartaric acid, dibenzoyltartaric acid, mandelic acid or camphorsulfonic acid. However, a preferred subgroup of salts consists of salts of the compounds of the formula I with pharmaceutically acceptable acids or bases.

Formula I includes all the types of geometrical isomers and stereoisomers of the compounds of the formula I.

The compounds illustrated in the examples are preferred. Among these, mention will be made more specifically of the compounds of Examples 1, 4, 6, 85, 86, 100, 243 and 251.

Among these, particularly active compounds which may be mentioned are:

N-nitroso[phenyl][4-(3-pyridylsulfonylamino)phenyl]amine;

N-nitroso[phenyl][4-(phenylsulfonylamino)phenyl]amine;

N-nitroso[4-methoxyphenyl][4-(3-pyridylcarbonylamino)phenyl]amine;

N-nitroso[4-methoxyphenyl][4-{(4-methoxyphenyl)sulfonylamino}phenyl]-amine; and

N-nitroso[4-methoxyphenyl][4-{(3-nitrophenyl)carbonylamino}phenyl]-amine.

Mention may also be made of the compounds of the formula I in which:

a) T=4-OCH₃; X=—CH₃; A=CO; and Y=3-pyridyl;

b) T=H; X=A-Y; A=CO; and Y=2-thienyl;

c) T=H; X=A-Y; A=SO₂; and Y=phenyl;

d) T=H; X=H; Y=CO—NH—SO₂; and Y=phenyl.

The compounds of the invention may be simply prepared by nitrosation of the corresponding compounds of the formula II of the formula:

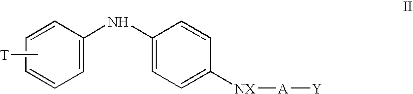

in which T, X, A and Y are as defined above for formula 1, by the action of a suitable nitrosating agent.

Particularly advantageous examples of nitrosating agents are an alkali metal nitrite (and in particular sodium or potassium nitrite) or a C₁—C₄ alkyl nitrite.

A preferred alkali metal nitrite which may be mentioned is sodium nitrite.

A preferred alkyl nitrite which may be mentioned is ethyl nitrite.

Nevertheless, a person skilled in the art can use any nitrosating agent known in the art, such as AgONO, BF₄NO, HOSO₃NO, nBuONO and tBuONO.

The amount of nitrosating agent required depends on the nature of the nitrosating agent used and on the reactivity of the substrate of the formula II. It is at least stoichiometric. In general, the molar ratio of the nitrosating agent to the substrate of the formula II ranges between 1 and 30 equivalents and preferably between 1 and 20 equivalents.

When the nitrosating agent is and alkali metal nitrite, a person skilled in the art may readily adapt the reaction conditions so as to use only 1 to 10, preferably from 1 to 5 and better still from 1 to 3 equivalents of nitrite relative to the substrate of the formula II.

When the nitrosating agent is an alkyl nitrite, it is preferable to perform the process in the presence of 10 to 25 molar equivalents of nitrite, and preferably from 15 to 20 molar equivalents, relative to the amount of substrate of the formula II.

The choice of solvent and the temperature conditions depend in particular on the type of nitrosating agent selected for the reaction.

When the nitrosating agent is AgONO, nBuONO or tBuONO, the solvent is advantageously chosen from a cyclic or non-cyclic ether (such as diethyl ether, diisopropyl ether, tetrahydrofuran, dioxane, dimethoxyethane or diethylene glycol dimethyl ether), an aliphatic or aromatic halogenated hydrocarbon (such as chloroform, carbon tetrachloride, dichloroethane, chlorobenzene or dichlorobenzene). Preferably, the solvent is tetrahydrofuran, diethyl ether or chloroform.

The reaction temperature will generally the maintained between 15 and 70° C. and better still between 17 and 60° C., in the case of AgONO, nBuONO and tBuONO.

More particularly, in the case of AgONO and nBuONO, the process will be performed in tetrahydrofuran or diethyl ether at a temperature of between 15 and 30° C., for example between 18 and 25° C.

In the case of tBuONO, the process will preferably be performed in chloroform at a temperature of between 40 and 65° C., for example between 50 and 60° C.

When the nitrosating agent is AgONO, it is desirable to add thionyl chloride to the reaction medium.

When the nitrosating agent is HOSO₃NO, the reaction is preferably carried out in an alkali metal salt of a lower (C₁—C₅) carboxylic acid, such as sodium acetate, at a reaction temperature of between −10° C. and 30° C. and better still between −5° C. and 25° C.

When the nitrosating agent is BF₄NO, a suitable solvent is a nitrile such as acetonitrile or isobutyronitrile. It is desirable to add pyridine or N-dimethylaminopyridine to the reaction medium, the reaction temperature being maintained between −30° C. and 10° C. and preferably between −25° C. and 5° C.

When the nitrosating agent is an alkali metal nitrite, the nitrosation reaction is preferably carried out in a strongly polar protic medium. Advantageously, the reaction medium contains water and a Bronsted or Lewis acid.

Suitable acids are a hydrohalic acid (such as HCl), sulfuric acid, Al₂(SO₄)₃ and acetic acid, and mixtures thereof.

According to one particular embodiment of the invention, an aliphatic alcohol of (C₁—C₄)alkanol type (such as methanol or butanol) may be added.

Thus, a suitable reaction medium which may be selected is one of the following systems:

a mixture of methanol, water, hydrochloric acid and sulfuric acid;

a mixture of water and sulfuric acid;

a mixture of water and acetic acid;

a mixture of water, butanol and hydrochloric acid;

a mixture of water and Al₂(SO₄)₃, or

a mixture of water and hydrochloric acid.

Advantageously, the reaction of the alkali metal nitrite with the substrate of the formula II is carried out in a mixture of acetic acid and water, the ratio of the acetic acid to water ranging between 80:20 and 20:80 and preferably between 60:40 and 40:60, for example a 50:50 mixture. According to one preferred embodiment, the alkali metal nitrite, predissolved in water, is added dropwise to a solution of the substrate of the formula 11 in acetic acid.

The reaction of the alkali metal nitrite with the substrate of the formula II is carried out at a temperature which depends on the reactivity of the species present; this temperature generally ranges between −10° C. and 50° C. and preferably between −5° C. and 25° C.

When the nitrosation reaction is carried out in a mixture of acetic acid and water, a temperature of between 15° C. and 25° C. is particularly suitable.

The reaction of the alkyl nitrite with the substrate of the formula 11 is preferably carried out in the presence of a C₁—C₄ alkanol in a polar aprotic solvent.

Suitable alkanols which may be mentioned are methanol, ethanol, isopropanol and tert-butanol, ethanol being particularly preferred.

Polar solvents that are preferred are halogenated hydrocarbons, such as methylene chloride, chloroform, carbon tetrachloride, dichloroethane, chlorobenzene or dichlorobenzene; ethers such as diethyl ether, diisopropyl ether, tetrahydrofuran, dioxane, dimethoxyethane or diethylene glycol dimethyl ether; nitrites such as acetonitrile or isobutyronitrile; amides such as formamide, dimethylformamide, dimethylacetamide, N-methyl-2-pyrrolidinone or hexamethylphosphoramide; and mixtures of these solvents in any proportions.

Advantageously, the nitrosation reaction (when an alkyl nitrite is used as nitrosating agent) is carried out in a mixture based on an aliphatic halogenated hydrocarbon and a nitrile, and, for example, in a 90:10 to 50:50 and preferably a 90:10 to 70:30 mixture of chloroform and acetonitrile, in the presence of ethanol.

The amount of alkanol which needs to be incorporated into the reaction medium is not critical according to the invention. It generally represents 5% to 50% by weight of the reaction medium, and preferably from 5% to 25% by weight.

When the nitrosating agent is an alkyl nitrite, the reaction temperature is generally maintained between −20° C. and 20° C. and preferably between −10° C. and 10° C., for example between 0° C. and 5° C.

According to one preferred embodiment of the invention, a solution of the alkyl nitrate in the alkanol is added dropwise to the substrate of the formula II predissolved in the selected polar solvent.

As a variant, the reaction is carried out in a strongly polar medium consisting of a mixture of a C₁—C₄ aliphatic carboxylic acid ((C₁—C₄)alkyl—COOH), the corresponding acid anhydride and the corresponding alkali metal carboxylate salt, in the presence of P₂O₅. By way of example, a reaction medium consisting of acetic acid, acetic anhydride, potassium acetate and P₂O₅ may be selected. In this case, the reaction temperature is advantageously maintained between 10° C. and 100° C. and preferably between 15° C. and 85° C.

The compounds of the formula II may be prepared by carrying out one of the following processes.

A—Preparation of the Compounds of the Formula II in Which A Represents CO and X is H or Alkyl.

Variant A1:

Scheme 1 illustrates one route which is particularly preferred for synthesizing the compounds of the formula II.

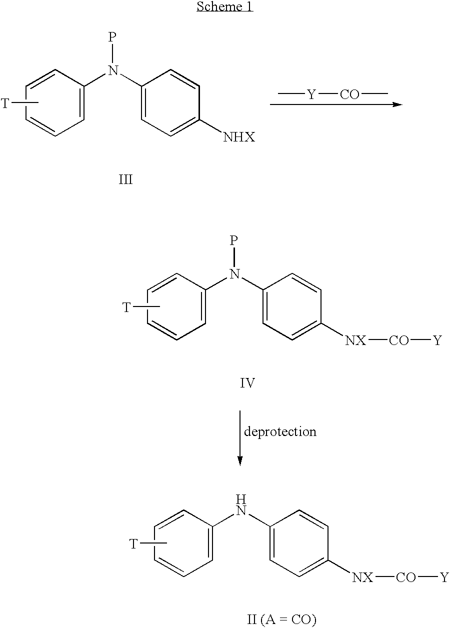

In this scheme, T and Y are as defined the formula II, X represents H or (C₁—C₁₀)alkyl, P represents a protecting group for the amine function and Z represents OH or a residue of a carboxylic acid-activating group as defined below.

Groups that are suitable for protecting the amino function are acyl groups of the type R—CO (in which R is a hydrogen atom or an alkyl, cycloalkyl, aryl, arylalkyl or heteroarylalkyl radical, R optionally being substituted with alkyl, alkoxy or halogen), the groups forming urea of the formula —CO—NA₂B₂ or the groups forming urethane of the formula —CO—OA₂ (in which A₂ and B₂ are, independently, alkyl, aryl, arylalkyl or cycloalkyl—optionally substituted with alkyl, alkoxy or halogen—or alternatively A₂ and B₂ form, together with the nitrogen atom which bears them, a mono- or polynuclear, preferably mono- or binuclear, saturated, unsaturated or aromatic heterocycle optionally substituted with alkyl, alkoxy or halogen), the groups forming thiourethane of the formula —CS—NA₂B₂ (in which A₂ and B₂ are as defined above), diacyl groups in which

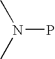

in the formulae III and IV represents the group

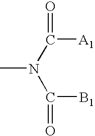

in which A₁ and B₁ are, independently, alkyl, aryl, arylalkyl or cycloalkyl—optionally substituted with alkyl, alkoxy or halogen, or alternatively A₁ and B₁ form, together with N and the two carbonyl groups, a mono- or polynuclear, preferably mono- or binuclear, saturated, unsaturated or aromatic heterocycle optionally substituted with alkyl, alkoxy or halogen—such as phthalimide, tetrahydropyranyl groups and, more rarely, alkyl, alkenyl (allyl or isopropenyl), arylalkyl such as trityl or benzyl, and groups of benzylidene type.

Examples of protecting groups for the amino group which may be mentioned are the formyl group, the acetyl group, the chloroacetyl group, the dichloroacetyl group, the phenylacetyl group, the thienylacetyl group, the tert-butoxycarbonyl group, the benzyloxycarbonyl group, the trityl group, the p-methoxybenzyl group, the diphenylmethyl group, the benzylidene group, the p-nitrobenzylidene group, the m-nitrobenzylidene group, the 3,4-methylene-dioxybenzylidene group and the m-chlorobenzylidene group.

Protecting groups that are particularly preferred are especially (C₁—C₆)alkoxycarbonyl and (C₆—C₁₀)aryl-(C₁—C₆)alkoxycarbonyl, such as tert-butoxycarbonyl and benzyloxycarbonyl.

The compounds of the formula II result of from the removal of the protecting group P in the corresponding compounds of the formula IV. The removal of the protecting group P is carried out in a manner which is conventional per se. Suitable methods are described in particular in Protective Groups in Organic Synthesis, Greene T. W. and Wuts P. G. M, published by John Wiley and Sons, 1991 and in Protecting Groups, Kocienski P. J, 1994, Georg Thieme Verlag.

The compounds of the formula IV are prepared simply by reacting the corresponding amine of the formula III with Y—CO-Z in which Y—CO-Z is a carboxylic acid (in which case Z represents —OH) or an activated form thereof.

When Y—CO—Z represents a carboxylic acid in activated form, preferred activating groups are those which are well known in the art, such as, for example, chlorine, bromine, azide, imidazolide, p-nitrophenoxy, 1-benzotriazole, N—O-succinimide, acyloxy and more particularly pivaloyloxy, (C₁—C₄ alkoxy)-carbonyloxy such as C₂H₅O—CO—O—, and dialkyl- or dicycloalkyl-O-ureide.

When Z=OH in Y—CO-Z, the reaction of Y—CO-Z with compound III is carried out in the presence of a coupling agent such as a carbodiimide, optionally in the presence of an activating agent such as hydroxybenzotriazole or hydroxysuccinimide with intermediate formation of dialkyl- or dicycloalkyl-O-ureides. Representative coupling agents are dicyclohexyl- and diisopropylcarbodiimides, carbodiimides that are soluble in an aqueous medium, or bis(2-oxo-3-oxazolidinyl)phosphonyl chloride.

According to one particularly advantageous embodiment, Y—CO—Z represents Y—CO—OH or Y—CO-hal in which hal is halogen and more particularly a chlorine atom.

When Y—CO-Z represents Y—CO-hal, it is desirable to perform the process in the presence of a mineral or organic base such as, for example, a hydroxide (such as an ammonium or alkali metal hydroxide), a carbonate (such as an alkali metal or alkaline-earth metal carbonate), an alkali metal alkoxide, an alkali metal amide, ammonia, triethylamine, tributylamine, pyridine or N-methyl-morpholine.

Another suitable base which may be used is a resin-supported base. Resins of this type are commercially available.

Mention may be made, for example, of N,N-(diisopropyl) aminomethylpolystyrene and morpholinomethylpolystyrene resins.

The molar ratio of the base to the compound of the formula III generally ranges between 1 and 10 equivalents and preferably between 1 and 5 equivalents. However, the reaction may be carried out in the presence of a large excess of base without harming the correct progress of the reaction.

The reaction is preferably carried out in a solvent.

In certain cases, the base may act as solvent. This is the case, for example, for pyridine.

As a variant, it is advantageous to select a polar aprotic solvent and, for example, a halogenated hydrocarbon such as methylene chloride, chloroform, carbon tetrachloride, dichloroethane, chlorobenzene or dichlorobenzene, dichloroethane being particularly preferred.

The compounds III and Y—CO-hal are preferably reacted together in stoichiometric amounts. Nevertheless, an excess of the acid halide may be used. Thus, the molar ratio of Y—CO-hal to compound III generally ranges between 1 and 2 and preferably between 1 and 1.5.

During the reaction of Y—CO-hal with compound III, the temperature will generally be maintained between −20° C. and 20° C. and preferably between −10° C. and 10° C., for example between 0° C. and 5° C.

When Y—CO-Z represents Y—CO—OH, it is particularly advantageous to use bis(2-oxo-3-oxazolidinyl)phosphonyl chloride as coupling agent, in a proportion of from 1 to 2 and preferably from 1 to 1.5 molar equivalents relative to compound III, while at the same time working in a polar aprotic solvent such as a halogenated hydrocarbon.

According to this embodiment (Y—COZ=Y—CO—OH), aliphatic halogenated hydrocarbons are preferred as solvents, dichloromethane being particularly suitable, and the presence of a base in the reaction medium is desirable. Advantageously, the base is chosen from those defined above. Preferably, the molar ratio of Y—CO—OH to compound III ranges between 1 and 2 and preferably between 1 and 1.5 and the reaction temperature is maintained between −20° C. and 20° C. and better still between −10° C. and 10° C., for example between 0° C. and 5° C.

Scheme 2 below more specifically illustrates a method for synthesizing compound III.

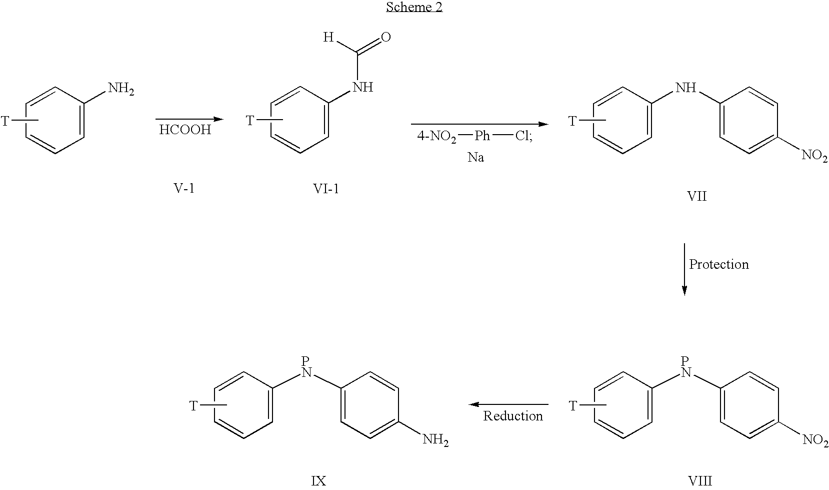

In this scheme, T and P as defined above for formula III.

In a first step, a suitable aniline of the formula V-1, in which T is as defined for compound Ill, is reacted with formic acid using an aromatic hydrocarbon such as toluene as solvent, so as to obtain the amide VI-1. This amide is reacted with p-nitrophenyl chloride in the presence of sodium in a mixture of amide and of aromatic hydrocarbon as solvent (such as a mixture of toluene and dimethylformamide), at a temperature of between 100° C. and 200° C., in the presence of a base (such as sodium hydroxide), to give the diphenylamine VII. After protecting the amino group of the amine VII, the resulting protected amine of the formula VIII is subjected to the action of a reducing agent so as to convert the nitro function into an amino function, to give the compound of the formula IX. This reduction is carried out, for example, by catalytic hydrogenation in the presence of palladium-on-charcoal.

A person skilled in the art will readily determine the precise reaction conditions for these reactions, these being conventional reactions of organic chemistry.

The compound of the formula VII may be prepared by reacting a suitable dihydroxyborane (compound V-2 below) with a suitable aniline (compound VI-2 below) according to the following reaction scheme:

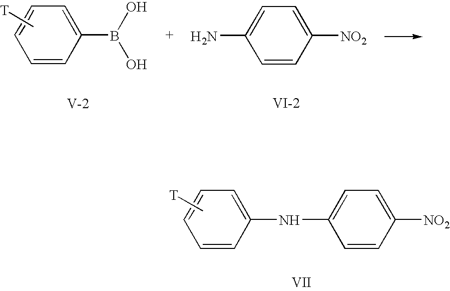

Suitable reaction conditions are:

the use of an aliphatic or aromatic halogenated hydrocarbon as solvent, such as methylene chloride, carbon tetrachloride, dichloroethane, chlorobenzene, dichlorobenzenes or chloroform, and mixtures thereof (preferably methylene chloride);

the presence of a base in the reaction medium and more particularly triethylamine, tributylamine, pyridine or 4-dimethylaminopyridine (and preferably triethylamine), and

the presence of cuprous diacetate (Cu(OAc)2) in the reaction medium,

a temperature of between 10° C. and 60° C. and preferably between 15° C. and 50° C.

Variant A2

This second variant allows the preparation of compounds of the formula II in which A represents CO and X represents methyl.

The synthetic steps are illustrated in Scheme 3.

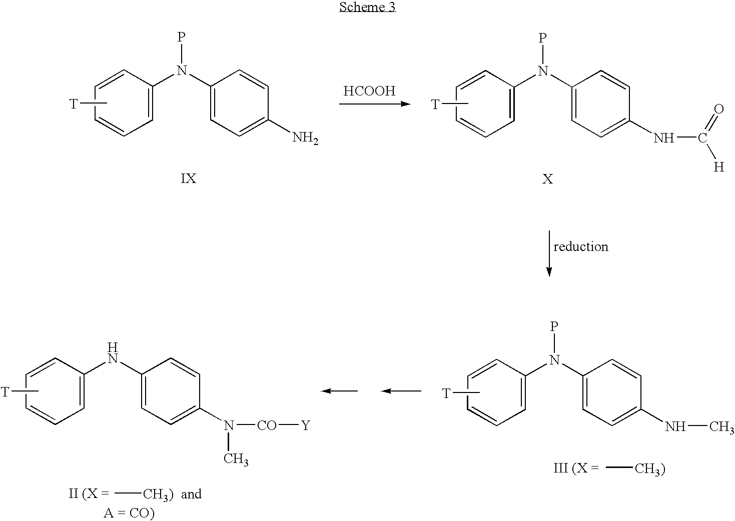

In this Scheme 3, T and P are as defined above for formula III and Y is as defined above for formula I.

In a first step, a suitable amine of the formula IX is treated with formic acid so as to form the amide of the formula X.

Formic acid and the amine IX are reacted together in stoichiometric amounts. Preferably, the molar ratio of formic acid to amine IX ranges between 1 and 1.5 and better still between 1 and 1.3.

This reaction can be carried out in a polar aprotic solvent such as an optionally halogenated aromatic hydrocarbon of the type such as benzene, toluene, xylene, chlorobenzene or dichlorobenzene. In a first stage, the reaction medium is maintained at a temperature of between 10° C. and 40° C., preferably between 20° C. and 30° C., for 5 to 48 hours, for example for 10 to 24 hours. Next, the mixture is heated to between 80° C. and 150° C. and preferably between 90° C. and 120° C. so as to bring about the removal of water, leading to the expected compound of the formula X.

In the next step, the amide formed X is reduced by the action of a suitable reducing agent to the amine of the formula Ill. Examples of suitable reducing agents are borane, trichlorosilane, dimethyl sulfide/borane (SMe₂, BH₃) and the system BF₃-Et₂O/NaBH₄. According to one preferred embodiment, excess dimethyl sulfide/borane is used. Thus, the molar ratio of dimethyl sulfide/borane to amide X will preferably be maintained between 1.5 and 5 equivalents and better still between 2 and 4 equivalents, for example between 2 and 3 equivalents.

The reduction is advantageously carried out in a polar aprotic solvent and more particularly in an ether, such as diethyl ether, diisopropyl ether, tetrahydrofuran, dioxane , dimethoxyethane or diethylene glycol dimethyl ether. Dimethoxyethane is the preferred solvent. The reaction medium is preferably maintained during the reduction at a temperature of between 40° C. and 100° C. and preferably between 50° C. and 80° C. After the reaction, the reaction medium is worked up in the usual manner.

The reaction medium is conventionally cooled to between −10° C. and 10° C. and water is then added to the medium, after which the amine formed of the formula III is isolated.

The conversion of the compound of the formula III into a compound of the formula II is carded out by performing the process as in Scheme 1 by reaction with a suitable carboxylic acid or an activated derivative thereof of the formula Y—CO—Z, followed by deprotection of the amino function of the resulting diphenylamine.

B—Preparation of the Compounds of the Formula II in Which A Represents CO and X Represents -A—Y.

These compounds may be prepared by following the reaction scheme illustrated in Scheme 4:

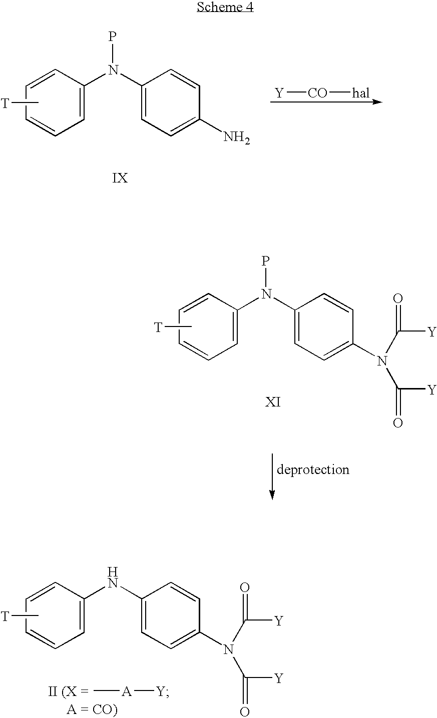

In this scheme, Y and T are as defined above for formula II and P is as defined above for formula III.

The reaction of compound IX with the acid halide of the formula Y—CO-hal in which Y is as defined in formula II and hal represents a halogen atom, preferably a chlorine atom, is carried out in a manner which is conventional per se, under conditions similar to those used for the reaction of compound III with Y—CO—Z when Z represents hal (Scheme 1), except that it is necessary to perform the process in the presence of at least two equivalents of Y—CO-hal per equivalent of compound of the formula IX.

Usually, the molar ratio of Y—CO-hal to compound IX ranges between 2 and 10, for example between 2 and 5 and better still between 2 and 4.

According to one particularly preferred embodiment of the invention, the reagent Y—CO-hal, dissolved in the solvent, is added per portion of about 1 molar equivalent to the reaction medium. At each addition of Y—CO-hal, a simultaneous addition of base is carried out. At each addition, the molar ratio of the base to the reagent Y—CO-hal is maintained between 1.5 and 10 and preferably between 1.5 and 3. Overall, the amount of amine used in the reaction ranges between 1.5 and 10 equivalents and preferably between 1.5 and 3 equivalents per mole of Y—CO-hal.

Preferred operating conditions are the use of triethylamine as base, the use of dichloromethane as solvent and a temperature of between 10° C. and 50° C. and preferably between 20° C. and 30° C. during the addition of the reagents, followed by a temperature of 30° C. to 80° C. and preferably from 30° C. to 50° C. for 30 minutes to 15 hours (for example for 1 to 5 hours) after addition of the reagents.

Compound XI is then subjected to a deprotection reaction so as to remove the protecting group P from the amino function. This reaction is carried out in a manner which is conventional per se.

C—Preparation of the Compounds of the Formula II in Which A Represents SO₂ and X Represents H or Alkyl.

Scheme 5 illustrates one route for preparing these compounds.

A suitable amine of the formula III is reacted with a sulfonyl halide of the formula Y—SO₂-hal in which Y is as defined in formula II and hal represents a halogen atom, preferably a chlorine atom. The molar ratio of the sulfonyl halide to the compound of the formula XI preferentially ranges between 1 and 2 and preferably between 1 and 1.5.

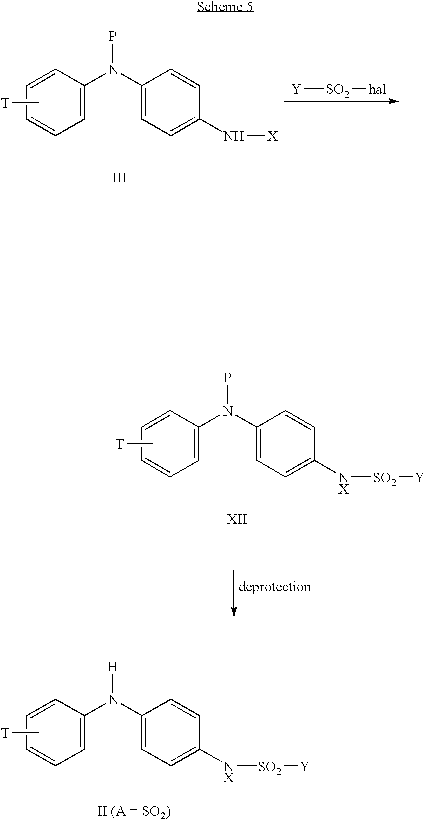

In this scheme, T and P are as defined above for formula III, Y is as defined for formula I and X represents H or (C₁—C₁₀)alkyl.

The reaction is preferably carried out at between −10° C. and 10° C., for example between −5° C. and 5° C., in the presence of a base as defined above in the case of the reaction of compound III with Y—CO—Z (Scheme 1).

The preferred bases are pyridine and triethylamine. Advantageously, the molar ratio of the base to Y—SO₂-hal ranges between 1.5 and 10, preferably between 1.5 and 5 and better still between 1.5 and 3.

As a variant, the base may act as solvent, in which case it is present in very large excess in the reaction medium.

One example of a base which is suitable as solvent is pyridine.

When the reaction of III with YO₂-hal is carried out in a solvent which is different from the base, this solvent is preferably a polar aprotic solvent chosen from an aliphatic or aromatic halogenated hydrocarbon (such as methylene chloride, chloroform, carbon tetrachloride, dichloroethane, chlorobenzene or dichlorobenzene), an ether (such as diethyl ether, diisopropyl ether, tetrahydrofuran, dioxane, dimethoxyethane or diethylene glycol dimethyl ether), a nitrile (such as acetonitrile or isobutyronitrile) or an amide (such as formamide, dimethylformamide, dimethylacetamide, N-methylpyrrolidone or hexamethylphosphoramide).

The reaction temperature is advantageously maintained between −30° C. and 50° C. and preferably between −10° C. and 10° C.

The compound of the formula XII is thus isolated.

This compound is treated in a conventional manner so as to deprotect the amino function by removal of the protecting group P.

D—Preparation of the Compounds of the Formula II in Which A Represents SO₂ and X Represents -A-Y.

These compounds are prepared simply by following the reaction scheme given in Scheme 6:

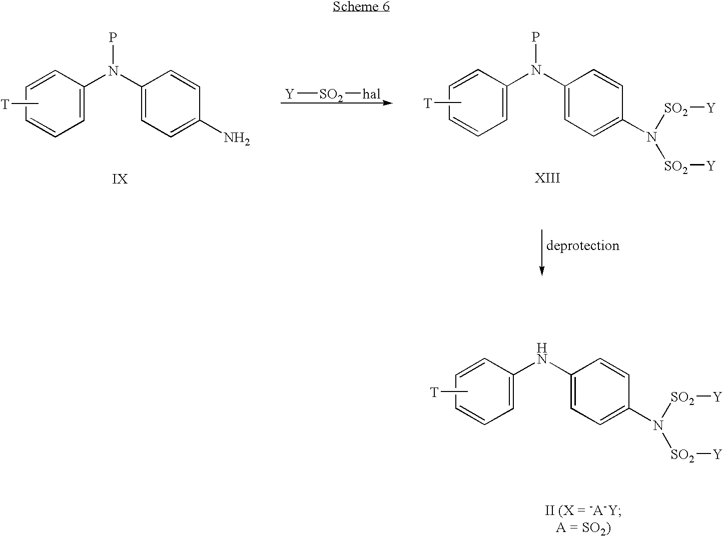

In this scheme, T and P are as defined above for formula III and Y is as defined above for formula I.

In order to synthesize the compound of the formula XIII, it is necessary to react at least 2 equivalents of the sulfonyl halide of the formula Y—SO₂-hal with the compound of the formula IX. Preferably, the molar ratio of Y—SO₂-hal to compound IX ranges between 2 and 10 and preferably between 2 and 5, for example between 2 and 4.

The reaction of compound IX with Y—SO₂hal is carried out in the presence of a base; the bases which may be used are those recommended for the reaction of compound III with Y—SO₂hal (Scheme 5). The molar ratio of the base to Y—SO₂-hal is preferably between 2 and 10 and better still between 2 and 5, for example between 3 and 4. A preferred base which may be mentioned is triethylamine. The reaction of compound IX with Y—SO₂-hal is either carried out using the base as solvent, for example pyridine, or in one of the preferred solvents recommended in the case of the reaction of compound III with Y—SO₂-hal (Scheme 5) and more particularly in an aliphatic halogenated hydrocarbon such as dichloromethane.

According to one preferred embodiment of the invention, Y—SO₂-hal and the base are added portionwise to a solution of compound IX in a solvent. At each addition, from 1 to 1.5 equivalents of Y—SO₂-hal relative to compound IX and from 1 to 1.5 equivalents of base relative to compound IX are added.

The reaction of compound IX with Y—SO₂-hal is preferably carried out at a temperature of between 10° C. and 50° C. and better still between 20° C. and 30° C.

E—Preparation of the Compounds of the Formula II in Which A Represents —CO—NRa— and X Represents H or Alkyl.

The compounds of the formula II in which Ra represents H are prepared, for example, by carrying out the process illustrated in Scheme 7.

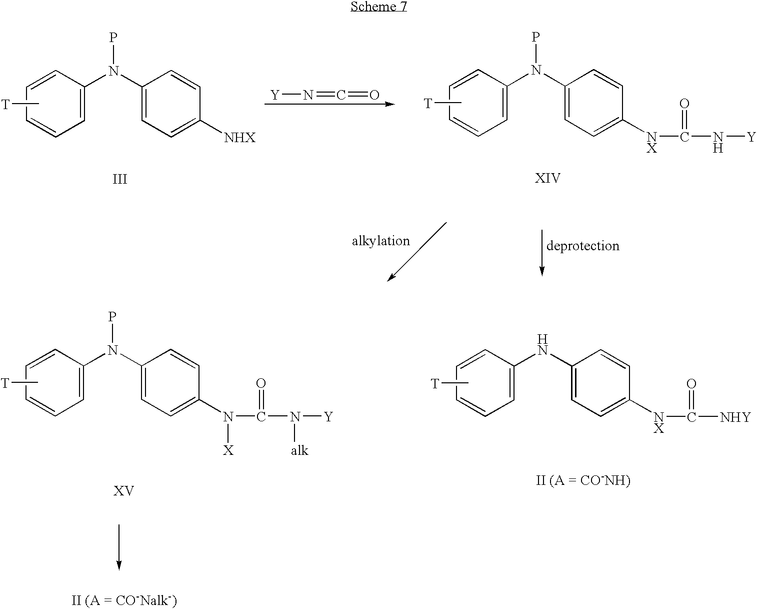

In this scheme, T and P are as defined above for formula III, Y is as defined above for formula I, X represents H or (C₁—C₁₀)alkyl and alk represents (C₁—C₁₀)alkyl.

In a first step, the compounds of the formula XIV are prepared by reacting a compound of the formula III with an isocyanate of the formula Y—N=C=O. The process is preferably performed in a solvent, at a temperature of between 10° C. and 50° C., in the presence of 0.8 to 1.3 equivalents of isocyanate relative to the compound of the formula III.

According to one preferred embodiment of the invention, the molar ratio of the isocyanate to compound III ranges between 0.8 and 1, and the reaction temperature is between 20° C. and 30° C.

The preferred solvents are polar aprotic solvents of the aliphatic or aromatic halogenated hydrocarbon type, such as methylene chloride, chloroform, carbon tetrachloride, dichloroethane, chlorobenzene or dichlorobenzene. Dichloromethane is clearly preferred.

The expected compound of the formula XIV is isolated at the end of the reaction.

The compounds of the formula II in which Ra represents H are readily obtained from the compounds of the formula XIV, by deprotection of the amino function, by means of removing the group P in a conventional manner.

The compounds of the formula II in which Ra represents alkyl are similarly obtained from the compounds of the formula XV by deprotection of the amino function, by means of removing the group P.

The compounds of the formula XV may be simply prepared from the corresponding compounds of the formula XIV by alkylating the amino function, for example by the action of an alkyl halide in the presence of a base. When X represents H in the compound of the formula XIV, and in order to prepare a compound of XV in which X represents H, it is necessary first to protect the amino function directly linked to the phenyl nucleus before carrying out the alkylation of the amino function of the group —NH—Y. After alkylation, this protective function is removed conventionally to give the desired compound XV.

After deprotection, compound XV gives the compound of the formula II in which A represents —CO—NRa- in which Ra represents alkyl, X being either H or alkyl.

F—Preparation of the Compounds of the Formula II in Which A Represents —CO—NRa—SO₂— and X represents H or alkyl

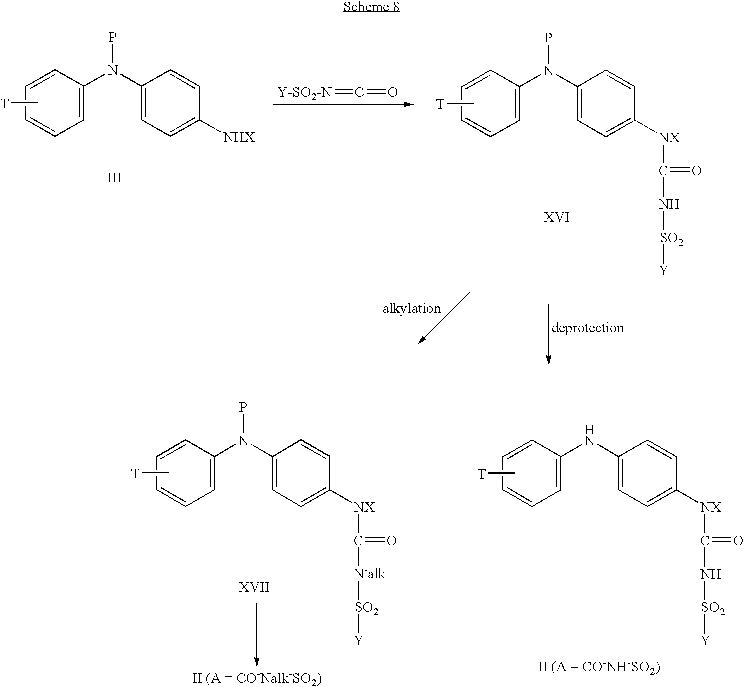

In this scheme, T and P are as defined above for formula III, Y is as defined above for formula I, X represents H or (C₁—C₁₀) alkyl and alk represents (C₁—C₁₀)alkyl.

In a first step, the compound of the formula III is reacted with a sulfonyl isocyanate of the formula Y—SO₂—N=C=O. The reaction conditions recommended for this reaction are those generally described above for the reaction of compound III with the isocyanate Y—N=C=O (Scheme 7).

The solvent is preferably an aliphatic or aromatic halogenated hydrocarbon as described above, more particularly dichloromethane.

Advantageously, from 1 to 1.3 equivalents of the sulfonyl isocyanate are used relative to the compound of the formula III.

It is preferable to perform the process in the presence of a base, such as triethylamine and more generally such as one of those described above.

Compound XVI is obtained at the end of this step.

After deprotection of the amino function of compound XVI, a compound II is isolated in which Ra represents a hydrogen atom.

As a variant, compound XVI may be subjected to an alkylation reaction before deprotection of the amino function, which leads to the intermediate compound of the formula XVII.

When X represents H in compound XVI, and in order to prepare a compound XVII in which X represents H, it may be necessary, before alkylation, to temporarily protect the amino function directly linked to the phenyl nucleus of the diphenylamine XVI. Immediately after alkylation, the amino function is deprotected, which gives the desired compound XVII.

This compound is subjected to a deprotection reaction to obtain the expected compound of the formula II in which A represents —CO—NRa—SO₂— in which Ra represents alkyl.

G—Preparation of the Compounds of the Formula II in Which A Represents —CO—, —SO₂— or —CO—NRₐ— (preferably —CO— or SO₂) and X represents H or alkyl.

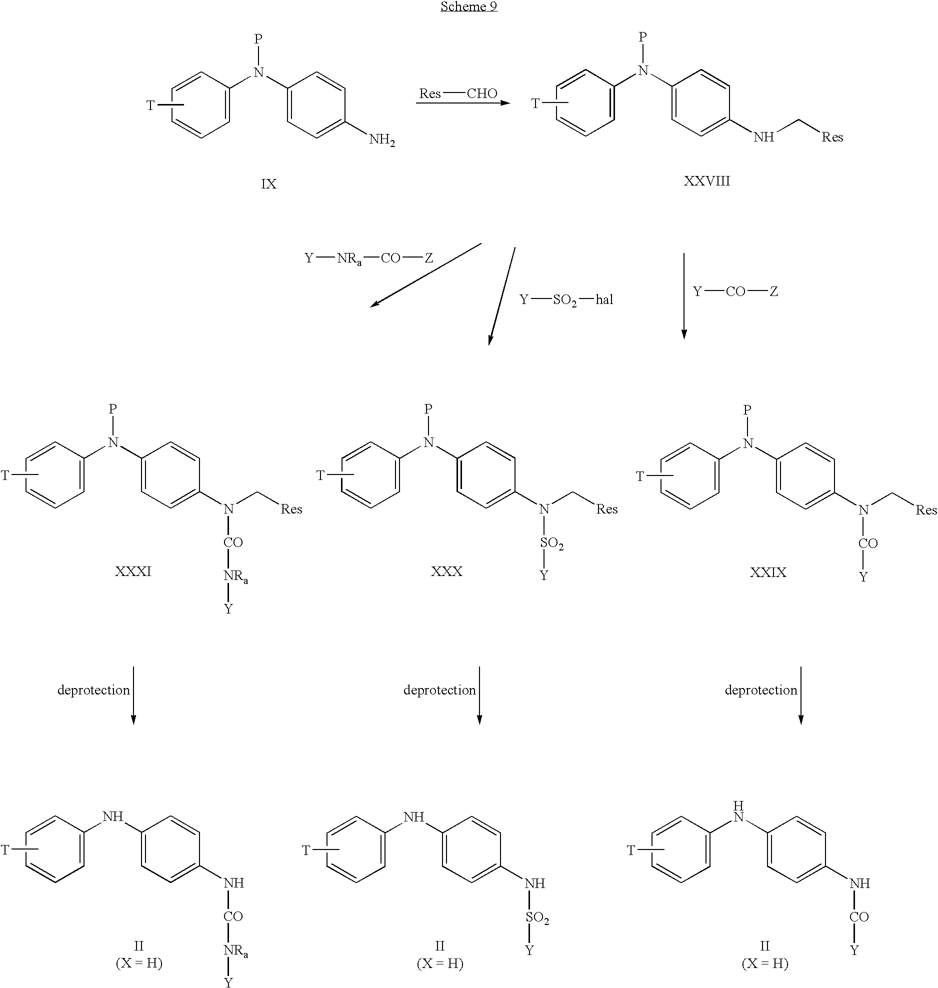

In the above scheme, Y—SO₂-hal is as defined above for Scheme 5; Y—CO—Z is as defined above for Scheme 1; and in Y—NRₐ—CO—Z′, Y and Rₐ are as defined above for formula I and Z′ has one of the meanings given above for Z (Scheme 5). Res-CHO denotes a resin functionalized with formyl groups.

In a first step, compound IX is reacted with the resin under conditions suitable for promoting the reductive amination reaction of a formyl group of the resin. These conditions vary according to the type of resin used.

Advantageously, a solution of compound IX and of the resin is prepared in an aliphatic halogenated hydrocarbon in the presence of a protic acid such as acetic acid, and a hydride such as sodium triacetoxyborohydride is then added to this mixture.

In a second step, the resulting functionalized resin is reacted with Y—CO—Z, Y—SO₂-hal or Y—NRₐ—CO—Z′ under the conventional conditions and in particular in the presence of a base such as that generally recommended above for Scheme 1.

When Z or Z′ represents OH, it is desirable to perform the process in the presence of a coupling agent (such as bis(2-oxo-3-oxazolidinyl)phosphonyl chloride as described above for Scheme 1.

In a third step, the secondary amine of the diphenylamine is the protected and the resin is removed. This is generally carried out by the action of a strong acid such as trifluoroacetic acid. The appropriate conditions depend both on the nature of the resin used and on the type of protecting group present in the molecule.

As a type of resin which may be used, mention may be made of the resin Argopore MB—CHO sold by the company Argonaut.

After step 3, a compound of the formula II is isolated in which X=H, which is readily converted into a compound II in which X=alkyl, conventionally by selective protection of the secondary amine function of the diphenylamine, followed by alkylation and finally deprotection of the amine function.

Some of the compounds of the formula II:

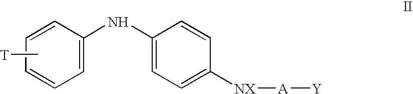

in which T, X, A and Y are as defined in formula I above are novel. They form an integral part of the invention.

A first subgroup of novel compounds II consists of the compounds in which:

T represents —OCH₃; A represents SO₂; X represents H; and Y is as defined above for formula I, on condition that Y does not represent unsubstituted phenyl.

A second subgroup of novel compounds II consists of the compounds in which:

T represents —OCH₃; X represents H; A represents CO; and Y is as defined above for formula I, on condition that Y does not represent methyl; phenyl; ethyl; 2-haloethyl; ethoxy; 2-mercaptoethyl; vinyl; 1-methylvinyl; 3-amino-3-carboxypropyl; morpholinyl; phenoxy; and benzyloxy.

A third subgroup of novel compounds II consists of the compounds in which:

T represents —CF₃; or alkylthio and, for example, —SCH₃; X represents H and A and Y are as defined above for formula I. Among these compounds, those for which A represents CO or SO₂ are preferred.

A fourth subgroup of novel compounds of the formula II consists of the compounds in which:

T represents —OCH₃; X represents H or alky, for example (C₁—C₆)alkyl such as methyl, ethyl, n-propyl, isopropyl, isobutyl, n-butyl or tert-butyl, but preferably H; and Y represents pyridyl optionally substituted with one or more radicals R as defined above for formula I and A is as defined above for formula I.

The preferred substituents R are such as those defined above.

Among these compounds, those for which X represents H are preferred.

A fifth subgroup of novel compounds II consists of the compounds in which T represents H; X represents -A-Y; A represents CO; and Y represents furyl optionally substituted with one or more radicals R as defined above for formula I. The preferred substituents R are such as those defined above.

A sixth subgroup of novel compounds II consists of the compounds in which T represents H; X represents H; A represents —CO—NH—SO₂—; and Y represents phenyl optionally substituted with one or more radicals R as defined above for formula I. The preferred substituents R are such as those defined above.

A seventh subgroup of novel compounds II consists of the compounds in which T represents a hydrogen atom; A represents CO; X represents H; Y represents benzyl optionally substituted on the phenyl nucleus with one or more radicals R, with the exclusion of amino and nitro radicals; or alternatively Y represents —CH₂—Cy¹ in which Cy¹ is heteroaryl (with the exclusion of 2-pyridyl) optionally substituted with one or more radicals R. Preferably, R is a radical G or an alkoxycarbonyl group as generally defined for formula I. Preferred meanings of Y are 3-pyridylmethyl and 3,5-di-t-butyl-4-hydroxybenzyl.

An eighth subgroup of novel compounds II consists of the compounds in which T represents a hydrogen atom; A represents CO; X represents H; and Y represents phenyl substituted with one or more radicals chosen from nitro and optionally halogenated alkyl (such as CF₃).

A ninth subgroup of novel compounds II consists of the compounds in which T represents a hydrogen atom; A represents CO; X represents H; Y represents idolyl (such as 3-idolyl) or pyrazinyl (such as 2-pyrazinyl), indolyl and pyrazinyl optionally being substituted with one or more oxo radicals and/or radicals R as defined above.

A tenth subgroup of novel compounds II consists of the compounds in which T represents a hydrogen atom; A represents SO₂; X represents H; and Y represents a group —CH=CH—Cy⁰ in which Cy⁰ is phenyl optionally substituted with one or more radicals R; benzyl optionally substituted with one or more radicals R; heteroaryl (with the exclusion of benzopyran and coumarin) optionally substituted with one or more oxo radicals and/or radicals R as defined above; phenyl substituted with one or more radicals chosen from a fluorine atom, CF₃, OCF₃ and cyano.

An eleventh group of novel compounds consists of the following compounds:

1) T=H; A=CO—NH—SO₂; Y=phenyl;

2) T=H; X=AY; A=CO; Y=cyclopentyl;

3) T=H; X=AY; A=SO₂; Y=phenyl;

4) T=4—OCH₃; X=CH₃; A=CO; Y-3-pyridyl;

5) T=H; A=CO; X=H; Y=2-methoxyphenyl;

6) T=H; A=CO; X=H; Y=2,4-dimethoxyphenyl;

7) T=H; A=CO; X=H; Y=2-pyridyl or 4-pyridyl or 2-furyl or 2,6-dimethoxy-3-pyridyl or 3-pyridyl N-oxide.

For each of the subgroups of compounds of the formula II defined above, preferred meanings of R and G are those listed above in the case of the formula I.

Not only may the compounds of the formula II above be used as intermediates in the synthesis of the compounds of the formula I, but also they have antioxidant activity which makes them capable of limiting the destructive activity of oxidative free-radical species.

The antioxidant activity of the compounds of the formula II is revealed in vitro, for example, by evaluating the ability of the compounds of the formula II to prevent the oxidation of human low molecular weight lipoproteins.

In the test carried out, the human low molecular weight lipoproteins are oxidized with cupric ions for 24 hours at 37° C. The B apoprotein bome by these lipoproteins becomes fluorescent on oxidation (excitation at 360 nm, emission at 460 nm). In the presence of the compounds of the formula II, a decrease in fluorescence is noted, which reflects the antioxidant power of the compounds of the formula II. The results are expressed in the form of a 50% inhibitory concentration (IC₅₀). The IC₅₀ values measured in the case of a certain number of compounds of the formula II are given in the following table:

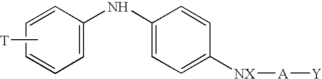

| T | X | A | Y | IC₅₀ (μM) |
|---|---|---|---|---|
| 4-MeO— | H | CO | 3-pyridyl | 3.8 |
| H | H | CO | 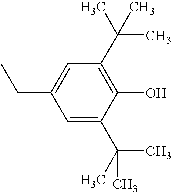 | 1.7 |
| 4-Me—O— | H | CO | 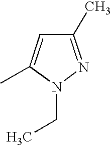 | 8.3 |
| 4-Me—O— | H | CO | 5-pyrimidyl | 5.2 |
| H | H | CO | 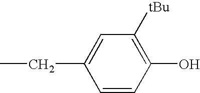 | 4.8 |
| 4-Me—O— | H | SO₂ | 3-pyridyl | 7.9 |
| 4-Me—O— | H | CO | 3-pyridyl N-oxide | 10.5 |
| 4-Me—O— | H | CO | 3-NO₂—C₆H₄— | 6.5 |
| 4-MeS— | H | CO | C₆H₅ | 1.8 |
| H | H | SO₂ | 3-pyridyl | 7.4 |
| H | H | SO₂ | 2-thienyl | 3.3 |
| H | H | SO₂ | 3-F—C₆H₄— | 3.7 |
| H | H | SO₂ | 4-CN—C₆H₄ | 11.8 |
| H | H | SO₂ | 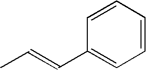 | 12.2 |
| H | H | SO₂ | 4-CH₃CONH—C₆H₄— | 12.3 |
| H | H | SO₂ | 3-Cl,4-CH₃CONH—C₆H₃— | 13.4 |
| H | H | SO₂ | 4-NO₂—C₆H₄ | 4.3 |
| 4-MeO— | H | CO | 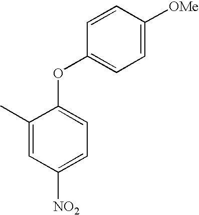 | 8.8 |
| 4-MeO— | H | CO | 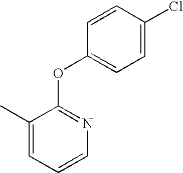 | 3.6 |
| 4-MeO— | H | CO | 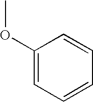 | 2.4 |
| 4-MeO— | H | CO | 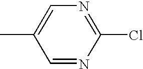 | 2.6 |
| 4-MeO— | H | CO | 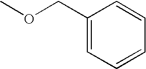 | 2 |
| 4-MeO— | H | CO | 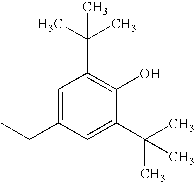 | 2.8 |
| 4-MeO— | H | CO | 4-(CH₃)₂N—C₆H₄— | 11.2 |
| 4-MeO— | H | SO₂ | 4-MeO—C₆H₄— | 5 |
| 4-MeO— | H | CO | 2-furyl | 8.4 |
| 4-MeO— | H | CO | 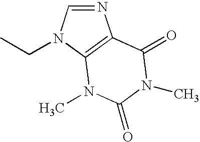 | 7.7 |
| 4-MeO— | H | CO | 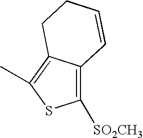 | 7.3 |
| 4-MeO— | H | CO | 3-CF₃—C₆H₄— | 6.4 |
| 4-MeO— | H | CO | 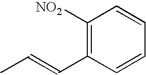 | 13.3 |
| 4-MeO— | H | CO | 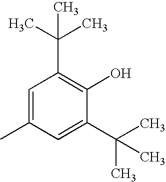 | 9.4 |
| 4-MeO— | H | CO | 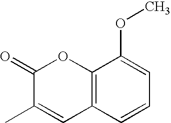 | 8.7 |
| 4-MeO— | H | CO | 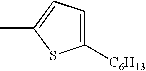 | 11.1 |
| 4-MeO— | H | CO | 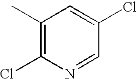 | 5.4 |
| 4-MeO— | H | SO₂ | 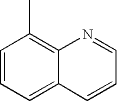 | 8.7 |

According to another of its aspects, the invention relates to a pharmaceutical composition comprising at least one compound of the formula I as defined above, in combination with at least one pharmaceutically acceptable excipient.

According to yet another of its aspects, the invention relates to a pharmaceutical composition comprising at least one compound of the formula II, in combination with at least one pharmaceutically acceptable excipient.

These compounds may be administered orally in the form of tablets, gel capsules or granules with immediate release or controlled release, intravenously in the form of an injectable solution, transdermally in the form of an adhesive transdermal device, or locally in the form of a solution, cream or gel.

A solid composition for oral administration is prepared by adding to the active principle a filler and, where appropriate, a binder, a crumbling agent, a lubricant, a colorant or a flavour corrector, and by shaping the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatin, shellac, hydroxypropylcellulose, hydroxypropylmethyl-cellulose, calcium citrate, dextrin and pectin. Examples of lubricants include magnesium stearate, talc, polyethylene glycol, silica and hardened plant oils. The colorant may be any colorant permitted for use in medicinal products. Examples of flavour correctors include cocoa powder, mint in herb form, aromatic powder, mint in oil form, bomeol and cinnamon powder. Needless to say, the tablet or granulate may be suitably coated with sugar, gelatin or the like.

An injectable form containing the compound of the present invention as active principle is prepared, where appropriate, by mixing the said compound with a pH regulator, a buffer agent, a suspending agent, solubilizing agent, a stabilizer, a tonicity agent and/or a preserving agent, and by converting the mixture into an a form for intravenous, subcutaneous or intramuscular injection, according to a conventional process. Where appropriate, the injectable form obtained may be freeze-dried by a conventional process.

Examples of suspending agents include methylcellulose, polysorbate 80, hydroxyethylcellulose, acacia, powdered gum tragacanth, sodium carboxymethylcellulose and polyethoxylated sorbitan monolaurate.

Examples of solubilizing agents include castor oil solidified with polyoxyethylene, polysorbate 80, nicotinamide, polyethoxylated sorbitan monolaurate and the ethyl ester of castor oil fatty acid.

In addition, the stabilizer encompasses sodium sulfite, sodium metasulfite and ether, while the preserving agent encompasses methyl p-hydroxybenzoate, ethyl p-hydroxybenzoate, sorbic acid, phenol, cresol and chlorocresol.

In this context, the substances according to the invention are as a rule preferably administered in doses between approximately 0.1 and 100 mg, in particular between 1 and 10 mg, per dose unit. The daily dose is preferably between approximately 0.001 and 10 mg/kg of body weight. The specific dose for each patient, however, depends on all sorts of factors, for example on the efficacy of the specific compound employed, on the age, body weight, general state of health, sex, on the diet, on the time and route of administration, on the excretion rate, pharmaceutical combination and severity of the particular disorder to which the therapy applies. Oral administration is preferred.

According to another of its aspects, the invention relates to the use of a compound of the formula I as defined above, for the preparation of a medicinal product for treating pathologies characterized by a lack of nitrogen monoxide production and/or an oxidative stress condition.

According to another of its aspects, the invention relates to the use of a compound of the formula I as defined above, for the preparation of a medicinal product which may be used in the treatment of

a atherosclerosis-associated ischaemias (lipid peroxidation, development, progress and rupture of atheroma plaques, platelet activation);

restenosis after angioplasty;

stenosis after vascular surgery;

diabetes;

insulin resistance;

retinal and renal microvascular complications of diabetes;

the cardiovascular risk of diabetes in so far as it is not explained by the conventional factors;

male erectile dysfunction;

cerebral hypoxia;

chronic rejection after organ transplantation;

articular pathologies.

According to one of its final aspects, the invention relates to the use of a compound of the formula II for the preparation of an antioxidant medicinal product which may be used as a free-radical scavenger.

The present invention is illustrated hereinbelow in the light of the examples which follow.

The frequency of the MMR apparatus used to record the proton spectra in the examples given below is 300 MHz.

Examples of compounds I are, in particular, those illustrated in Tables 1 to 7 below.

### EXAMPLE 1

Compound of the formula I: T=4-OCH₃; X=H; A=CO; Y=3-pyridyl.

Step a:

Compound of the formula IV: T=4-OCH₃; P=C₄H₉ OCO—; X=H; Y=3-pyridyl

56.1 g (315 mmol) of nicotinoyl chloride hydrochloride are added to a mixture of 99 g (315 mmol) of 4amino4′-methoxy-N-butoxycarbonyl-diphenylamine and 95.6 g (945 mmol) of triethylamine in 3.15 I of dichloroethane.

After stirring for 3 hours at room temperature, 31.9 g (315 mmol) of triethylamine and 56.1 g (315 mmol) of nicotinoyl chloride hydrochloride are added.

After stirring for 42 hours at room temperature, the reaction medium is called into 3 I of ice-cold water and extracted with dichloromethane (2×1 I).

The organic phase is washed with water (3×500 ml), dried over Na₂SO₄ and concentrated.

The oil obtained is purified by flash chromatography on silicone gel, with an ethyl acetate/heptane mixture (1:1) followed by dichloromethane/methanol (98:2), to give 132.2 g of a light brown pasty solid (quantitative yield).

NMR (CDCl₃) δ (ppm): 1.35 (9 H, s); 3.7 (3 H, s); 6.8 (2 H, d, J=9 Hz); 7.0 (4 H, m); 7.25 (1 H, m); 7.4 (2 H, m); 8.0 (1 H, m); 8.6 (1 H, m); 8.85 (1 H, s) ; 8.95 (1 H, s).

Step b:

Compound of the formula II: T=4-OCH₃; A=CO; Y=3-pyridyl; X=H

A mixture of 132.2 g (315 mmol) of the compound obtained in step a) and 945 ml of trifluoroacetic acid is stirred for 16 hours at room temperature and then poured onto 1.8 kg of ice.

The medium is basified by slow addition of 3.5 I of 10% NaOH solution, while keeping the temperature between 20° C. and 25° C. by addition of ice.

About 1 litre of dichloromethane is added, after which the precipitate formed is filtered off, rinsed with Et₂O (3×300 ml) and dried under vacuum in the presence of P₂O₅.

96.8 g of a light brown solid are obtained (yield=95.2%).

M.p.=190-192° C.

NMR (CDCl₃) δ (ppm): 3.7 (3 H, s); 6.75-6.9 (4 H, m); 7.0 (2 H, m); 7.35 (3 H, m); 7.65 (1 H, m); 8.1 (1 H, m); 8.65 (1 H, m); 9.0 (1 H, s).

Step c:

Title compound of the formula I.

A solution of 20.5 g (298 mmol) of sodium nitrite in 1 125 ml of water is added dropwise, while maintaining at 20° C., to a solution of 47.9 g (149 mmol) of the compound obtained in step b in 1 125 ml of acetic acid.

After stirring for 1 hour, the reaction medium is poured onto a mixture of 1 500 g of NaHCO₃, 9 I of water and 2 kg of ice.

The precipitate formed is filtered off and dried under vacuum in the presence of P₂O₅ at room temperature, to give 45.6 g of a pink solid (yield=88%).

M.p.=148-150° C.

NMR (CDCl₃) δ (ppm): 3.8 (3 H, 2 s); 6.9-7.1 (4 H, m); 7.3-7.5 (3 H, m); 7.65-7.8 (2 H, m); 8.0-8.2 (2 H, m); 8.8(1 H, m); 9.0 (1 H, 2 s).

### EXAMPLE 2

Preparation of the Compound of the Formula II: T=H; A=CO; Y=—C₆H₅; X=H

0.3 g (2.1 mmol) of benzoyl chloride is added to a mixture of 0.6 g (2.1 mmol) of 4-amino-N-butoxycarbonyldiphenylamine in 20 ml of pyridine.

The reaction medium is reflux for half an hour.

After cooling, a mixture of ice +HCl is added.

The precipitate formed is filtered off, spin-filtered and dried to give 0.8 g of a grey solid (quantitative yield).

M.p.=130-132° C.

NMR (CDCl₃) δ (ppm): 1.35 (9 H, s); 7.0-7.3 (7 H, m, including 1 H exchangeable with CF₃COOD); 7.3-7.6 (6 H, m); 7.7-7.85 (2 H, m).

### EXAMPLE 3

Preparation of the Compound of the Formula II: T=4-OCH₃; A=CO, Y=5-pyrimidinyl; X=H

0.592 g (4.8 mmol) of 5-pyrimidinecarboxylic acid hydrochloride and then 1.21 g (4.0 mmol) of bis(2-oxo-3-oxazolidinyl)phosphonyl chloride are added to a mixture, maintained at 0° C., of 1.5 g (4.8 mmol) of 4-amino-4′-methoxy-N-butoxycarbonyldiphenylamine and 1.33 ml (9.6 mmol) of triethylamine in 145 ml of dichloromethane.

The mixture is allowed to return to room temperature and is stirred for 20 hours, after which it is refluxed for 6 hours.

The medium is then poured into cold water and extracted with dichloromethane.

The organic phase, washed with saturated NaHCO₃ solution and then with water to pH 7, is dried over Na₂SO₄ and then concentrated.

The residue obtained is purified by flash chromatography in a mixture of heptane/ethyl acetate (1:1) followed by dichloromethane/ethyl acetate (1:1) to give I g of a beige-coloured solid (yield=50%).

NMR (CDCl₃) δ (ppm): 1.3 (3 H, s); 3.7 (3 H, s); 6.85 (2 H, m); 7.05 (4 H, m); 7.3 (2 H, d, J=8.7 Hz); 8.35 (1 H, broad s); 9.1 (2 H, s); 9.2 (1 H, s).

### EXAMPLE 4

Compound of the Formula I: T=4-OCH₃; A=CO; X=H; Y=5-pyrimidinyl.

11 ml of ethanolic 15% ethyl nitrite solution are added to a solution, maintained between 0° C. and 5° C., of 0.56 g (1.75 mmol) of 4-methoxy4′-[5-pyrimidinylcarbonylamino]diphenylamine in 39 ml of chloroform and 10 ml of acetonitrile.

After stirring for 3 hours between 0° C. and 5° C., the reaction medium is concentrated under vacuum.

After washing the residue with heptane and drying under vacuum, 0.463 g of reddish crystals is obtained (yield=75.8%).

NMR (DMSO) δ (ppm): 3.6 (3 H, 2 s); 6.8-7.0 (4 H, m); 7.1 -7.25(2 H, m); 7.7(2 H, m); 9.1 (2 H, s); 9.2(1 H, 2 s); 10.6(2 H, 2 s).

### EXAMPLE 5

Compound of the Formula I: T=H; A=CO; X=—CO-2-furyl; Y=2-furyl.

Step a:

Compound of the formula XI: T=H; Y=2-furyl; P=—CO—O—C₄H₉

A mixture of 0.68 g (5.2 mmol) of 2-furoyl chloride and 10 ml of dichloromethane is added to a mixture of 1.5 g (5.2 mmol) of 4-amino-N-butoxycarbonyidiphenylamine, 1.6 g (15.6 mmol) of triethylamine and 40 ml of dichloromethane.

After stirring for 16 hours at room temperature, 1 g (10.4 mmol) of triethylamine and 0.68 g (5.2 mmol) of 2-furoyl chloride are added, followed, five hours later, by addition of a further 0.68 g (5.2 mmol) of 2-furoyl chloride, after which the mixture is left stirring for 16 hours.

The reaction medium is then maintained at reflux for 4 hours, after which it is poured into a water/HCl mixture and extracted with dichloromethane.

The organic phase, rinsed with water to neutral pH and dried over Na₂SO₄, is concentrated under vacuum.

The residue, taken up in 35 ml of pyridine, is placed in contact with 0.68 g (5.2 mmol) of 2-furoyl chloride and refluxed for half an hour.

The reaction medium is poured into an ice +HCl mixture.

The precipitate formed is rinsed with water and dried to give 1.86 g of a brown solid (yield=76%).

M.p.=67-71° C.

NMR (CDCl₃) δ (ppm): 1.4 (9 H, s); 6.3 (2 H, m); 6.9-7.45 (13 H, m).

Step b:

Compound of the formula II: T=H; A=CO; X=—CO-2-furyl; Y=2-furyl

This compound is obtained by carrying out the process as in Example 1, step b) starting with the compound obtained in the preceding step a) (yield=95%).

M.p.=152-156° C.

NMR (CDCl₃) δ (ppm): 5.8 (1H, broad s, exchangeable with CF₃COOD); 6.4 (2H, m); 6.8-7.6 (13H, m).

Step c:

Compound of the formula I: T=H ; A=CO ; X=—CO-2-furyl; Y=2-furyl

This compound is obtained by performing the process as in Example 1, step c) starting with the compound obtained in the preceding step b) (yield=97%).

M.p.=146-150° C.

NMR (CDCl₃) δ (ppm): 6.4 (2H, m); 6.9-7.15 (4H, m); 7.15-7.55 (9H, m)

### EXAMPLE 6

Compound of the formula I: T=H; A=SO₂; X=H; Y=3-pyridyl

Step a:

Compound of the formula XII: T=H.; X=H; P=—CO—O—C₄H₉; Y=3-pyridyl

A mixture of 1.5 g (5.3 mmol) of 4-amino-N-butoxycarbonyl-diphenylamine, 55 ml of pyridine and 0.94 g (5.3 mmol) of 3-pyridylsulfonyt chloride is stirred for 1 hour at OaC and then poured into a mixture of ice+HCl.

The precipitate formed is spin-filtered off, rinsed with water and dried to give 2.06 g of a purple-coloured solid (yield=91.5%).

M.p.=170-171° C.

NMR (CDCl₃) δ (ppm):1.5(9 H, s); 7.1 (2 H, d, J=8.7Hz); 7.15-7.6 (8 H, m); 7.65 (1 H, s, exchangeable with CF₃COOD); 8.1 (1 H, d, J=8 Hz); 8.8 (1 H, d, J=4.5 Hz); 9.1 (1 H, s)

Step b:

Compound of the formula II: T=H; A=SO₂; X=H; Y=3-pyridyl

This compound is obtained by performing the process as in Example 1, step b) starting with the compound obtained in the above step a) (yield=46%).

M.p.=155-158° C.

NMR (acetone-d₆) δ (ppm): 7.0 (1H, m); 7.2 (6H, m); 7.35 (2H, m); 7.65 (1H, broad s); 7.7 (1H, m); 8.2 (1H, m); 8.9 (1H, m); 9.05 (2H, 2 s).

Step c:

Compound of the formula I: T=H.; A=SO₂; X=H.; Y=3-pyridyl

This compound is obtained by performing the process as in Example 1, step c) starting with the compound obtained in the above step b) (yield=76%).

M.p.=139-141° C.

NMR (acetone-d₆) δ (ppm): 7.0 (2H, m); 7.2-7.5 (9H, m); 8.0 (1H, m); 8.7 (1H, m); 8.8 (1H, 2 d).

### EXAMPLE 7

Compound of the formula I: T=H; X=—SO₂—C₆H₅; A=—SO₂; Y=—C₆H₅

Step a:

Compound of the formula XIII: T=H; Y=—C₆H₅; P=—CO—O—C₄H₉

A mixture composed of 1.5 g (5.2 mmol) of 4-amino-N-butoxycarbonyldiphenylamine, 60 ml of dichloromethane and 0.9 g (5.1 mmol) of benzenesulfonyl chloride and 0.63 g (6.24 mmol) of triethylamine is stirred for 20 hours at 20° C. and then poured into a mixture of ice+HCl and extracted with dichloromethane.

After washing the organic phase with H₂O and drying over Na₂SO₄, it is concentrated under vacuum to give a residue which is reacted in the presence of 0.92 g (5.2 mmol) of benzenesulfonyl chloride, 0.63 g (6.24 mmol) of triethylamine and 60 ml of dichloromethane.

After stirring for 3 days at 20° C., a further 0.92 g (5.2 mmol) of benzenesulfonyl chloride and 0.63 g (6.24 mmol) of triethylamine are added.

The reaction medium is stirred for a further 16 hours at 20° C. and then poured into a mixture of ice +HCl and extracted with dichloromethane.

The organic phase is rinsed with water, dried over Na₂SO₄ and concentrated to give 1.63 g of a pink solid (yield=56%).

M.p.=158-160° C.

NMR (DMSO) δ (ppm): 1.35 (9 H, s); 6.9 (2 H, m); 7.15- 7.3 (5 H, m); 7.35 (2 H, m); 7.6 (4 H, m); 7.85 (6 H, m).

Step b:

Compound of the formula II: T=H; X=SO₂—C₆H₅; A=SO₂; Y=—C₆H₅

This compound is obtained by performing the process as in Example 1, step b) starting with the compound obtained in the above step a) (yield=97%).

M.p.=183-185° C.

NMR (CDCl₃) δ (ppm): 6.7 (2H, d, J=8.9 Hz); 6.8 (2H, d, J=8.9 Hz); 6.95 (1H, m); 7.1 (2H, d, J=7.5 Hz); 7.15-7.35 (3H, m); 7.4-7.55 (4H, m); 7.55-7.7 (2H, m); 7.9 (4H, m).

Step c:

Compound of the formula I. T=H; X=—SO₂—C₆H₅; A=—SO₂-; Y=—C₆H₅

This compound is obtained by performing the process as in Example 1, step c) starting with the compound obtained in the above step b) (yield=40% after recrystallization from a mixture of ethyl acetate and hexane).

M.p.=150-153° C.

NMR (CDCl₃) δ (ppm): 6.9-7.1 (4H, m); 7.3-7.6 (9H, m); 7.65 (2H, m); 7.9 (4H, m)

### EXAMPLE 8

Compound of the formula I: T=H; X=H; A=—CO—NH—; Y=—C₆H₅

Step a:

Compound of the form XIV: T=H; X=H; Y=C₆H₅; P=—CO—O—C₄H₉

1 g (4 mmol) of 4-amino-N-butoxycarbonyldiphenylamine dissolved in 10 ml of dichloromethane is added to a solution of 0.44 g (3.7 mmol) of phenyl isocyanate and 10 ml of dichloromethane.

After stirring for 4 hours at room temperature, the medium is poured into water and then extracted with dichloromethane.

The organic phase is washed with 1N HCl solution, rinsed with H₂O until neutral and then dried over Na₂SO₄.

After concentration under vacuum, 1.35 g of a beige-coloured solid are obtained (yield=91%).

NMR (CDCl₃) δ (ppm): 1.35 (9 H, s): 6.85-7.3 (16 H, m, of which 2 H exchangeable with CF₃COOD).

Step b:

Compound of the formula II: T=H; X=H; A=—CO—NH—; Y=—C₆H₅

This compound is obtained by performing the process as in Example 1, step b) starting with the compound obtained in the above step a) (yield=86%).

M.p.=197-202° C.

NMR (DMSO-d₆) δ (ppm): 6.7 (1H, m); 6.8-7.0 (5H, m); 7.05-7.3 (6H, m, of which IH exchangeable with CF₃COOD); 7.35 (2H, m); 8.5 (1H, s, exchangeable with CF₃COOD); 8.6 (1 H, s, exchangeable with CF₃COOD).

Stec c:

Compound of the formula I: T=H; X=H; A=—CO—NH—; Y=—C₆H₅

The compound is obtained by performing the process as in Example 1, step c) starting with the compound obtained in the above step b) (yield=42%).

M.p.=151-152° C.

NMR (CDCl₃) δ (ppm): 6.8 (1H, m); 6.8-7.1 (4H, m); 7.2-7.5 (11 H, m).

### EXAMPLE 9

Compound of the formula I: T=H.; X=H; Y=—C₆H₅; A=—CO—NH—SO₂—

Step a:

Compound of the formula XVI: T=H; X=H; Y=—C₆H₅; P=—CO—O—C₄H₉

1.28 g (7 mmol) of benzenesulfonyl isocyanate are added to a mixture of 2 g (7 mmol) of 4-amino-N-butoxycarbonyldiphenylamine and 0.85 g (8.4 mmol) of triethylamine in 60 ml of dichloromethane.

The reaction medium is stirred for 20 hours at room temperature and then poured into a mixture of ice +HCl and extracted with dichloromethane.

The organic phase is washed with H₂O, dried over Na₂SO₄ and concentrated under vacuum.

The pasty residue obtained is triturated in heptane to give 3.29 g of crystals which are filtered off and dried (quantitative yield).

M.p.=169-172° C.

NMR (CDCl₃) δ (ppm): 1.4 (9 H, s); 6.9-7.3 (9 H, m, of which 1 H exchangeable with CF₃COOD); 7.4-7.6 (3 H, m); 7.8 (2 H, m); 8.1 (1 H, broad s, exchangeable with CF₃COOD).

Step b:

Compound of the formula II: T=H; X=H; Y=—C₆H₅; A=—CO—NH—SO₂—

This compound is obtained by performing the process as in Example 1, step b) starting with the compound obtained in the above step a) (yield=94%).

M.p.=78° C.

NMR (CDCl₃) δ (ppm): 6.8 (1H, m); 6.9 (4H, m); 7.1-7.2 (5H, m) 7.4-7.5 (2H, m); 7.5-7.6 (1H, m); 7.8 (2H, m); 8.2 (1H, broad s).

Step c:

Compound of the formula I: T=H.; X=H; Y=—C₆H₅; A=—CO—NH—SO₂—

This compound is obtained by performing the process as in Example 1, step c) starting with the compound obtained in the above step b) (yield=71%).

M.p.=102-104° C.

NMR (CDCl₃) δ (ppm): 6.9 (2H, m); 7.2-7.7 (11H, m); 7.8 (2H, m); 8.5 and 8.6 (1H. 2 s).

### EXAMPLE 10

Compound of the formula I: T=4-OCH₃ ; X=—CH₃; A=CO; Y=3-pyridyl

Step a:

Compound of the forrnula X: T=4-OCH₃; P=—CO—O—C₄H₉

0.15 g (3.18 mmol) of formic acid is added to a solution of 1 g (3.18 mmol) of 4′-amino4-methoxy-N-butoxycarbonyidiphenylamine in 30 ml of toluene, and the mixture is stirred for 16 hours at room temperature and then refluxed while removing the water formed using Dean-Stark apparatus.

After refluxing for 3 hours, 0.75 g (15.9 mmol) of formic acid is added and heating is continued for 7 hours.

After cooling, the reaction medium is concentrated under vacuum.

The residue is purified by flash chromatography on silica in heptane/ethyl acetate (1:1) to give 0.74 g of a beige-coloured solid (yield=68%).

M.p.=131-138° C.

NMR (CDCl₃) δ (ppm): 1.4 (9 H, s); 3.7 (3 H, 2 s); 6.75 (2 H, m); 6.9 (2 H, d, J=8.75 Hz); 7.0-7.15 (3 H, m); 7.3 (2 H, m); 8.2 (1 H, s); 8.5 (1 H, 2 s).

Step b:

Compound of the formula III: T=4-OCH₃; X=CH₃; P=CO—O—C₄H₉

0.41 g (5.4 mmol) of dimethyl sulfide/borane is added to a solution maintained at 0° C., composed of 0.74 g (2.16 mmol) of the compound prepared in step a) above in 20 ml of dimethoxyethane.

After addition, the medium is maintained at 65° C. for 3 hours and then cooled to 0° C. 20 ml of water are then added and the mixture is extracted with dichloromethane.

The organic phase is washed with 10% NaOH solution and then rinsed with H₂O until neutral, and then dried over Na₂SO₄.

After concentration under vacuum, 0.73 g of a beige-coloured solid is obtained (quantitative yield).

M.p.=130-134° C.

NMR (CDCl₃) δ (ppm): 1.35 (9 H, s); 2.75 (3 H, s); 3.7 (4 H, s, of which 1 H exchangeable with D₂O); 6.45 (2 H, m); 6.7 (2 H, m); 6.9 (2 H, m); 7.1 (2 H, m).

Step c:

Compound of the formula IV: T=4-OCH₃; X=—CH₃; Y=3-pyridyl; P=—CO—O— C₄H₉

This compound is obtained by performing the process as in Example 1, step a) starting with the compound obtained in the above step b) (yield=69%).

Oil

NMR (CDCl₃) δ (ppm): 1.3 (9H, s); 3.4 (3H, s); 3.7 (3H, s); 6.8 (2H, m); 6.9 (2H, m); 6.95-7.1 (5H, m); 7.5 (1H, m); 8.4 (1H, m); 8.45 (1H, m).

Step d:

Compound of the formula II: T=4-OCH₃; X=CH₃; Y=3-pyridyl; A=CO

This compound is obtained by performing the process as in Example 1, step b) starting with the compound obtained in the above step c) (yield=82%).

M.p.=100-106° C.

NMR (DMSO-d₆) δ (ppm): 3.1 (3H, s); 3.45 (3H, s); 6.5 (2H, m); 6.65 (2H, m); 6.8 (4H, m); 7.15 (1H, m); 7.5 (IH, m); 7.8 (1H, broad s, exchangeable with CF₃COOD); 8.3 (2H, m).

Step e:

Compound of the formula I: T=4-OCH₃; X=—CH₃; A=CO; Y=3-pyridyl

This compound is obtained by performing the process as in Example 1, step c) starting with the compound obtained in the above step d) (yield=13%).

Oil

NMR (CDCl₃) δ (ppm); 3.4 (3H, s); 3.8 (3H, s); 6.8-7,3 (9H, m) 7.7 (1H, m); 8.5 (2H, m).

### EXAMPLE 11

Compound of the formula I: T=4-OCH₃; X=H; A=CO and Y=phenyl

Step a

Corresponding compound of the formula XXVIII

A solution of 10.06 g (32 mmol) of 4-amino4′-methoxy-N-butoxycarbonyidiphenylamine in 170 ml of a dichloroethane/acetic acid mixture (95:5) is added to a mixture of 21.33 g (16 mmol) of Argonaut Argopore MB—CHO resin (0.75 mmol/g) in 130 ml of a mixture of dichloroethane/acetic acid (95:5), followed by addition of a solution of 13.56 g (64 mmol) of sodium triacetoxyborohydride in 240 ml of a dichloroethane/acetic acid mixture (95:5). After stirring for 3 hours at room temperature, the resin is filtered off, washed successively with dichloromethane, DMF, methanol and then dichloromethane, and dried under vacuum to constant weight (25.38 g).

Step b

Corresponding compound of the formula II

110 μl (1 mmol) of N-methylmorpholine and 58 μl (0.5 mmol) of benzoyl chloride are added to a mixture of 133 mg (0.1 mmol) of the resin prepared in Example 11a) and 1.2 ml of dichloromethane. After stirring for 3½ hours at room temperature, the resin is filtered off and washed with a dichloromethane/dichloroethane mixture (4:1), with DMF, methanol and with a dichloromethane/dichloroethane mixture (4:1).

The resin is then taken up in 2 ml of a dichloromethane/trifluoro-acetic acid mixture (7:3) and stirred at room temperature for 6 hours, and then filtered off and rinsed with dichioromethane. The filtrate is concentrated under vacuum to give 18.1 mg of product (yield=57%).

NMR (DMSO-d₆) δ (ppm) 3.8 (3H, s); 6.8-7.1 (6H, m); 7.5 (6H, m, of which 1H exchangeable with CF₃COOD); 7.9 (2H,m); 10.0 (1H, s, exchangeable with CF₃COOD).

Step c

Title Compound

This compound is obtained by performing the process as in Example 4, starting with the compound obtained in the above step b).

### EXAMPLE 45

Compound of the formula I: T=H; X=H; A=SO₂ and Y=phenyl

Step a

Corresponding compound of the formula XXVIII

This resin is obtained by performing the process as in Example 11a), starting with 4-amino-N-butoxycarbonyidiphenylamine.

Step b

Corresponding compound of the formula II

2.47 ml (22.5 mmol) of N-methylmorpholine are added to a suspension of 3.55 g (equivalent to 2.248 mmol) of the resin prepared in Example 11a) in 40 ml of dichloromethane, followed by addition of 1.434 ml (11.25 mmol) of benzenesulfonyl chloride. After stirring for 3 hours at room temperature, the resin is filtered off, washed successively with dichloromethane, DMF, methanol and dichloromethane, and dried under vacuum.

The resin is then resuspended in 40 ml of a dichloromethane/trffluoroacetic acid mixture (95:5) containing 40 μl of water. After stirring for 2 hours at room temperature, the resin is filtered off and rinsed with dichloromethane. The filtrate is concentrated under vacuum to give an amorphous solid (yield=73%).

NMR (CDCl₃) δ (ppm)=4.2 (2H, broad s, exchangeable with CF₃COOD); 6.9-7.1 (7H, m); 7.2 (2H, m); 7.3-7.6 (3H, m); 7.7 (2H, m).

Step c

Title Compound

This compound is obtained by performing the process as in Example 4, starting with the compound obtained in the above step b).

### EXAMPLE 66

Compound of the formula I: T=H; X=H.; A=CO; Y=2,4-dimethoxyphenyl.

Step a

Compound of the formula IV: T=H; X=H; A=CO; Y=2,4-dimethoxyphenyl

A mixture of 94.7 mg (0.33 mmol) of 4-amino-N-butoxycarbonyidiphenylamine, 90.2 mg (0.45 mmol) of 2,4-dimethoxybenzoyl chloride, 163 mg of N,N-(diisopropyl)aminomethylpolystyrene resin (3.68 mmol/g) and 3 ml of 1,2dichloroethane is stirred for 11 hours at room temperature.

The excess aniline derivative is trapped by adding 136 mg of polystyrene methyl isocyanate resin with stirring for 14 hours at room temperature, followed by 3 hours at 60° C.

The excess acid chloride is removed by reaction with 200 mg of polystyrene aminomethyl resin with stirring at room temperature for 4 hours.

The resins are then filtered off and rinsed with CH₂Cl₂. The filtrate, concentrated under vacuum, gives 85 mg of a beige-coloured solid (yield=57%).

NMR (CDCl₃) δ (ppm)=1.3 (9H, s); 3.8 (3H, s); 3.95 (3H, s); 6.45 (1H, d, J=2.2 Hz); 6.55 (1H, dd, J=2.2 Hz and 8.8 Hz); 7.0-7.3 (7H, m); 7.5 (2H, m); 8.15 (1H, d, J=8.8 Hz); 9.6 (1H, broad s).

Step b

Corresponding compound of the formula II

This compound is obtained by carrying out a process similar to that illustrated in Example 1b).

(Yield=99%).

NMR (CDCl₃) δ (ppm)=3.8 (3H, s); 3.95 (3H, s); 6.4 (1H, d, J=2.2 Hz); 6.6 (1H, dd, J=2.2 Hz and 8.8 Hz); 6.7 (1H, broad s); 6.8 (1H, m); 7.0 (3H, m); 7.2 (2H, m); 7.4 (2H, d, J=8.8 Hz); 8.2 (2H, d, J=8.8 Hz); 9.65 (1 H. broad s).

Step c

Title Compound

This compound is obtained by carrying out a process similar to that illustrated in Example 4.

(Yield=66%).

### EXAMPLE 121

Compound of the formula I: T=H.; X=H; Y=3-methyl-5chloro-2-benzothienyl; A=SO₂

Step a

Compound of the formula XII: T=X; X=H ; A=SO₂; P=t-butoxycarbonyl; Y=3-methyl-5chloro-2-benzothienyl

A mixture of 99.5 mg (0.35 mmol) of 4-amino-N-butoxycarbonyl-diphenylamine, 147.6 mg (0.525 mmol) of 5-chloro-3-methylbenzo[b]thiophene-2-sulfonyl chloride and 192 mg (3.64 mmol/g) of morpholinomethylpolystyrene resin in 6 ml of CH₂Cl₂ is stirred for 4 hours at room temperature.

760 mg (1.39 mmol/g) of polystyrene aminomethyl resin and 440 mg (1.19 mmol/g) of polystyrene methyl isocyanate resin are then added. After stirring for 10 hours at room temperature, the resins are filtered off. 380 mg (1.39 mmol/g) of polystyrene aminomethyl resin and 440 mg (1.19 mmol/g) of polystyrene methyl isocyanate resin are added to the filtrate and the mixture is stirred at room temperature for 10 hours, followed by addition of 440 mg of polystyrene methyl isocyanate resin and stirring for a further 10 hours. The resins are then filtered off and the filtrate is concentrated under vacuum to give 82 mg of cream-coloured crystals (yield=44%).

NMR (DMSO-d₆) δ (ppm)=1.3 (9H, s); 2.4 (3H, s); 7.0-7.2 (7H, m) 7.25 (2H, m);7.5 (1H, dd, J=2Hz and 8.7Hz); 7.9 (1H, d, J=2 Hz); 8.0 (1H, d, J=8.7 Hz); 10.7 (1H, s).

Step b

Corresponding compound of the formula II

This compound is obtained by carrying out a process similar to that illustrated in Example 1b.

NMR (acetone-d₆): 2.3 (3H, s); 6.8 (1H, m); 6.8-7.0 (6H, m); 7.1 (2H, t, J=7.3 Hz); 7.5 (1H, dd, J=2 and 8.7 Hz); 7.8 (1H, d, J=1.8 Hz); 7.9 (1H, d, J=8.7 Hz); 7.9 (1 H, s).

Step c

This compound is obtained by carrying out a process similar to that illustrated in Example 4.

The compounds of Examples 12 to 44, 46 to 65, 67 to 120 and 122 to 251 in Tables 1 to 7 below are prepared in a similar manner using one of the procedures illustrated in Examples 1 to 121 above.

The compounds of Table 1 correspond to the following formula:

TABLE 1

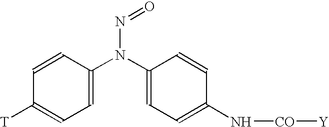

| Ex- | | | |
|---|---|---|---|
| am- | | | |
| ple | | | |
| No. | T | Y | NMR |
| 11 | —OCH₃ | —C₆H₅ | (DMSO-d6): 3.7(3H, 2s); |
| | | | 7.0(3H, m); |
| | | | 7.35(2H, t, J=9.2Hz); |
| | | | 7.4(4H, m); 7.9(4H, m); |
| | | | 10.4(1H, 2s, |
| | | | exchangeable with |
| | | | CF3COOD) |
| 12 | —OCH₃ | 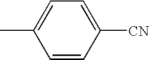 | (Acetone-d6): 3.9(3H, 2s); 6.9-7.3(4H, m); 7.4-7.6(2H, m); 7.9-8.15(4H, m); 8.15-8.3(2H, m); 9.99(1H, 2s broad, exchangeable) |
| 13 | —OCH₃ | 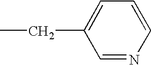 | (DMSO-d6): 3.79(3H, 2s); 5.75(2H, s); 6.90-7.30(3H, m); 7.30-7.70(4H, m); 7.70-7.85(3H, m); 8.40-8.60(2H, m); 10.46(1H, 2s broad) |
| 14 | —CF₃ | 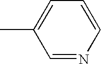 | (CDCl3): 7.8(1H, d, J=8.67Hz); 7.25(1H, m); 7.30-8.00(7H, m); 8.00-8.40(2H, m); 8.80(1H, m); 9.11(1H, m) |
| 15 | —OCH₃ | 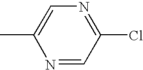 | (Acetone-d6): 3.90(3H, 2s); 7.00-7.70(6H, m); 8.00-8.25(2H, m); 8.85(1H, m); 9.20(1H, m); 10.36(1H, 2s broad) |
| 16 | —OCH₃ | 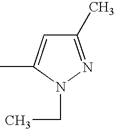 | (DMSO-d6): 1.17(3H, t, J=7.13Hz); 2.21(3H, s); 3.80(3H, 2s); 4.42(2H, q, J=7.13Hz); 6.84(1H, s); 7.05-7.20(4H, m); 7.37(2H, m); 7.87(2H, m); 10.32(1H, 2s broad) |
| 17 | —OCH₃ | 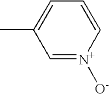 | (DMSO-d6): 3.80(3H, 2s); 7.00-7.25(2H, m); 7.30-7.70(4H, m); 7.70-8.00(4H, m); 8.41(1H, m); 8.75(1H, m); 10.66(1H, 2s broad, exchanged with D₂O) |
| 18 | —OCH₃ | 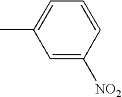 | (Acetone-d6): 3.90(3H, 2s); 7.02-7.28(4H, m); 7.36-7.55(2H, m); 7.90(1H, m); 7.95-8.12(2H, m); 8.49(2H, m); 8.88(1H, m); 10.15(1H, 2s broad) |
| 19 | —OCH₃ | 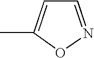 | (Acetone-d6): 3.90(3H, 2s); 7.00-7.30(5H, m); 7.30-7.55(2H, m); 7.90-8.15(2H, m); 8.66(1H, m); 10.09(1H, 2s) |
| 20 | —SCH₃ | —C₆H₅ | (DMSO-d6): 3.32(3H, s); |
| | | | 7.14(2H, m); |
| | | | 7.30-7.50(4H, m); |
| | | | 7.5 to 7.7(3H, m); |
| | | | 7.80-8.10(4H, m); |
| | | | 10.47(1H, 2s broad) |
| 21 | —OCH₃ | 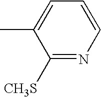 | (DMSO-d6): 2.48(3H, m); 3.80(3H, 2s); 6.85-7.50(7H, m); 7.65-8.10(3H, m); 8.60(1H, m); 10.67(1H, 2s broad) |
| 22 | —OCH₃ | 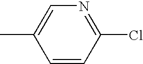 | (Acetone-d6): 3.90(3H, 2s); 7.00-7.25(4H, m); 7.35-7.55 (2H, m); 7.45(1H, m); 7.85-8.15(2H, m); 8.43(1H, m); 9.04(1H, d, J=2.41Hz); 9.01(1H, 2 broad s, exchangeable). |
| 23 | —OCH₃ | 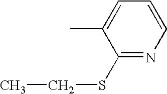 | (Acetone-d6): 1.23(3H, t, J=7.34Hz); 3.11(2H, q, J=7.34Hz); 3.78(3H, 2s); 6.80-7.45(7H, m); 7.70-8.00(3H, m); 8.47(1H, m); 9.70(1H, 2s broad) |
| 24 | —OCH₃ | 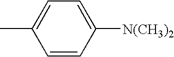 | (Acetone-d6): 3.09(6H, s); 3.90(3H, 2s); 6.82(2H, m); 7.13(4H, m); 7.42(2H, m); 7.75-8.15(4H, m); 9.45(1H, 2s broad) |

The compounds of Table 2 correspond to the formula:

TABLE 2

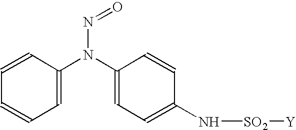

| Example No. | Y | NMR |
|---|---|---|
| 25 | 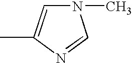 | (Acetone-d6): 3.90(3H, 2s); 7.07(2H, d, J=8.86Hz); 7.17(1H, dd, J=8.33Hz, J=1.51Hz); 7.34(1H, d, J=9.08Hz); 7.38-7.47(2H, m); 7.47-7.63(3H, m); 7.63-7.71(1H, m); 7.77(1H, dd, J=1.23Hz; J=17.14Hz) |
| 26 | 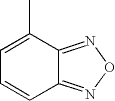 | (Acetone-d6): 6.75-7.80(10H, m); 7.80-8.35(2H, m); 9.70(1H, 2s broad) |
| 27 | 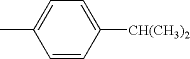 | (Acetone-d6): 1.25(3H, m); 1.30(3H, m); 3.04(1H, m); 6.90-7.30(3H, m); 7.30-7.70(8H, m); 7.70-7.95(2H, m); 9.28(1H, 2s broad) |
| 28 | 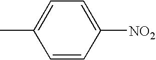 | (Acetone-d6): 7.00-7.35(2H, m); 7.35-7.75(7H, m); 8.00-8.30(2H, m); 8.30-8.65(2H, m); 9.59(1H, 2s broad) |
| 29 | 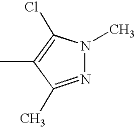 | (Acetone-d6): 2.33(3H, 2s); 3.80(3H, 2s); 6.95-7.30(2H, m); 7.30-8.15(7H, m); 9.45(1H, 2s) |
| 30 | 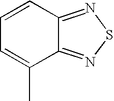 | (Acetone-d6): 6.95-7.15(2H, m); 7.15-7.75(7H, m); 7.75-8.20(2H, m); 8.20-8.60(2H, m); 9.59(1H, 2s broad) |
| 31 | 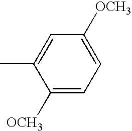 | (Acetone-d6): 3.82(3H, 2s); 3.97(3H, 2s); 7.00-7.25(3H, m); 7.25 to 7.70(9H, m); 9.61(1H, 2s broad) |
| 32 | 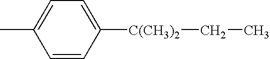 | (Acetone-d6): 0.65(3H, m); 1.32(6H, 2s); 1.70(2H, m); 6.80-7.30(3H, m); 7.30-8.25(10H, m); 9.26(1H, 2s broad) |
| 33 | 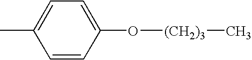 | (Acetone-d6): 0.99(3H, m); 1.52(2H, m); 1.79(2H, m); 4.07(2H, m); 6.70-8.25(13H, m); 9.19(1H, 2s broad) |
| 34 | 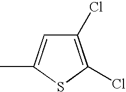 | (Acetone-d6): 6.80-7.30(2H, m); 7.30-8.25(8H, m); 9.71(1H, 2s broad) |
| 35 | 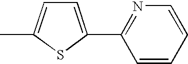 | (Acetone-d6): 7.00-7.80(12H, m); 7.80-8.15(2H, m); 8.60(1H, m); 9.50(1H, 2s broad) |
| 36 | 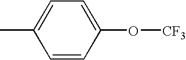 | (Acetone-d6): 6.85-7.30(4H, m); 7.30-7.75(7H, m); 7.80-8.15(2H, m); 9.43(1H, 2s exchangeable) |
| 37 | 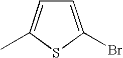 | (Acetone-d6): 6.85-7.35(4H, m); 7.35-8.20(7H, m); 9.53 (1H, 2s broad) |
| 38 | 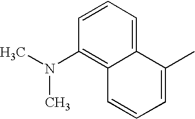 | (Acetone-d6): 2.90(6H, s); 6.45-7.80(13H, m); 7.80-8.75(2H, m); 9.64(1H, 2s) |
| 39 | 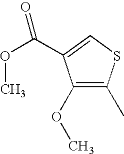 | (Acetone-d6): 3.88(3H, 2s); 3.99(3H, 2s); 6.65-7.85(9H, m); 8.5(1H, m); 9.66(1H, 2s) |
| 40 | 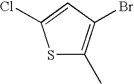 | (Acetone-d6): 6.30-8.15(9H, m); 8.45(1H, m); 9.97(1H, 2s broad) |
| 41 | 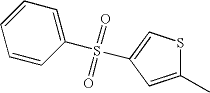 | (Acetone-d6): 6.90-7.30(2H, m); 7.30-7.50(4H, m); 7.50-7.85(7H, m); 7.90-8.15(2H, m); 8.66(1H, m); 9.57(1H, 2s broad) |
| 42 | 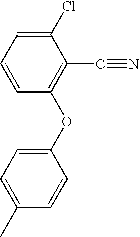 | (Acetone-d6): 7.00-7.30(3H, m); 7.30-7.70(9H, m); 7.7-7.85(1H, m); 7.85-8.25(3H, m); 9.34(1H, 2s) |
| 43 | 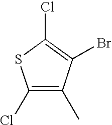 | (Acetone-d6): 6.75-7.85(8H, m); 8.45(1H, m); 9.93(1H, 2s broad) |
| 44 | —CH₃ | (Acetone-d6): 7.05-7.40(3H, m); |
| | | 7.40-7.80(9H, m) |
| 45 | 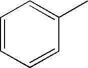 | (CDCl3): 6.75-7.00(2H, m); 7.00-7.40(10H, m); 7.40-7.95(2H, m) |
| 46 | 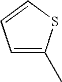 | (Acetone-d6): 6.75-7.75(10H, m); 7.75-8.25(2H, m); 9.30(1H, 2s broad) |
| 47 | 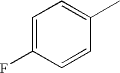 | (Acetone-d6): 6.85-7.30(4H, m); 7.30-7.65(7H, m); 6.65-8.10(2H, m); 9.34(1H, 2s broad) |
| 48 | 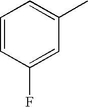 | (Acetone-d6): 6.90-7.35(3H, m); 7.35-8.10(10H, m); 9.41(1H, 2s broad) |
| 49 | 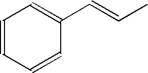 | (Acetone-d6): 6.75-7.35(4H, m); 7.35-8.30(12H, m); 9.13(1H, 2s broad) |
| 50 | 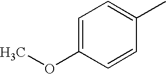 | (Acetone-d6): 3.90(3H, 2s); 6.95-7.30(5H, m); 7.30-7.65(6H, m); 7.65-7.90(2H, m); 9.20(1H, 2s broad) |
| 51 | 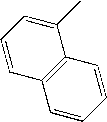 | (Acetone-d6): 6.75-7.85(12H, m); 7.90-9.00(4H, m); 9.70(1H, 2s broad) |
| 52 | 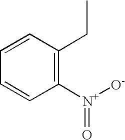 | (Acetone-d6): 4.02(2H, m); 6.40-7.30(2H, m); 7.30-7.90(9H, m); 7.90-8.20(1H, m) |
| 53 | 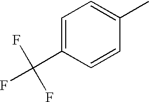 | (Acetone-d6): 6.65-7.35(3H, m); 7.35-7.90(9H, m); 8.10-8.45(1H, m); 9.15(1H, 2s broad) |
| 54 | 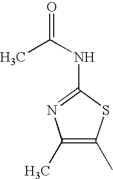 | (Acetone-d6): 2.88(6H, s); 6.95-7.35(2H, m); 7.35-7.65(4H, m); 7.85-8.25(3H, m); 9.53(1H, 2s broad) |
| 55 | 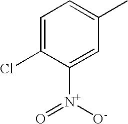 | (Acetone-d6): 6.95-7.35(4H, m); 7.35-7.85(8H, m); 11.28(1H, 2s broad) |

The compounds of Table 3 correspond to the formula:

TABLE 3

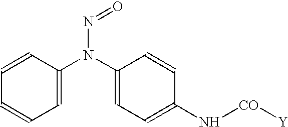

| Ex- | | |
|---|---|---|
| am- | | |
| ple | | |
| No. | Y | NMR |
| 56 | 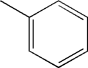 | (CDCl3): 7.1(2H, d, J=6.9Hz); 7.2-7.7(8H, m); 7.7-8.0(5H, m) |
| 57 | 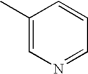 | (CDCl3): 7.05(2H, m); 7.2-7.5(6H, m); 7.65(1H, d, J=8.9Hz); 7.75(1H, d, J=8.9Hz); 8.0-8.1(1H, 2s); 8.15(1H, d, J=8Hz); 8.7(1H, broad s); 9.0(1H, broad s) |
| 58 | 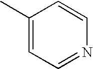 | (DMSO-d6): 7.0(2H, d, J=8.7Hz); 7.3(2H, d, J=7.5Hz); 7.4(3H, m); 7.8(4H, m); 8.7(2H, m); 10.5(1H, 2s) |
| 59 | 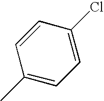 | Mass spectrum: After loss of No. M + 1 = 323.79 (detected) |
| 60 | 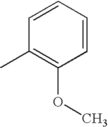 | Mass spectrum: After loss of No. M + 1 = 319.1 (detected) |
| 61 | 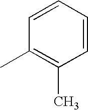 | (CDCl3): 2.40(3H, broad s); 7.00-7.20(2H, m); 7.20-7.60(9H, m); 7.60-7.90(2H, m) |
| 62 | 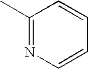 | (CDCl3): 6.92(2H, m); 7.14-7.42(6H, m); 7.60-7.85(3H, m); 8.11(1H, d, J=7.83Hz); 8.44(1H, d, J=4.65Hz); 9.97(1H, 2s broad, exchanged with TFA) |
| 63 | 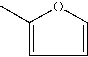 | (CDCl3): 6.58(1H, m); 7.09(2H, d, J=6.70Hz); 7.22-7.57(7H, m); 7.71(1H, d, J=9.02Hz); 7.80Hz(1H, d, J=8.83Hz); 8.16(1H, 2s broad) |
| 64 | 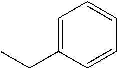 | (CDCl3): 3.63(2H, s); 6.89(2H, m); 7.00-7.40(11H, m); 7.43(1H, d, J=8.64Hz) |
| 65 | 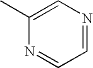 | (CDCl3): 7.10(2H, m); 7.27-7.57(5H, m); 7.82(1H, d, J=8.97Hz); 7.91(1H, d, J=8.80Hz); 8.59(1H, m); 8.82(1H, dd, J=2.55Hz, J=3.7Hz); 9.51(1H, t, J=1.63Hz); 9.78(1H, 2s broad) |
| 66 | 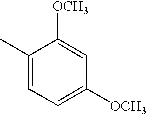 | (CDCl3): 3.70(3H, 2s): 3.85(3H, 2s); 6.34(1H, m); 6.48(1H, m); 6.90(2H, m); 7.10-7.38(5H, m); 7.55(1H, d, J=8.99Hz); 7.62(1H, d, J=8.82Hz); 8.06(1H, d, J=8.86Hz); 9.63(1H, 2s broad) |
| 67 | 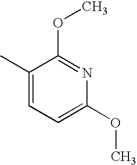 | (CDCl3): 3.82(3H, 2s); 4.01(3H, 2s); 6.32(1H, dd, J=2.2Hz, J=8.43Hz); 6.90(2H, m); 7.03-7.42(5H, m); 7.53(1H, dd, J=2.2Hz, J=9.04Hz); 7.62(1H, dd, J=2.2Hz, J=8.79Hz); 8.28(1H, dd, J=2.51Hz, 8.33Hz); 9.58(1H, 2s broad) |
| 68 | 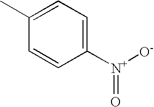 | (Acetone-d6): 7.22(2H, d, J=8.86Hz); 7.40-7.70(5H, m); 8.02(1H, d, J=12.0Hz); 8.09(1H, d, J=8.88Hz); 8.31(2H, d, J=9.0Hz); 8.42(2H, dd, J=2.26Hz, J=11.24Hz) |
| 69 | 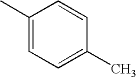 | (CDCl3): 2.43(3H, s); 7.08(2H, m); 7.20-7.60(7H, m); 7.65-8.00(4H, m) |
| 70 | 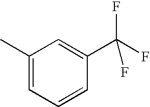 | (CDCl3): 7.07(2H, m); 7.40-7.55(4H, m); 7.60-7.75(2H, m); 7.75-7.90(2H, m); 7.90-8.20(3H, m) |
| 71 | 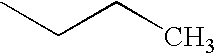 | (CDCl3): 1.01(3H, t, J=7.30Hz); 1.76(2H, m); 2.35(2H, m); 7.07(1H, m); 7.15-7.60(7H, m); 7.66(1H, m) |
| 72 | 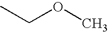 | (CDCl3): 3.51(3H, s); 4.03(2H, s); 7.06(2H, m); 7.25-7.55(5H, m); 1.63(1H, d, J=8.96Hz); 7.72(1H, d, J=8.77Hz); 8.35(1H, 2s broad) |
| 73 | 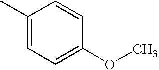 | (CDCl3): 3.87(3H, s); 6.98(2H, m); 7.07(2H, m); 7.30-7.60(4H, m); 7.60-7.95(5H, m) |
| 74 | 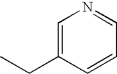 | (DMSO-d6): 3.73(2H, 2s); 7.14(2H, m); 7.30-7.45(4H, m); 7.45-7.60(2H, m); 7.65-7.85(3H, m); 8.40-8.60(2H, m); 10.47(1H, 2s broad, exchanged with TFA) |
| 75 | 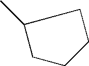 | (DMSO-d6): 1.40-1.95(8H, m); 2.78(1H, m); 7.09(1H, d, J=9.04Hz); 7.16(1H, m); 7.29-7.43(3H, m); 7.43-7.61(2H, m); 7.73(1H, d, J=9.04Hz); 7.77(1H, d, J=8.67Hz); 10.09(1H, 2s, broad, exchanged with D₂O) |
| 76 | 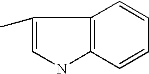 | (DMSO-d6): 7.05-7.30(3H, m); 7.30-7.70(7H, m); 8.15-8.45(3H, m); 8.81(1H, m); 10.39(1H, s) |

The compounds of Table 4 correspond to the formula:

TABLE 4

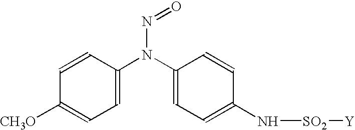

| Example | | |
|---|---|---|
| No. | Y | NMR |
| 77 | —CH₃ | (Acetone-d6): 3.10(3H, 2s); |
| | | 3.89(3H, 2s); |
| | | 6.95-7.20(4H, m); |
| | | 7.20-7.60(4H, m); |
| | | 8.85(1H, 2s broad) |
| 78 | 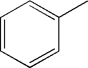 | (Acetone-d6): 3.89(3H, 2s); 6.95-7.20(4H, m); 7.25-7.45(4H, m); 7.55-7.75(3H, m); 7.80-7.95(2H, m); 9.30(1H, 2s broad) |
| 79 | 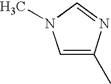 | (Acetone-d6): 3.78(3H, 2s); 3.89(3H, 2s); 6.95-7.20(4H, m); 7.20-7.55(4H, m); 7.55-7.80(2H, m); 9.20(1H, 2s broad) |
| 80 | 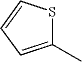 | (Acetone-d6): 3.89(3H, 2s); 7.00-7.30(5H, m); 7.30-7.55(4H, m); 7.55-7.75(1H, m); 7.75-7.95(1H, m); 9.40(1H, 2s broad) |
| 81 | 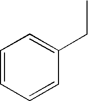 | (Acetone-d6): 2.42(2H, s); 3.89(3H, 2s); 6.95-7.20(5H, m); 7.20-7.45(7H, m); 7.60-7.85(2H, m); 9.23(1H, 2s broad) |
| 82 | 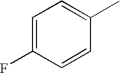 | (Acetone-d6): 3.89(3H, 2s); 6.80-7.20(4H, m); 7.20-7.85(8H, m); 9.38(1H, 2s broad) |
| 83 | 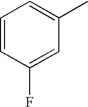 | (Acetone-d6): 3.89(3H, 2s); 6.95-7.20(4H, m); 7.20-7.55(6H, m); 7.75-8.10(2H, m); 9.32(1H, 2s broad) |
| 84 | 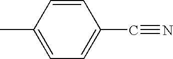 | (Acetone-d6): 3.89(3H, 2s); 6.95-7.20(4H, m); 7.20-7.50(4H, m); 7.85-8.15(4H, m); 9.52(1H, 2s broad) |
| 85 | 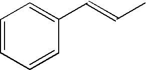 | (Acetone-d6): 3.89(3H, 2s); 7.00-7.17(4H, m); 7.17-7.32(1H, m); 7.31-7.60(8H, m); 7.65-7.80(2H, m); 9.11(1H, 2s broad |
| 86 | 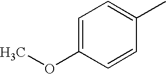 | (Acetone-d6): 3.89(6H, 4s); 6.92-7.25(6H, m); 7.25-7.50(4H, m); 7.72-7.90(2H, m); 9.17(1H, 2s broad) |
| 87 | 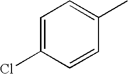 | (Acetone-d6): 3.89(3H, 2s); 6.95-7.20(4H, m); 7.20-7.40(4H, m); 7.55-7.75(2H, m); 7.75-7.95(2H, m); 9.35(1H, 2s broad) |
| 88 | 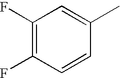 | (Acetone-d6): 3.89(3H, 2s); 6.85-7.20(4H, m); 7.20-7.50(4H, m); 7.50-7.70(1H, m); 7.70-8.10(2H, m); 9.39(1H, 2s broad) |
| 89 | 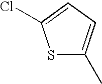 | (Acetone-d6): 3.89(3H, 2s); 7.00-7.25(5H, m); 7.25-7.60 (5H, m); 9.53(1H, 2s broad) |
| 90 | 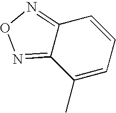 | (Acetone-d6): 3.89(3H, 2s); 6.90-7.20(4H, m); 7.20-7.60(4H, m); 7.70-7.90(1H, m); 8.20-8.40(2H, m); 9.81(1H, 2s broad) |
| 91 | 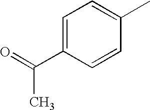 | (Acetone-d6): 2.66(3H, 2s); 3.89(3H, 2s); 6.95-7.20(4H, m); 7.30-7.50(4H, m); 7.90-8.10(2H, m); 8.10-8.25(2H, m); 9.45(1H, 2s broad) |
| 92 | 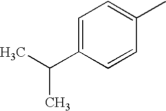 | (Acetone-d6): 1.28(6H, d, J=6.87Hz); 3.01(1H, m); 3.89(3H, 2s); 6.82-7.16(4H, m); 7.16-7.45(6H, m); 7.66-7.80(2H, m); 9.12(1H, 2s broad) |
| 93 | 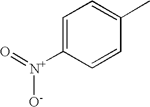 | (Acetone-d6): 3.89(3H, 2s); 6.40-7.70(8H, m); 7.85-8.70(4H, m); 9.57(1H, 2s broad) |
| 94 | 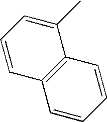 | (Acetone-d6): 3.89(3H, 2s); 6.90-7.20(4H, m); 7.20-7.45(3H, m); 7.55-7.90(3H, m); 7.95-8.50(4H, m); 8.70-8.95(1H, m); 9.65(1H, 2s broad) |
| 95 | 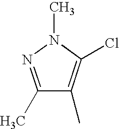 | (Acetone-d6): 2.30(3H, 2s); 3.81(3H, 2s); 3.89(3H, 2s); 6.90-7.20(4H, m); 7.20-7.60(4H, m); 9.41(1H, 2s broad) |
| 96 | 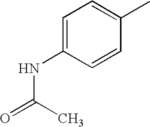 | (Acetone-d6): 3.81(3H, 2s); 3.89(3H, 2s); 6.80-7.20(6H, m); 7.20-7.52(3H, m); 7.52-8.00(3H, m) |
| 97 | 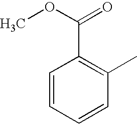 | (Acetone-d6): 3.89(3H, 2s); 4.03(3H, 2s); 6.95-7.20(4H, m); 7.20-7.50(4H, m); 7.60-8.10(4H, m); 8.73(1H, 2s broad) |
| 98 | 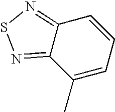 | (Acetone-d6): 3.89(3H, 2s); 6.95-7.20(4H, m); 7.20-7.55(4H, m); 7.80-8.05(1H, m); 8.30-8.60(2H, m); 9.58(1H, 2s broad) |
| 99 | 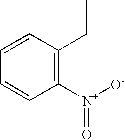 | (Acetone-d6): 3.89(3H, 2s); 8.16(2H, 2s); 7.05-7.20(4H, m); 7.30-7.50(7H, m); 8.80(1H, d, J=7.88Hz); 9.12(1H, 2s broad) |
| 100 | p-trifluoromethylphenyl | (Acetone-d6): 3.89(3H, 2s); |
| | | 6.95-7.20(4H, m); |
| | | 7.20-7.50(4H, m); |
| | | 7.85-8.25(4H, m); |
| | | 9.54(1H, 2s broad) |
| 101 | 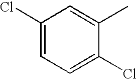 | (Acetone-d6): 3.89(3H, 2s); 6.80-7.20(6H, m); 7.25-7.55(3H, m); 7.75-8.10(2H, m); 9.73(1H, 2s broad) |
| 102 | 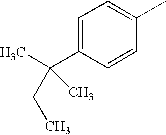 | (Acetone-d6): 0.64(3H, d, J=7.48Hz); 1.32Hz(6H, s); 1.70(2H, q, J=7.48Hz); 3.89(3H, 2s); 6.95-7.45(8H, m); 7.45-7.65(2H, m); 7.65-7.90(2H, m); 9.25(1H, 2s broad) |
| 103 | 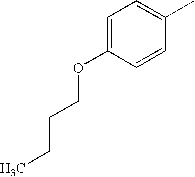 | (Acetone-d6): 0.99(3H, td, J=7.34Hz, J=2.23Hz); 1.51(2H, m); 1.79(2H, m); 3.89(3H, 2s); 4.09(2H, m); 7.00-7.25(6H, m); 7.30-7.50(4H, m); 7.70-7.95(2H, m); 9.16(1H, 2s broad) |
| 104 | 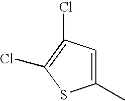 | (Acetone-d6): 3.89(3H, 2s); 7.00-7.25(4H, m); 7.30-7.65(5H, m); 9.65(1H, 2s broad) |
| 105 | 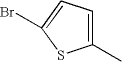 | (Acetone-d6): 3.89(3H, 2s); 7.00-7.20(4H, m); 7.20-7.35(1H, m); 7.35-7.55(5H, m); 9.52(1H, 2s broad) |
| 106 | 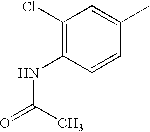 | (Acetone-d6): 2.28(3H, 2s); 3.89(3H, 2s); 6.95-7.20(4H, m); 7.20-7.45(4H, m); 7.60-7.90(3H, m); 8.30-8.65(1H, m); 9.35(1H, 2s broad) |
| 107 | 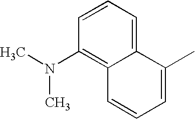 | (Acetone-d6): 2.89(6H, s); 3.89(3H, 2s); 6.80-7.20(5H, m); 7.20-7.50(4H, m); 7.50-7.80(2H, m); 8.25-8.75(3H, m); 9.71(1H, 2s broad) |
| 108 | 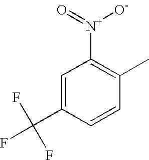 | (Acetone-d6): 3.89(3H, 2s); 6.90-7.30(4H, m); 7.30-7.60(3H, m); 7.95-8.55(4H, m); 9.65(1H, 2s broad) |
| 109 | 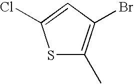 | (Acetone-d6): 3.89(3H, 2s); 7.00-7.25(4H, m); 7.25-7.60(5H, m); 9.93(1H, 2s broad) |
| 110 | 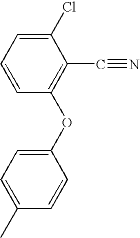 | (Acetone-d6): 3.89(3H, m); 6.95-7.20(5H, m); 7.20-7.50(6H, m); 7.50-7.65 (1H, d, J=8.19Hz); 7.65-7.85(1H, m); 7.85-8.10(2H, m); 9.32(1H, 2s broad) |
| 111 | 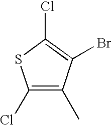 | (Acetone-d6): 3.89(3H, 2s); 6.80-7.30(5H, m); 7.30-7.65(3H, m); 9.91(1H, 2s broad) |
| 112 | 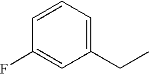 | (Acetone-d6): 3.89(3H, 2s); 4.62(2H, 2s); 6.90-7.35(8H, m); 7.35-7.65(4H, m); 9.97(1H, 2s broad) |
| 113 | 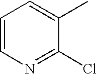 | (Acetone-d6): 3.89(3H, 2s); 6.95-7.55(8H, m); 7.70(1H, m); 8.40-8.80(2H, m); 9.91(1H, 2s broad) |

The compounds of Table 5 are compounds of the formula I in which X=H and A=SO₂. For Examples 114 to 120, T=4-OCH₃. For Examples 121 and 122, T=H.

TABLE 5

| Example | | |
|---|---|---|
| No. | Y | NMR |
| 114 | 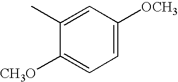 | (Acetone-d6): 3.70-4.10(9H, 3x2s); 7.00-7.25(6H, m); 7.25-7.55(5H, m); 9.10(1H, 2s broad) |
| 115 | 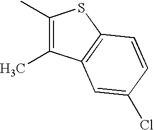 | (Acetone-d6): 2.63(3H, s); 3.89(3H, 2s); 6.95-7.20(4H, m); 7.20-7.55(4H, m); 7.59(1H, m); 7.90-8.20(2H, m); 9.70(1H, 2s broad) |
| 116 | 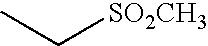 | (Acetone-d6): 1.33(3H, s); 3.31(2H, s); 3.89(3H, 2s); 6.95-7.30(4H, m); 7.30-7.70(4H, m); 9.28(1H, 2s broad) |
| 117 | 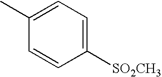 | (Acetone-d6): 1.33(3H, s); 3.89(3H, 2s); 7.00-7.20(4H, m); 7.20-7.50(4H, m); 8.05-8.15(4H, m); 9.31(1H, 2s broad) |
| 118 | 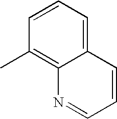 | (Acetone-d6): 3.89(3H, 2s); 6.90-7.15 (4H, m); 7.15-7.45 (4H, m); 7.70-7.90 (2H, m); 8.31(1H, dd, J=1.41Hz, J=8.27Hz); 8.43(1H, dd, J=1.41Hz, J=7.25Hz); 8.57(1H, dd, J=1.66Hz, J=8.37Hz); 9.2(1H, m); 9.35(1H, 2s broad) |
| 119 | 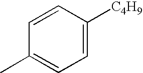 | (Acetone-d6): 0.93(3H, t, J=7.35Hz); 1.37(2H, m); 1.62(2H, m); 1.68(2H, m); 3.89(3H, 2s); 7.00-7.20(4H, m); 7.20-7.50(6H, m); 1.75-1.90(2H, m); 9.28(1H, 2s broad) |
| 120 | 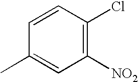 | (Acetone-d6): 3.89(3H, 2s); 6.95-7.20(4H, m); 7.20-7.50(4H, m); 7.99(1H, m); 8.09(1H, m); 8.35-8.55(1H, m); 9.60(1H, 2s broad) |
| 121 | 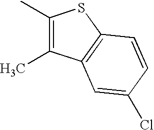 | (Acetone-d6): 2.68(3H, 2s); 7.00-7.25(3H, m); 7.25-7.70(7H, m); 7.90-8.20(2H, m); 9.72(1H, 2s broad) |
| 122 | 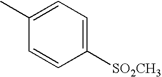 | (Acetone-d6): 3.24(3H, 2s); 7.00-7.35(3H, m); 7.35-7.70(6H, m); 7.95-8.30(4H, m); 9.59(1H, 2s broad) |

The compounds of Table 6 are compounds of the formula I in which T=4-OCH₃; X=H and A=CO.

TABLE 6

| Example | | |
|---|---|---|
| No. | Y | NMR |
| 123 | 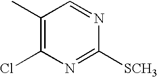 | (dmso-d6)=2.57(3H, 2s); 3.81(3H, 2s); 6.90-7.60(7H, m); 7.75-7.85(1H, m); 8.55-8.70(1H, m); 16.25(1H, 2s broad) |
| 124 | 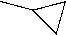 | (acetone-d6)=0.70-0.95(4H, m); 1.20-1.45(1H, m); 3.89(3H, 2s); 6.85-7.15(4H, m); 7.20-7.45(2H, m); 7.65-7.90(2H, m); 9.60(1H, 2s broad) |
| 125 | 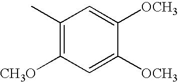 | (acetone-d6):=3.87(3H, s); 3.89(3H, 2s); 3.97(3H s); 4.17(3H, s); 6.80-7.25(5H, m); 7.25-7.55(2H, m); 7.55-7.80(1H, m); 7.80-8.10(2H, m); 10.16(1H, 2s broad) |
| 126 | 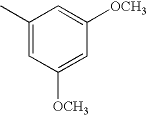 | (acetone-d6)=3.88(6H, s); 3.89(3H, 2s); 6.60-6.80(1H, m); 6.95-7.30(6H, m); 7.30-7.55(2H, m); 7.85-8.15(2H, m); 9.67(1H, 2s broad) |
| 127 | 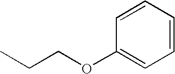 | (acetone-d6)=2.85-3.00(2H, m); 3.89(3H, 2s); 4.39(2H, m); 6.90-7.20(7H, m); 7.20-7.50(4H, m); 7.70-7.95(2H, m); 9.53(1H, 2s broad) |
| 128 | 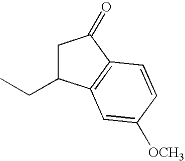 | (acetone-d6)=2.2-3.4(5H, m); 3.89(3H, 2s); 3.90(3H, 2s); 6.95-7.20(5H, m); 7.20-7.32(1H, m); 7.32-7.50(2H, m); 7.50-7.70(1H, m); 7.70-7.95(2H, m); 9.51(1H, 2s broad) |
| 129 | 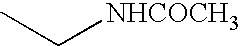 | (acetone-d6)=1.92(3H, 2s); 3.70-4.20(3H, 2s+2H, m); 6.65-7.90(7H, m); 7.90-8.10(1H, m); 8.90(1H, 2s broad) |
| 130 | 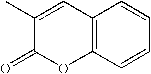 | (acetone-d6)=3.89(3H, 2s); 6.90-7.30(3H, m); 7.30-7.70(4H, m); 7.70-8.20(5H, m); 9.00-9.25(1H, m); 10.90(1H, 2s broad) |
| 131 | 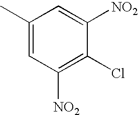 | (acetone-d6)=3.89(3H, 2s); 7.00-7.30(4H, m); 7.30-7.60(2H, m); 7.85-8.15(2H, m); 8.80-9.10(2H, m); 10.37(1H, 2s broad) |
| 132 | 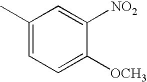 | (acetone-d6)=3.89(3H, 2s); 4.07(3H, s); 6.95-7.20(4H, m); 7.30-7.55(3H, m); 7.85-8.10(2H, m); 8.25-8.55(2H, m); 9.87(1H, 2s broad) |
| 133 | 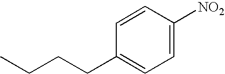 | (acetone-d6)=1.20-1.50(4H, m); 2.35-2.50(2H, m); 3.85(3H, 2s); 6.90-7.15(4H, m); 7.20-7.45(2H, m); 7.45-7.60(2H, m); 7.65-7.90(2H, m); 8.10-8.25(2H, m); 9.3(1H, 2s broad) |
| 134 | 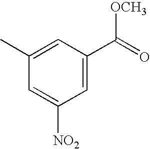 | (acetone-d6)=3.89(3H, 2s); 4.05(3H, s); 7.00-7.30(3H, m); 7.30-7.55(2H, m); 7.90-8.15(2H, m); 8.80-9.20(4H, m); 10.30(1H, 2s broad) |
| 135 | 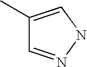 | (acetone-d6)=3.89(3H, 2s); 7.00-7.55(6H, m); 7.80-8.50(4H, m); 9.42(1H, 2s broad) |
| 136 | 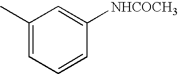 | (acetone-d6)=2.10(3H, s); 3.89(3H, 2s); 7.00-7.30(3H, m); 7.30-7.60(3H, m); 7.60-7.85(2H, m); 7.85-8.35(4H, m); 9.37(1H, broad s); 9.72(1H, 2s broad) |
| 137 | 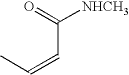 | (acetone-d6)=2.97(3H, s); 3.89(3H, 2s); 6.10-6.85(2H, m); 6.85-7.25(2H, m); 7.25-8.55(6H, m) |
| 138 | 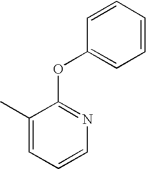 | (acetone-d6)=3.89(3H, 2s); 7.20-7.85(12H, m); 7.85-8.15(1H, m); 8.15-8.60(3H, m); 10.15(1H, 2s broad) |
| 139 | 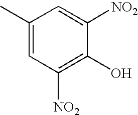 | (acetone-d6)=3.89(3H, 2s); 7.05-7.30(4H, m); 7.30-7.60(2H, m); 7.90-8.15(2H, m); 8.85-9.15(2H, m); 10.17(1H, 2s broad); 10.26(1H, broad s) |
| 140 | 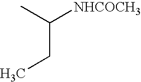 | (acetone-d6)=0.98(3H, m); 1.60-2.05(3H, 2s+2H, m); 3.89(3H, 2s); 4.42(1H, m); 6.90-7.20(4H, m); 7.35-7.50(2H, m); 7.75-7.95(1H, m); 8.35-8.55(1H, m); 9.55(1H, 2s broad) 10.35(1H, 2s broad) |
| 141 | 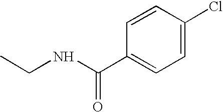 | (acetone-d6)=3.89(3H, 2s); 4.15(2H, m); 6.95-7.20(4H, m); 7.35-7.50(2H, m); 7.50-7.70(2H, m); 7.90-8.15(3H, m); 8.35-8.50(1H, m) |
| 142 | 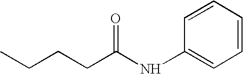 | (acetone-d6)=2.40-2.65(6H, m); 3.89(3H, 2s);6.95-7.20(4H, m); 7.20-7.45(4H, m); 7.60-7.75(3H.m); 7.75-7.95(2H, m); 9.20(1H, broad s); 9.43(1H, 2s broad) |
| 143 | 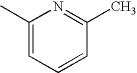 | (acetone-d6)=2.64(3H, s); 3.89(3H, 2s); 7.00-7.25(3H, m); 7.25-7.75(4H, m); 7.90-8.25(4H, m); 10.55(1H, broad s) |
| 144 | 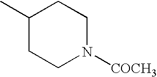 | (acetone-d6)=1.55-2.05(6H, m) 2.68(2H, m); 3.89(3H, 2s); 4.00(2H, m); 4.55(2H, m); 6.95-7.20(4H, m); 7.20-7.50(2H, m); 7.65-7.95(2H, m); 9.38(1H, 2s broad) |
| 145 | 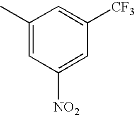 | (acetone-d6)=3.89(3H, 2s); 7.00-7.30(4H, m); 7.30-7.60(2H, m); 7.90-8.20(2H, m); 8.60-8.90(2H, m); 9.00-9.25(1H, m); 10.32(1H, 2s broad) |
| 146 | 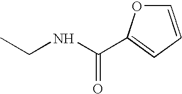 | (acetone-d6)=3.89(3H, 2s); 4.24(2H, dd, J=1.8Hz, J=5.8Hz); 6.40-6.75(1H, m); 6.90-7.20(5H, m); 7.30-7.50(2H, m); 7.65-8.00(3H, m); 8.42(1H, 2s broad); 9.59(1H, 2s broad) |
| 147 | 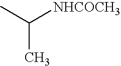 | (acetone-d6)=1.41(3H, m); 1.95(3H, m); 3.80-4.80(3H, 2s+1H, m); 6.95-7.20(3H, m); 7.20-7.60(3H, m); 7.75-7.95(1H, m); 8.30-8.55(1H, m); 9.56(1H, 2s broad); 10.36(1H, 2s broad) |
| 148 | 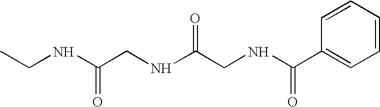 | (acetone-d6)=3.70-4.30(3H, 2s+6H, m); 6.85-7.65(10H, m); 7.85-8.10(2H, m) 8.35-8.50(1H, m) |
| 149 | 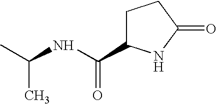 | (acetone-d6)=1.45(3H, d, J=5.76Hz) 1.55-2.55(4H, m); 3.89(3H, 2s); 4.25(1H, m); 4.59(1H, m); 6.80-7.60(7H, m); 7.80-8.10(1H, m); 8.40(1H, d, J=9.5Hz); 9.57(1H, 2s broad); 10.49(1H.s broad) |
| 150 | 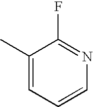 | (acetone-d6)=3.89(3H, 2s); 6.90-7.70(7H, m); 7.80-8.10(2H, m); 8.30-8.55(2H, m); 9.78(1H, 2s broad) |
| 151 | 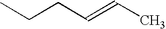 | (acetone-d6)=1.64(3H, m); 2.25-2.55(4H, m); 3.89(3H, 2s); 5.40-5.65(2H, m); 7.00-7.20(4H, m); 7.30-7.45(2H, m); 7.70-7.95(2H, m); 9.32(1H, 2s broad) |
| 152 | 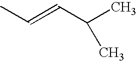 | (acetone-d6)=1.10(6H, m); 2.25-2.40(1H, m); 3.89(3H, 2s); 6.11(1H, d, J=15Hz); 6.80-7.00(1H, m) 7.00-7.20(4H, m); 7.30-7.50(2H, m); 7.75-7.95(2H, m) 9.40(1H, 2s broad) |
| 153 | 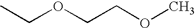 | (acetone-d6)=3.45(3H, s); 3.66(2H, m); 3.81(2H, m); 3.89(3H, 2s); 4.12(2H, s); 6.95-7.25(4H, m); 7.30-7.55(2H, m); 7.65-8.00(2H, m); 9.35(1H, 2s broad) |
| 154 | 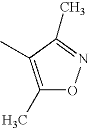 | (acetone-d6)=2.42(3H, s); 2.55(3H, s); 3.89(3H, 2s); 6.95-7.25(4H, m); 7.35-7.55(2H, m); 7.75-8.05(2H, m); 9.30(1H, 2s broad) |
| 155 | 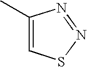 | (acetone-d6)=3.89(3H, 2s); 7.00-7.35(4H, m); 7.35-7.50(2H, m); 8.00-8.30(2H, m); 9.72(1H, m); 10.30(1H, 2s broad) |
| 156 | 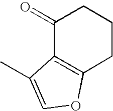 | (acetone-d6)=2.30(2H, m); 2.75(2H, m); 3.09(2H, m); 3.89(3H, 2s): 7.00-7.30(4H, m); 7.30-7.60(2H, m); 7.85-8.10(2H, m); 8.20-8.40(1H, d, J=4.3Hz); 12.11(1H, 2s broad) |
| 157 | 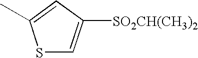 | (acetone-d6)=1.32(6H, d, J=6.75Hz); 3.37(1H, m); 3.89(3H, 2s); 7.00-7.30(4H, m); 7.30-7.55(2H, m); 7.85-8.10(2H, m); 8.10-8.30(1H, m); 8.40-8.60(1H, m); 10.04(1H, 2s broad) |
| 158 | 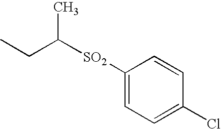 | (acetone-d6)=1.15-1.60(3H, m); 2.25-2.45(1H, m); 2.60-3.20(2H, m); 3.89(3H, 2s); 6.85-8.60(12H, m) |
| 159 | 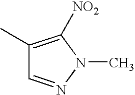 | (acetone-d6)=3.89(3H, 2s); 4.15(3H, s); 6.90-8.20(9H, m) |
| 160 | 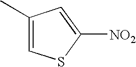 | (acetone-d6)=3.89(3H, 2s); 6.95-7.30(4H, m); 7.30-7.60(2H.m)7.85-8.10(2H, m); 8.45-8.80(2H, m); 9.89(1H, 2s broad) |
| 161 | 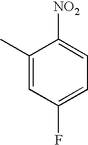 | (acetone-d6)=3.89(3H, 2s); 6.80-7.50(9H, m); 7.50-8.40(2H, m) |
| 162 | 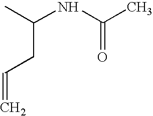 | (acetone-d6)=1.98(3H, s); 2.25-2.75(3H, m); 3.89(3H, 2s); 4.40-4.70(1H, m); 4.95-5.30(1H, m); .570-6.00(1H, m); 6.95-7.50(6H, m); 7.70-7.90(2H, m); 9.52(1H, 2s broad); 10.26(1H, broad s) |
| 163 | 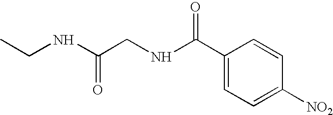 | (acetone-d6)=3.80-4.25(3H, 2s+2H, m+2H, m); 6.95-7.55(8H, m); 7.90-8.05(1H, m); 8.15-8.55(3H, m); 8.87(2H, broad s); 9.34(1H, 2s broad) |
| 164 | 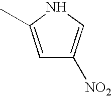 | (acetone-d6)=3.89(3H, 2s); 6.78-7.10(3H, m); 7.10-7.70(5H, m); 7.70-8.60(2H, m) |
| 165 | 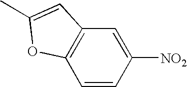 | (acetone-d6)=3.89(3H, 2s); 7.00-7.30(4H, m); 7.40-7.55(2H, m); 7.80-8.18(4H, m); 8.38-8.52(1H, m); 8.75-8.95(1H, m); 10.18(1H, 2s broad) |
| 166 | 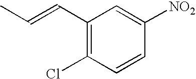 | (acetone-d6)=3.89(3H, 2s); 7.05-7.25(4H, m); 7.80-8.15(5H, m); 8.20-8.40(2H, m); 8.55-8.70(2H, m); 9.42(1H, 2s broad) |
| 167 | 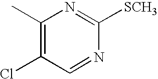 | (acetone-d6)=2.60(3H, s); 3.89(3H, 2s); 7.00-7.70(6H, m); 7.90-8.20(2H, m); 8.80-9.00(1H, m); 10.27(1H, 2s broad) |
| 168 | 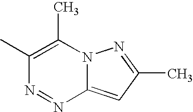 | (acetone-d6)=2.57(3H, s); 3.24(3H, s); 3.89(3H, 2s); 6.95-7.20(4H, m); 7.20-7.30(2H, m); 7.30-7.60(3H, m) 8.15(1H, 2s broad) |
| 169 | 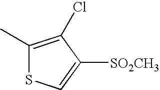 | (acetone-d6)=3.34(3H, s); 3.89(3H, 2s); 7.00-7.30(4H, m); 7.30-7.56(2H, m); 7.82-8.06(2H, m); 8.55-8.71(1H, m); 9.75(1H, 2s broad) |
| 170 | 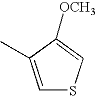 | (acetone-d6)=3.89(3H, 2s); 4.16(3H, s); 6.70-6.95(1H, m); 6.95-7.25(4H, m); 7.25-7.55(2H, m); 7.75-8.10(2H, m); 8.10-8.38(1H, m); 9.59(1H, 2s broad) |
| 171 | 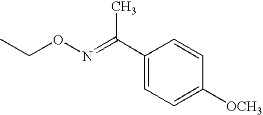 | (acetone-d6)=2.92(3H, s); 3.89(6H, 2s); 4.76(2H, m); 6.80-7.20(6H, m); 7.30-7.50(1H, m); 7.60-8.15(5H, m) |
| 172 | 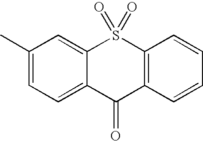 | (acetone-d6)=3.89(3H, 2s); 6.98-7.30(5H, m); 7.30-7.60(2H, m); 7.95-8.20(4H, m); 8.20-8.81(4H, m); 10.31(1H, 2s broad) |
| 173 | 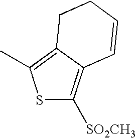 | (acetone-d6)=1.95-2.25(2H, m); 2.46(2H, m); 3.29(3H, s); 3.89(3H, 2s); 6.32-6.56(1H, m); 7.00-7.30(5H, m); 7.30-7.55(2H, m); 7.80-8.10(2H, m); 9.60(1H, 2s broad) |
| 174 | 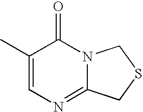 | (acetone-d6)=3.82(2H, m); 3.89(3H, 2s); 4.73(2H, m); 6.97-7.28(4H, m); 7.28-7.54(2H, m); 7.80-8.05(2H, m); 8.60-8.80(1H, m); 11.29(1H, 2s broad) |
| 175 | 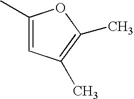 | (acetone-d6)=2.03(3H, s); 2.30(3H, s); 3.89(3H, 2s); 6.77-7.29(5H, m); 7.29-7.55(2H, m); 7.80-8.20(2H, m); 9.52(1H, 2s broad) |
| 176 | 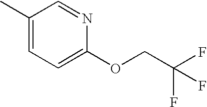 | (acetone-d6)=3.89(3H, 2s); 5.07(2H, m); 7.00-7.30(5H, m); 7.30-7.60(2H, m); 7.85-8.15(2H, m); 8.32-8.55(1H, m); 8.80-9.00(1H, m); 9.84(1H, 2s broad) |
| 177 | 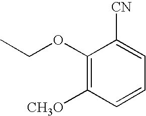 | (acetone-d6)=3.89(3H, 2s); 4.01(3H, s); 4.86(2H, m); 7.02-7.30(3H, m); 7.30-7.57(6H, m); 7.85-8.12(2H, m); 9.48(1H, 2s broad) |
| 178 | 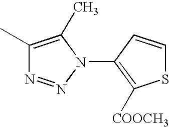 | (acetone-d6)=2.56(3H, 2s); 3.62(3H, s); 3.89(3H, 2s); 7.00-7.30(5H, m); 7.30-7.60(3H, m); 8.05-8.25(2H, m); 9.80(1H, 2s broad) |
| 179 | 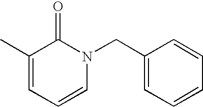 | (acetone-d6)=3.82(3H, 2s); 5.43(2H, s); 6.60-6.80(1H, m); 6.99-7.25(3H, m); 7.25-7.59(8H, m); 7.80-8.07(2H, m); 8.07-8.30(1H, m); 8.50-8.75(1H, m); 12.39(1H, 2s broad) |
| 180 | 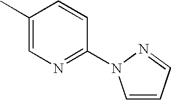 | (acetone-d6)=3.89(3H, 2s); 6.55-6.71(1H, m); 7.02-7.29(3H, m); 7.37-7.60(1H, m); 7.80-8.26(5H, m); 8.50-8.80(3H, m); 9.00-9.25(1H, m); 9.98(1H, 2s broad) |
| 181 | 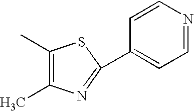 | (acetone-d6)=2.79(3H, s); 3.89(3H, 2s); 6.94-7.29(2H, m); 7.90-8.20(6H, m); 8.70-9.97(4H, m) |
| 182 | 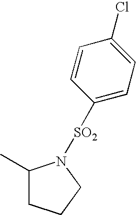 | (acetone-d6)=1.35-1.50(1H, m); 1.55-2.10 (3H, m); 3.25-3.75(2H.m); 3.89(3H, 2s); 4.30(1H, m); 7.00-7.25(4H, m); 7.35-7.55(2H m); 7.67-8.15(6H, m); 9.54(1H, 2s broad) |
| 183 | 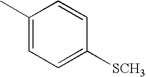 | (acetone-d6)=2.60(3H, s); 3.89(3H, 2s); 6.92-7.30(4H, m); 7.30-7.60(4H, m); 7.85-8.20(4H, m); 9.71(1H, 2s broad) |
| 184 | 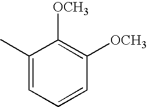 | (acetone-d6)=3.89(3H, 2s); 3.98(3H, 2s); 4.03(3H, 2s); 7.00-7.40(6H, m); 6.40-6.53(2H, m); 6.53-7.70(1H, m); 7.90-8.15(2H, m); 10.15(1H, 2s broad) |
| 185 | 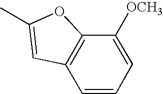 | (acetone-d6)=3.89(3H, 2s); 4.08(3H, 2s); 6.95-7.55(9H, m); 7.55-7.85(1H, m); 7.85-8.22(2H, m) |
| 186 | 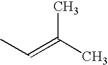 | (acetone-d6)=1.91(3H, s); 2.25(3H, s); 3.89(3H, 2s); 5.92(1H, m); 6.90-7.20(4H, m); 7.30-7.50(2H, m); 7.72-8.07(2H, m); 9.26(1H, 2s broad) |
| 187 | 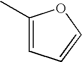 | (acetone-d6)=3.89(3H, 2s); 6.60-6.80(1H, jm); 7.00-7.21(4H, m); 7.21-7.35(1H, m); 7.35-7.55(2H, m); 7.73-7.90(1H, m); 7.90-8.15(2H, m); 9.63(1H, 2s broad) |
| 188 | p-tosyl | (acetone-d6) = 3.22(3H, s); 3.89(3H, 2s); |
| | | 6.90-7.30(5H, m); 7.37-7.60(2H, m); |
| | | 7.95-8.20(3H, m); 8.20-8.40(2H, m); |
| | | 9.96(1H, 2s broad) |
| 189 | 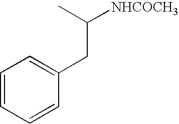 | (acetone-d6)=1.28(3H, s); 1.75-1.90(2H, m); 3.89(3H.2s); 4.10(1H, m); 6.80-7.85(13H, m) |
| 190 | 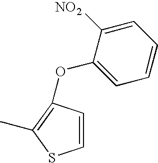 | (acetone-d6)=3.89(3H, 2s); 6.90-7.30(4H, m); 7.30-7.70(4H, m); 7.70-8.05(4H, m); 8.05-8.30(2H, m); 9.43(1H, 2s broad) |
| 191 | 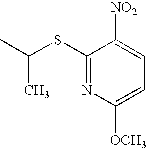 | (acetone-d6)=1.73(3H, 2xd, J=6.93Hz); 3.89(3H, 2s); 4.10(3H, 2s); 4.68(1H, q, J=6.93Hz); 6.65-6.90(2H, m); 7.00-7.25(4H, m); 7.30-7.51(2H, m); 7.65-8.00(2H, m); 9.68(1H, 2s broad) |
| 192 | 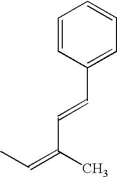 | (acetone-d6)=1.85(3H, m); 3.89(3H, 2s); 5.6(1H, m); 6.70-8.10(14H, m); 8.42(1H, m); 10.2(1H, 2s broad) |
| 193 | 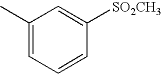 | (acetone-d6)=3.23(3H, s); 3.89(3H, 2s); 6.98-7.30(3H, m); 7.30-7.54(2H, m); 7.76-8.10(4H m); 8.10-8.30(1H, m); 8.30-8.45(1H, m); 8.45-8.65(1H, m); 10.14(1H, 2s broad) |
| 194 | 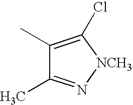 | (acetone-d6)=2.40(3H, s); 3.84(3H, s); 3.89(3H, 2s); 7.00-7.27(4H, m); 7.27-7.55(2H, m); 7.80-8.10(2H, m); 9.09(1H, 2s broad) |
| 195 | 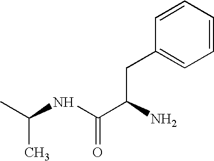 | (acetone-d6)=1.25(3H, m); 3.1-3.65(2H, m); 3.89(3H, 2s); 4.06(1H, m); 4.5(1H.m); 6.60-7.45(11H, m); 7.60-8.00(2H, m); 8.36(1H, 2s broad) |
| 196 | 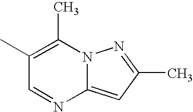 | (acetone-d6)=2.55(3H, s); 2.74(3H, s); 3.89(3H, 2s); 6.25-8.45(10H, m) |
| 197 | 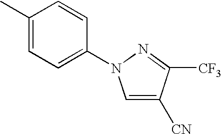 | (acetone-d6)=3.89(3H, 2s); 7.00-7.30(3H, m); 7.30-7.59(1H, m); 7.71-7.92(3H, m); 7.92-8.15(2H, m); 8.15-8.40(3H, m); 8.40-8.60(1H.m); 10.01(1H, 2s broad) |
| 198 | 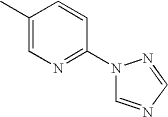 | (acetone-d6)=3.89(3H, 2s); 6.90-7.31(6H, m); 7.31-7.75(4H, m); 7.95-8.50(3H, m) |
| 199 | 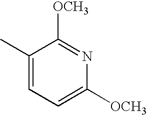 | (acetone-d6)=3.89(3H, 2s); 4.04(3H, s); 4.25(3H, s); 6.40-6.75(1H, m); 6.97-7.27 (4H, m); 7.28-7.56(2H, m); 7.80-8.15(2H, m); 8.30-8.60(1H, m); 9.93(1H, 2s broad) |
| 200 | 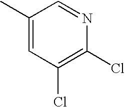 | (acetone-d6)=3.89(3H, 2s); 6.95-7.30(4H, m); 7.36-7.57(2H, m); 7.89-8.11(2H, m); 8.50-8.70(1H, m); 8.90-9.10(1H, m); 10.05(1H, 2s broad) |
| 201 | 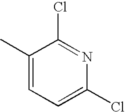 | (acetone-d6)=3.89(3H, 2s); 6.20-7.58(7H, m); 7.58-8.70(3H, m) |
| 202 | 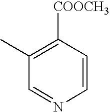 | (acetone-d6)=3.89(3H, 2s); 4.13(3H, s); 5.93-8.14(8H, m); 8.14-10.08(3H, m) |
| 203 | 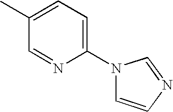 | (acetone-d6)=3.89(3H, 2s); 7.00-7.33(3H, m); 7.33-7.54(3H, m); 7.90-8.40(5H, m); 8.60-8.80(1H, m); 9.01-9.30(2H, m); 10.05(1H, 2s broad) |
| 204 | 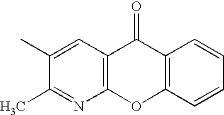 | (acetone-d6)=2.87(3H, s); 3.89(3H, 2s); 6.11-7.43(4H, m); 7.43-7.90(4H, m); 7.90-8.64(4H, m); 8.64-9.89(1H, m) |
| 205 | 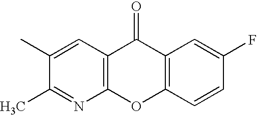 | (acetone-d6)=3.30(3H, s); 3.89(3H, 2s); 6.15-7.40(6H, m); 7.40-8.60(6H, m) |
| 206 | 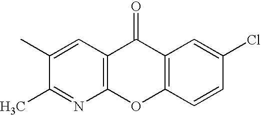 | (acetone-d6)=2.52(3H, s); 3.89(3H, 2s); 6.15-7.65(8H, m); 7.65-9.00(4H, m) |
| 207 | 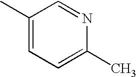 | (acetone-d6)=2.68(3H, s); 3.89(3H, 2s); 6.68-7.74(8H, m); 7.74-8.20(1H, m); 8.30-8.60(1H, m); 8.94-9.31(1H, m); 9.94(1H, broad s) |
| 208 | 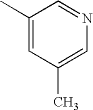 | (acetone-d6)=2.32(3H, s); 3.89(3H, 2s); 6.55-7.50(7H, m); 7.81-8.40(2H, m); 8.47-8.70(1H, m); 8.70-9.07(1H, m); 9.76(1H, 2s broad) |
| 209 | 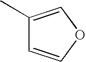 | (acetone-d6)=3.89(3H, 2s); 6.90-7.30(5H, m); 7.30-7.60(2H, m); 7.60-7.85(1H, m); 7.85-8.15(2H, m); 8.15-8.50(1H, m) |
| 210 | 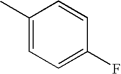 | (acetone-d6)=3.89(3H, 2s), 6.90-7.21(4H, m); 7.21-7.55(4H, m); 7.90-8.30(4H, m) 9.77(1H, 2s broad) |
| 211 | 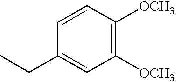 | (acetone-d6)=3.70-3.75(2H, m); 3.82(6H, 2s); 3.89(3H, 2s); 6.80-7.00(4H, m); 7.00-7.19(4H, m); 7.30-7.43(1H, m); 7.70-7.90(2H, m); 9.43(1H, 2s broad) |
| 212 | 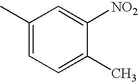 | (acetone-d6)=2.68(3H, s); 3.89(3H, 2s); 7.00-7.30(4H, m); 7.30-7.59(2H, m); 7.59-7.86(1H, m); 7.86-8.15(2H, m); 8.15-8.42(1H, m); 8.42-8.70(1H, m); 10.03(1H, 2s broad) |
| 213 | 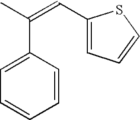 | (acetone-d6)=3.89(3H, 2s); 6.94-7.02(4H, m); 7.20-7.55(6H, m); 7.55-7.70(3H, m); 7.70-7.91(2H, m); 7.91-8.19(1H, m); 7.29-7.40(1H, m) |
| 214 | 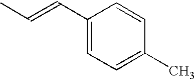 | (acetone-d6)=2.39(3H, s); 3.89(3H, 2s); 6.70-7.00(2H, m); 7.00-7.20(2H, m); 7.20-7.37(3H, m); 7.37-7.50(1H, m); 7.50-7.80(4H, m); 7.80-8.10(2H, m) |
| 215 | 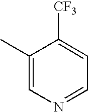 | (acetone-d6)=3.89(3H, 2s); 6.20-8.10(9H, m); 8.10-10.02(2H, m) |
| 216 | 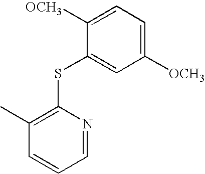 | (acetone-d6)=3.55-4.10(9H, 3x2s); 6.15-7.80(12H, m); 7.80-8.70(2H, m) |
| 217 | 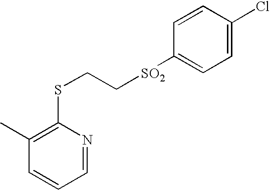 | (acetone-d6)=3.20-4.30(7H, m); 6.30-7.60(8H, m); 7.60-8.80(7H, m) |
| 218 | 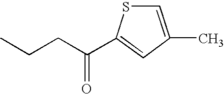 | (acetone-d6)=2.32(3H, s); 2.73(2H, t, J=6.64Hz); 3.27(2H, t, J=6.64Hz); 3.89(3H, 2s); 6.80-8.20(10H, m): 10.45(1H, broad s) |
| 219 | —CH₂—S—C₁₂H₂₅ | (acetone-d6)=0.90(3H, m); 1.05-1.85(20H, m); |
| | | 2.73(2H, m); 3.26(2H, s); 3.89(3H, 2s); |
| | | 6.10-7.25(4H, m); 7.25-7.60(2H, m); |
| | | 7.60-8.70(2H, m); 9.87(1H, 2s broad) |
| 220 | 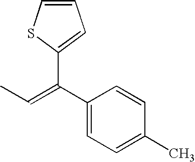 | (acetone-d6)=1.28(3H, s); 1.89(3H, 2s); 6.30-8.25(16H, m); 8.36(1H, 2s broad) |
| 221 | 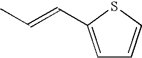 | (acetone-d6)=3.89(3H, 2s); 6.50-6.80(1H, m); 6.95-7.25(5H, m); 7.25-7.55(3H, m); 7.55-7.75(1H, m); 7.75-8.10(3H, m); 9.56(1H, broad s) |
| 222 | 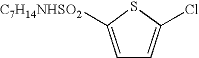 | (acetone-d6)=1.02-1.85(10H, m); 2.40(2H, m); 3.14(2H, m); 3.89(3H, 2s); 6.45-6.70(1H, broad s); 6.70-6.95(1H, m); 6.95-7.30(4H, m); 7.30-7.60(3H, m); 7.60-8.10(2H, m); 9.30(1H, broad s) |
| 223 | 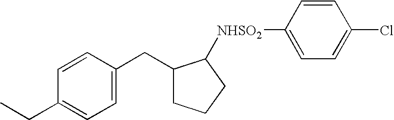 | (acetone-d6)=1.20-1.95(6H, m); 2.25-2.55(2H, m+1H, m); 3.60(2H, m); 3.89(3H, 2s); 4.38(1H, m); 6.95-7.20(5H, m); 7.20-7.45(3H, m); 7.60-7.75(3H, m); 7.75-8.05(5H, m); 9.47(1H, 2s broad) |
| 224 | 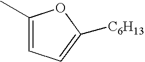 | (acetone-d6)=0.91(3H, m); 1.2-1.85(8H, m); 2.60-2.90(2H, m); 3.89(3H, 2s); 6.90-7.30(4H, m); 7.30-7.65(4H, m); 7.80-8.20(2H, m); 9.57(1H, 2s broad) |
| 225 | 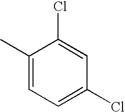 | (acetone-d6)=3.89(3H, 2s); 6.30-8.70(11H, m) |
| 226 | 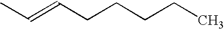 | (acetone-d6)=0.92(3H, m); 1.25-1.70(6H, m); 2.20-2.40(2H, m); 3.89(3H, 2s); 6.00-6.30(1H, m); 6.75-7.25(5H, m); 7.25-7.55(2H, m); 7.7-8.1(2H, m) |
| 227 | 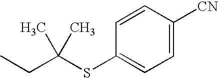 | (acetone-d6)=1.54(6H, m); 2.73(2H, 2s); 3.89(3H, 2s); 7.00-7.25(4H, m); 7.25-7.50(2H, m); 7.70-7.95(6H, m); 10.26(1H, 2s broad) |
| 228 | 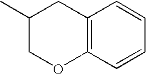 | (acetone-d6)=2.80-3.40(3H, m); 3.87(3H, 2s); 4.00-4.65(2H, m); 6.65-6.95(3H, m); 6.95-7.25(6H, m); 7.25-7.50(1H, m); 7.70-8.00(2H, m); 9.64(1H, 2s broad) |
| 229 | 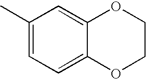 | (acetone-d6)=3.89(3H, 2s); 4.25-4.55(4H, m); 6.80-7.30(5H, m); 7.30-7.75(4H, m); 7.75-8.20(2H, m); 9.58(1H, 2s broad) |
| 230 | 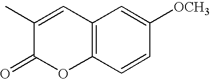 | (acetone-d6)=3.89(3H, 2s); 3.95(3H, 2s); 7.00-7.30(3H, m); 7.40-7.70(6H, m); 7.90-8.10(2H, m); 9.70(1H, m); 11.04(1H, 2s broad) |
| 231 | 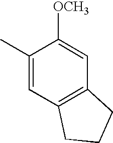 | (acetone-d6)=1.42(2H, m); 2.95(4H, m); 3.90(3H, 2s); 4.10(3H, 2s); 7.05-7.20(5H, m); 7.40-7.55(2H, m); 7.90-8.10(3H, m); 10.20(1H, 2s broad) |
| 232 | 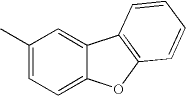 | (acetone-d6)=3.89(3H, 2s); 6.60-7.60(9H, m); 7.60-8.40(5H, m); 8.50-8.85(1H, m); 10.1(1H, s) |
| 233 | 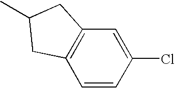 | (acetone-d6)=2.90-3.55(4H, m); 3.89(3H, 2s); 3.90-4.10(1H, m); 6.80-7.20(7H, m); 7.20-7.30(2H, m); 7.55-7.85(2H, m); 9.40(1H, 2s broad) |
| 234 | 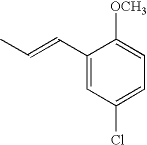 | (acetone-d6)=3.89(3H, 2s); 3.94(3H, s); 6.8-7.05(6H, m); 7.2-7.35(3H, m); 7.41-7.50(1H, m); 7.70-7.85(3H, m); 9.45(1H, 2s broad) |
| 235 | 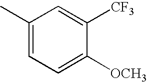 | (acetone-d6)=3.89(3H, 2s); 4.01(3H, s); 6.85-7.10(3H, m); 7.20-7.35(3H, m); 7.70-7.95(2H, m); 8.05-8.30(3H, m); 9.7(1H, 2s broad) |
| 236 | 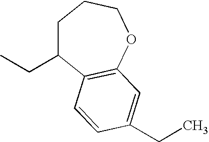 | (acetone-d6)=0.95(3H, m); 1.55-1.75(2H, m); 2.30-2.45(2H, m); 3.30-3.65(5H, m); 3.89(3H, 2s), 3.90-4.30(2H, m); 6.60-7.05(6H, m); 7.10-7.30(2H, m); 7.50-7.85(3H, m); 9.20(1H, 2s broad) |
| 237 | 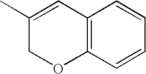 | (acetone-d6)=3.89(3H, 2s); 4.92(2H, 2s), 6.60-7.05(6H, m); 7.05-7.35(5H, m); 7.60-7.90(2H, m); 9.40(1H, 2s broad) |
| 238 | 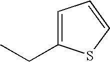 | (acetone-d6)=3.75-4.05(2H, s+3H, 2s); 6.80-7.15(4H, m); 7.15-7.50(4H, m) 7.50-7.95(3H, m) |
| 239 | 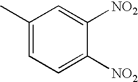 | (acetone-d6)=3.89(3H, 2s); 6.70-8.05(8H, m); 8.15-8.80(3H, m) |
| 240 | 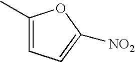 | (acetone-d6)=3.90(3H, 2s); 7.00-7.30(4H, m); 7.30-7.60(3H, m); 7.60-7.80(1H, m); 7.90-8.15(2H, m); 10.35(1H, 2s broad) |
| 241 | 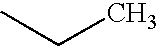 | (acetone-d6)=1.18(3H, m); 2.40(2H, m); 3.89(3H, 2s); 7.00-7.25(4H, m); 7.25-7.50(2H, m); 7.70-7.95(2H, m); 9.36(1H, 2s broad) |
| 242 | 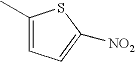 | (acetone-d6)=3.90(3H, 2s); 7.05-7.30(3H, m); 7.30-7.60(2H, m); 7.90-8.20(5H, m); 10.14(1H, 2s broad) |

### EXAMPLE 242 a

Compound of the formula I: T=4-OMe; X=H; A=CO; Y=4-methoxybenzyl

Step a

Corresponding Compound of the Formula II

0.103 g (0.405 mmol) of bis(2-oxo-3-oxazolidinyl)phosphonyl chloride dissolved in 3.7 ml of a dichloroethane/DMF solution (9:1) is added to a solution of 68.1 mg (0.405 mmol) of 4-methoxyphenylacetic acid and 0.113 ml (0.81 mmol) of triethylamine in 3 ml of dichloroethane, followed by addition of 133 mg (i.e. 0.081 mmol) of resin prepared in Example 11a). After stirring for 4 hours at 60° C., the resin is filtered off and washed successively with a dichloromethane/dichloroethane mixture (4:1), with a DMF/water mixture (1:1), with DMF and then with dichloromethane/dichloroethane (4:1).

The resin is suspended in 1 ml of a dichloromethane/dichloroethane mixture (4:1) containing 1.5 ml of a dichloromethane/trifluoroacetic acid mixture (1:1). After stirring for 3 hours at room temperature, the resin is filtered off and washed with dichloromethane. The filtrate, concentrated under vacuum, gives 9 mg of the expected product (yield=31%).

NMR (DMSO-d₆) δ (ppm)=3.6 (3H, s); 3.65 (3H, s); 6.8-7.0 (6H, m); 7.0-7.15 (2H, m); 7.2 (2H, m); 7.3 (2H, m); 9.5 (1H, broad s); 9.8 (1H, s).

An identical result is obtained by replacing the 4 hours of heating at 60° C. with 15 hours of stirring at room temperature.

This reaction can be carried out replacing the dichloromethane/DMF (9:1) reaction solvent with diglyme and heating for 4 hours at 120° C.

Step b

Title Compound

This compound is obtained by performing the process as in Example 4, starting with the compound obtained in the above step a).

NMR (acetone-d₆)=3.6 (2H, s); 3.8 (3H, s); 4.9 (3H, 2s); 6.9 (2H, d, J=8.6 Hz); 7.05 (4H, m); 7.3 (4H, m); 7.75 (1H, m); 9.45 (1H, 2s, exchangeable with CF₃COOD).

The compounds of Table 7 are compounds of the formula I in which X=H and A=CO.

TABLE 7

| Example | | | |
|---|---|---|---|
| No. | T | Y | NMR |
| 243 | H | 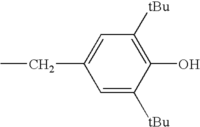 | (CDCl3) = 1.4-1.6 (18H, s); 3.66 (2H, m); 7.85-6.35 (12H, m) |
| 244 | 4-OCH₃ | 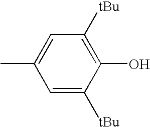 | (acetone-d6) = 1.47 (18H, s); 3.86 (3H, 2s); 6.90-7.20 (4H, m); 7.30-7.45 (2H, m); 7.75-8.00 (5H, m); 9.65 (1H, 2s broad) |
| 244 a | 4-OCH₃ | 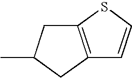 | (acetone-d6) = 2.65-3.30 (5H, m) 3.89 (3H, 2s) 6.65-7.15 (5H, m); 7.15-7.45 (3H, m); 7.70-7.90 (2H, m); 9.50 (1H, 2s broad) |
| 245 | 4-OCH₃ | 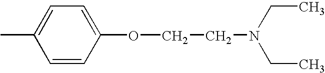 | (acetone-d6) = 1.20-1.60 (6H, m) 3.35-3.65 (4H, m); 3.80-4.00 (3H, 2s + 2H, m); 4.20-4.35 (2H, m); 6.75-7.20 (6H, m); 7.25-7.5 (2H, m); 7.75-8.40 (4H, m); 9.7 (1H, 2s broad) |
| 246 | 4-OCH₃ | 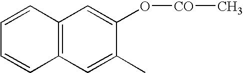 | (acetone-d6) = 2.25 (3H, s); 3.89 (3H, 2s); 6.65-8.15 (14H, m); 11.35 (1H, 2s broad) |
| 247 | 4-OCH₃ | 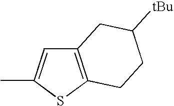 | (acetone-d6) = 1.2 (9H, s) 1.35-1.55 (3H, m); 1.95-2.45 (2H.m); 2.45-2.95 (2H, m); 3.89 (3H, 2s); 6.75-7.15 (4H, m); 7.15-7.40 (2H, m); 7.40-7.65 (1H, m); 7.65-7.95 (2H.m) 9.45 (1H, 2s broad) |
| 248 | 4-OCH₃ | 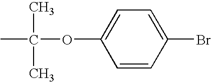 | (acetone-d6) = 1.60 (6H, s); 3.90 (3H, 2s); 6.80-7.20 (5H, m); 7.20-7.70 (5H, m); 7.70-8.10 (2H, m); 9.60 (1H, 2s broad) |
| 249 | 4-OCH₃ | 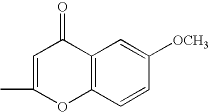 | (acetone-d6) = 3.75 (3H, 2s); 4.96 (3H, 2s); 6.31-8.21 (12H, m); 10.22 (1H, 2s broad) |
| 250 | 4-OCH₃ | 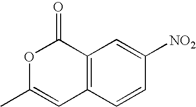 | (acetone-d6) = 3.89 (3H, 2s); 6.42-7.32 (4H, m); 7.32-7.62 (1H, m); 7.62-7.95 (2H, m); 7.95-8.32 (3H, m); 8.62-9.12 (2H, m); 10.27 (1H, 2s broad) |
| 251 | 4-OCH₃ | 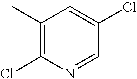 | (DMSO-d6) = 4.32 (3H, 2s); 7.45-7.70 (4H, m); 7.80-7.95 (2H, m); 8.25-8.45 (2H, m); 8.60-8.75 (1H, m); 8.9-9.05 (1H, m); 10.40 (1H, 2s broad) |

### EXAMPLE 252

The compounds of the invention increase the nitric oxide level.

A solution of a compound of the invention spontaneously releases nitric oxide. The nitrite ions resulting therefrom are titrated by colorimetry by means of a specific reagent (Griess). To take account of any release of nitrate ions in addition to the nitrite ions, bacterial nitrate reductase is added to the reaction medium to allow the nitrate ions formed to be reduced.

The reactions and measurements are carried out in transparent 96-well plates. The test products are dissolved at the time of use to a concentration of 3 mM in dimethyl sulfoxide. 95 μl of a reagent containing nitrate reductase (0.18U/ml in 100 mM pH 7.5 PBS buffer, 210 μM β-NADPH, 5 μM FAD) and 5 μl of a solution of the test product (final concentration of 150 μM) are then added to each well. After stirring, the mixture is left to incubate for 4 hours at 37° C. The reaction is then quenched by addition of 100 μl of the Griess reagent (Sigma G4410). The reagent is allowed to act for 5 minutes at room temperature and the optical density is then read at 540. nm. This value is proportional to the concentration of nitrites+nitrates in the medium. A calibration range is made for each plate using NaNO₂.

The results are expressed as pmol/I (μM) of nitrites+nitrates released, in Table 5 below.

The compounds of the invention reduce the biological activity of oxidative free-radical species.

Human LDLs dissolved in aqueous solution in the presence of cupric ion become spontaneously oxidized on their protein component, apolipoprotein B. This oxidation makes the particle fluorescent, which is exploited to measure a pharmacological effect.

The reactions and measurements are carried out in black 96-well plates, 10 μl of a solution of the test product dissolved in dimethyl sulfoxide are first mixed with 170 μl of a solution of human LDL at 120 μg/ml and 20 μl of 100 μM CuCl₂. After stirring, the mixture is left to incubate for 2 hours at 37° C., and a first fluorescence reading is taken (excitation at 360 nm, reading at 460 nm). The mixture is then left to incubate for a further 22 hours, to take a second reading under the same conditions. The difference between the two values obtained is the measurement of the oxidation of the LDLs in solution. This difference is proportionately smaller the greater the antioxidant power of the test product. Probucol is used as reference product at 10 μM.

The concentrations which inhibit the oxidation by 50% (IC₅₀) are produced from 3 product concentrations. They are given in Table 8 below.

TABLE 8

| Example | Nitrates + nitrites | IC₅₀ Antioxidant |
|---|---|---|
| No. | (μM) | effect (μM) |
| 57 | 109 | 8.9 |
| 1 | 97 | 6.4 |
| 12 | 22 | 2.2 |
| 8 | 44 | 5.4 |
| 45 | 62 | 3.2 |
| 4 | 84 | 5.4 |
| 18 | 71 | 3.7 |
| 6 | 83 | 6.5 |
| 86 | 79 | 3.8 |
| Comparative | — | 6.4 |
| example: | | |
| Probucol | | |

### EXAMPLE 253

The carbamate subfamily: synthetic route

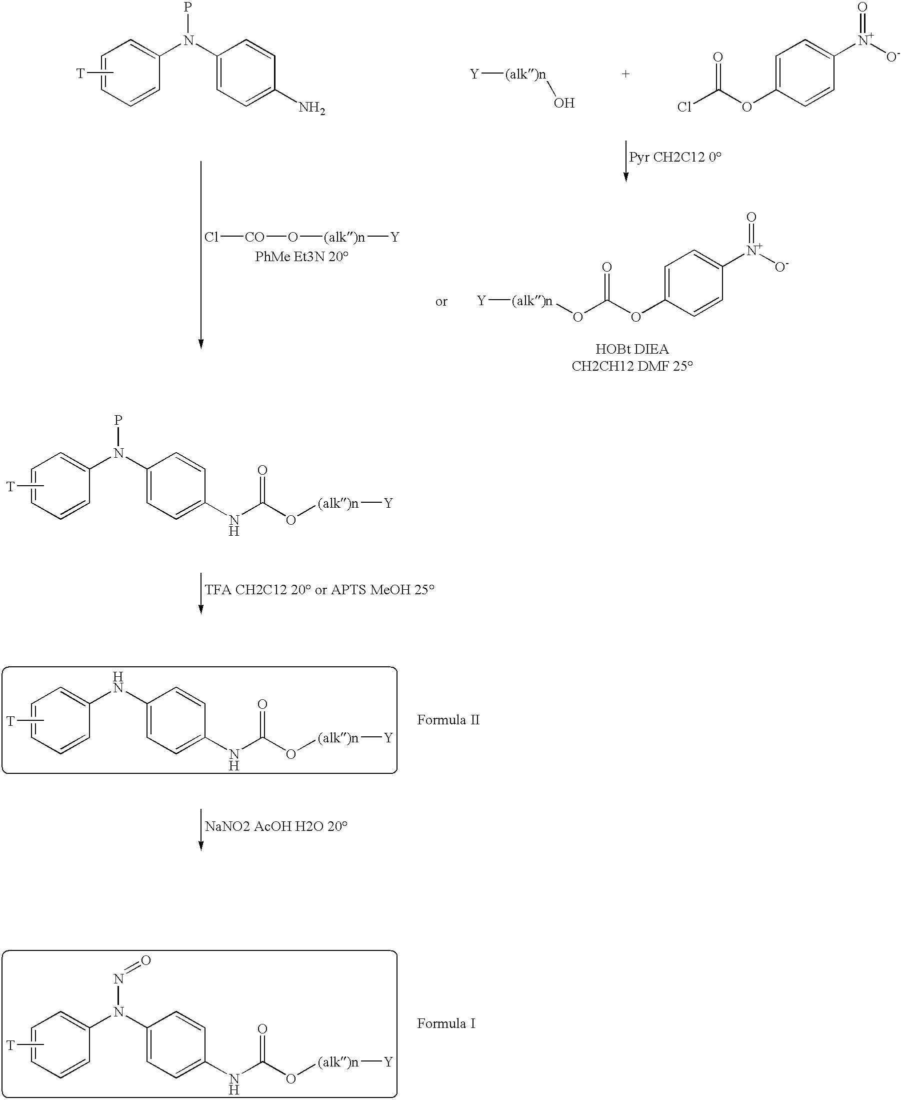

Benzyl 4-[1-(4-methoxyphenyl)-2-oxohydrazino]phenylcarbamate

Step a:

Benzyl 4-[N-(4-methoxyphenyl)-N-(tert-butoxycarbonyl)amino]phenylcarbamate

6.8 g (40 mmol) of benzyl chloroformate and 2 g (20 mmol) of triethylamine are added dropwise to a solution of 6.29 g (20 mmol) of 4-amino-4′-methoxy-N-butoxycarbonyidiphenylamine in 300 ml of toluene. After stirring at room temperature and checking that the starting material has disappeared by TLC on silica (1:2 ethyl acetate/heptane), the reaction medium is filtered and then concentrated under vacuum and the residue is taken up in dichloromethane. After washing with water (3×100 ml) and drying over Na₂SO₄, the organic phase is filtered and concentrated under vacuum to give 6.8 g of a product, which is used directly in the next step.

(Yield=76.4%)

NMR (CDCl₃)=1.35 (9H, s); 3.7 (3H, s); 5.1 (2H, s); 6.7 (1H, broad s); 6.7-6.8 (2H, m); 7.0-7.5 (11H, m).

Step b:

Benzyl 4-[(4-methoxyphenyl)amino]phenylcarbamate

The 6.8 g (15 mmol) of the compound obtained in step a) are taken up in a mixture of trifluoroacetic acid (40 ml) and dichloromethane (160 ml). After stirring for 1 hour at room temperature, the violet solution obtained is poured into a mixture of ice and 1N sodium hydroxide (final pH of between 7 and 8). The organic phase, separated out after settling the phases, is washed with water to neutral pH, dried over Na₂SO₄ and concentrated under vacuum to give a grey solid which, after trituration in isopropyl ether and drying, gives 3.3 g of solid.

(Yield=62.4%)

NMR (DMSO-d6): 3.85 (3H, s); 5.3 (2H, s); 6.95-7.3 (6H, m); 7.4-7.6 (8H, m); 9.8 (1H, broad s).

Step c:

Benzyl 4-[1(4-methoxyphenyl)-2-oxohydrazino]phenylcarbamate

This compound is obtained by performing the process as in Example 1, step c) starting with the compound obtained in the above step b).

(Yield=79%)

NMR (DMSO-d6): 3.6 (3H, 2s); 5.3 (2H, 2s); 7.1 (4H, m); 7.3 -7.45 (7H, m); 7.55-7.7 (2H, m); 10.0 (1H, 2s).

The pharmacological activity of the products of Example 253 (formula I and formula II):

T=4-MeO X=H A=CO Y=O—CH₂-Ph Formula 1,

Table 8:

Nitrites+nitrates (μM)=45

IC₅₀ Antioxidant effect (μM): 3.6

T=4-MeO X=H A=CO Y=O—CH₂ Ph Formula 2,

IC₅₀ (μM)=2.0.

### EXAMPLE 254

### Phenyl 4-[1-(4-methoxyphenyl)-2-oxohydrazino]phenylcarbamate

Obtained by performing the process as in Example 253, steps a), b) and c), starting with phenyl chloroformate.

### a) Phenyl 4-[N-(4-methoxyphenyl)-N-(tert-butoxycarbonyl)amino]phenylcarbamate

(Yield=69.2%)

NMR (CDCl₃)=1.5 (9H, s); 3.9 (3H, s); 6.9 (2H, m); 7.0 (1H, broad s); 7.15-7.30 (7H, m); 7.4-7.5 (4H, m).

### b) Phenyl 4-[(4-methoxyylhenyl)amino]phenylcarbamate

(Yield=38.5%)

NMR (CDCl₃)=3.7 (3H, s); 5.3 (1H, broad s); 6.6-6.85 (5H, m); 6.9-7.0 (2H, m); 7.05-7.35 (7H, m).

### c) Phenyl 4-[1-(4-methoxyphenyl)-2-oxohydrazino]phenylcarbamate

(Yield=78.7%)

NMR (acetone-d6)=3.7 (3H, 2s); 6.9-7.0 (4H, m); 7.0-7.4 (7H, m); 7.6-7.7 (2H, m) 9.4 (1H, 2 broad s).

### EXAMPLE 255

### 2-(Nitrooxy)ethyl 4-[1-(4-methoxyphenyl)-2-oxohydrazino]phenylcarbamate

### a) 2-Bromoethyl 4-[N-(4-methoxyphenyl)-N-(tert-butoxycarbonyl)amino]phenylcarbamate

Obtained by performing the process as in Example 254a starting with 2-bromoethyl chloroformate.

(Yield=82%)

NMR (CDCl₃): 1.3 (9H, s); 3.4 (2H, m); 3.7 (3 H, s); 4.4 (2H, m); 6.8 (3H, m); 7.0-7.3 (6H, m).

### b) 2-Bromoethyl [(4-methoxyphenyl)amino]phenylcarbamate

Obtained by performing the process as in Example 154b.

(Yield=56.2%)

NMR (DMSO-d6)=3.6 (4H, m); 3.8 (1H, t, J=5.4 Hz); 4.25 (1H, t, J=5.2 Hz); 4.35 (1H, t, J=5.4 Hz); 6.7-6.9 (6H, m); 7.2 (2H, m); 7.6 (1H, broad s); 9.5 (1H, broad s).

### c) 2-(Nitrooxy)ethyl 4-[(4-methoxyphenyl)amino]phenylcarbamate

A mixture of 0.3 g (0.8 mmol) of the compound obtained in Example 255b, 0.2 g (1.2 mmol) of silver nitrate and 20 ml of acetonitrile is stirred for 24 hours at room temperature in the absence of light. After filtering off a white precipitate, the solution is concentrated under vacuum and the residue is taken up in an ethyl acetate/water mixture. The organic phase is separated out after settling of the phases and is dried over Na₂SO₄ and then concentrated. The residue obtained is purified by flash chromatography on a column of silica in a heptane/ethyl acetate mixture (1:3) to give 0.1 g of solid.

(Yield=35%)

NMR (DMSO-d6)=3.7 (3H, s); 4.4 (2H, m); 4.8 (2H, m); 6.8 -6.9 (4H, m); 6.95 (2H, m) 7.2 (2H, m); 7.7 (1H, s); 9.5 (1H, broad s).

d) 2-(Nitrooxy)ethyl 4-[1-(4-methoxyphenyl)-2-oxohydrazino]phenylcarbamate

Obtained by performing the process as in Example 4.

(Yield=78%)

NMR (DMSO-d6)=3.8 (3H, 2s); 4.4 (2H, m); 4.8 (2H, m); 7.0 -7.2 (4H, m); 7.35 (2H, m); 7.5-7.7 (2H, m); 10.0 (1H, broad s).

### EXAMPLE 256

### Pyrid-3-ylmethyl 4-[1-(4-methoxyphenyl)-2-oxohydrazino]phenylcarbamate

### a) 4-Nitrophenyl pyrid-3ylmethylcarbonate

1 g (4.96 mmol) of 4-nitrophenyl chloroformate dissolved in 15 ml of CH₂Cl₂ is added at 0° C. to a solution of 0.54 g (4.96 mmol) of pyrid-3-ylmethanol and 0.59 g (7.44 mmol) of pyridine in 15 ml of CH₂Cl₂. After stirring for 2 hours at 0° C., the mixture is allowed to warm to room temperature and 150 ml of water are added.

The organic phase is separated out after settling of the phases, and the aqueous phase is extracted with CH₂Cl₂ (4×50 ml). The combined organic phases are washed with saturated aqueous NaCl solution, dried over Na₂SO₄ and concentrated under vacuum to give 0.81 g of a yellow solid.

(Yield=59.5%)

NMR (DMSO-d6): 5.5 (2H, s); 7.6-7.7 (1H, m); 7.7-7.85 (2H, m); 8.0-8.1 (1H, d, J=9.1 Hz); 8.45-8.6 (2H, m); 8.7-8.8 (1H, m); 8.85 (1H, s).

b) Pyrid-3-ylmethyl 4-[N-(4-methoxyphenyl)-N-(tert-butoxycarbonyl)amino]phenylcarbamate

0.67 g (4.95 mmol) of 1-hydroxybenzotriazole, 1.28 g (9.9 mmol) of N,N-diisopropylethylamine and then 0.452 g (1.65 mmol) of the carbonate prepared in Example 256a are added to a solution composed of 0.52 g (1.65 mmol) of 4-amino4′-methoxy-N-butoxycarbonyldiphenylamine in a mixture of 5 ml of DMF and 5 ml of dichloromethane. The reaction medium is stirred for 27 hours at room temperature and then taken up in 30 ml of 1N NaOH solution and extracted with CH₂Cl₂. The organic phase is washed with saturated NaCl solution, dried over Na₂SO₄ and concentrated to give 0.99 g of a brown oil, which is purified by flash chromatography on a column of silica in a 4:1 to 1:1 heptane/ethyl acetate gradient. 0.29 g of a beige-coloured solid is obtained.

(Yield=39.2%).

M.p.=118° C.

NMR (DMSO-d6)=1.4 (9H, s); 3.7 (3H, s); 5.2 (2H, s); 6.9 (2H, d, J=8.8 Hz); 7.2 (4H, d, J=8.8 Hz); 7.45 (3H, m); 7.9 (1H, m); 8.6 (1H, d, J=4.4 Hz); 8.7 (1H, s); 9.9 (1H, s).

c) Pyrid-3-ylmethyl 4-[N-(4-methoxyphenyl)amino]phenylcarbamate

Obtained by performing the process as in Example 253b, using a 15% solution of trifluoroacetic acid in CH₂Cl₂.

(Yield=61.6%)

NMR (DMSO-d6)=3.7 (3H, s); 5.2 (2H, s); 6.8-7.05 (6H, m); 7.25-7.35 (2H, d, J=8.8 Hz); 7.45 (1H, m); 7.6 (1H, s); 7.9 (1H, d, J=7.9 Hz); 8.6 (1H, m); 8.7 (1H, s); 9.6 (1H, broad s).

d) Pyrid-3-ylmethyl 4-[1-(4-methoxyphenyl)-2-oxohydrazino]phenylcarbamate

Obtained by performing the process as in Example 1c.

(Qualitative Yield)

NMR (DMSO-d6)=3.75 (3H, 2 s); 5.2 (2H, 2s); 6.9 -7.1 (4H, m); 7.2-7.5 (3H, m) 7.5-7.65 (2H, m); 7.8-7.9 (1H, d, J=7.4 Hz); 8.5 (1H, m); 8.65 (1H, s); 10.0 (1H, 2s).

### EXAMPLES 257-265

Other examples of carbamates:

| | NMR |
|---|---|
| Compounds of the formula 2 | |
| 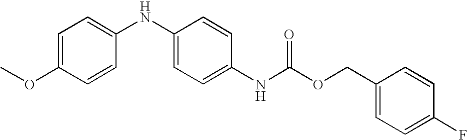 | (DMSO-d6): 3.6 (3H, s); 5.0 (2H, s); 6.7-6.9 (6H, m); 7.1-7.3 (4H, m); 7.35-7.5 (2H, m); 7.6 (1H, s); 9.4 (1H, broad s) |
| 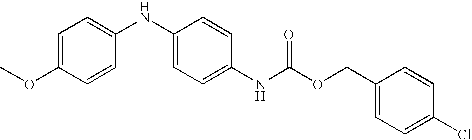 | (DMSO-d6): 3.6 (3H, s); 5.1 (2H, s); 6.7-7.0 (6H, m); 7.15-7.3 (2H, m); 7.4 (4H, m); 7.6 (1H, m); 9.4 (1H, s large) |
| 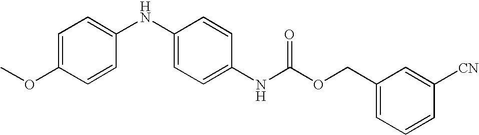 | (DMSO-d6): 3.55 (3H, s) 5.1 (2H, s); 6.7-6.95 (6H, m); 7.1-7.3 (2H, m); 7.35-7.9 (5H, m); 9.45 (1H, s large) |
| 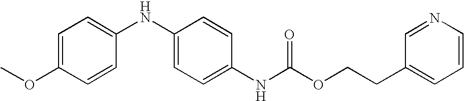 | (DMSO-d6): 2.9 (2H, t, J=6.5 Hz); 3.65 (3H, s); 4.25 (2H, t, J=6.5 Hz); 6.75-6.95 (6H, m); 7.15-7.4 (3H, m); 7.6-7.75 (2H, m); 8.4 (1H, d, J=4.4 Hz); 8.5 (1H, s); 9.25 (1H, s large) |
| 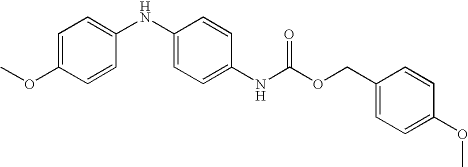 | |
| 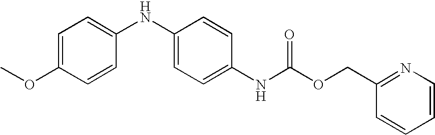 | |
| 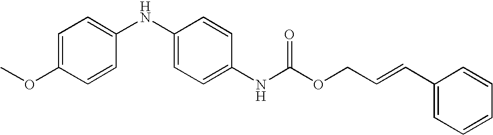 | |
| 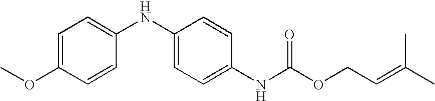 | |
| 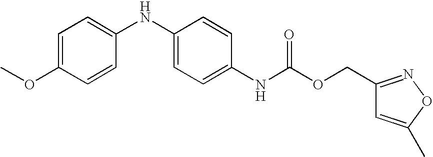 | |
| Compounds of the formula 1 | |
| 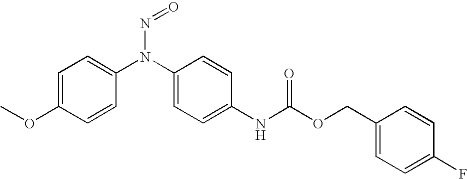 | (DMSO-d6): 3.7 (3H, s); 5.1 (2H, s); 6.95-7.1 (4H, m); 7.1-7.25 (2H, m); 7.25-7.35 (2H, m); 7.4-7.6 (4H, m); 9.95 (1H, 2s) |
| 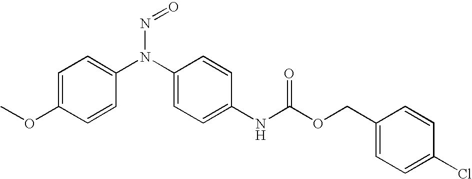 | (DMSO-d6): 3.75 (3H, 2s); 5.1 (2H, 2s); 7.02 (4H, m); 7.27 (2H, d, J=8.9 Hz); 7.4 (4H, s); 7.5 (2H, m); 10.0 (1H, 2s) |
| 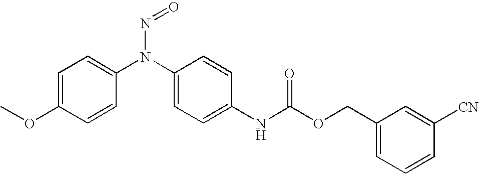 | (DMSO-d6): 3.7 (3H, 2s); 5.15 (2H, 2s); 6.9-7.1 (4H, m); 7.2-7.3 (2H, m); 7.45-7.6 (3H, m); 7.65-7.80 (2H, m); 7.8 (1H, s); 10.0 (1H, 2s) |
| 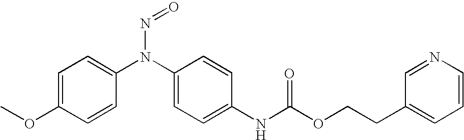 | (DMSO-d6): 2.9 (2H, t, J=6.5 Hz); 3.75 (3H, 2s); 4.35 (2H, t, J=6.5 Hz); 7.0-7.2 (4H, m); 7.25-7.4 (3H, m); 7.55 (2H, t, J=9 Hz); 7.7 (1H, d, J=7.8 Hz); 8.4 (1H, d, J=4.5 Hz); 8.5 (1H, s); 9.85 (1H, 2s) |
| 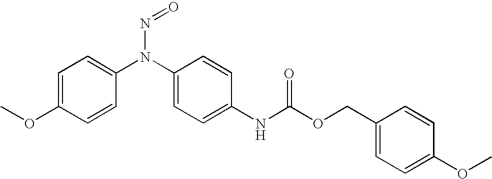 | |
| 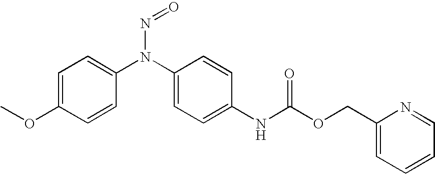 | |
| 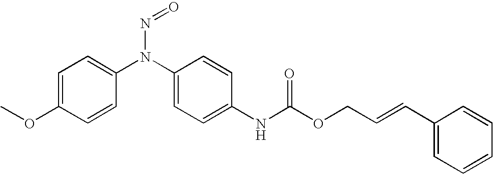 | |
| 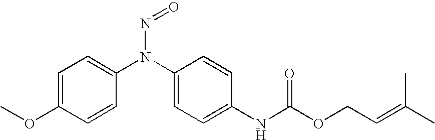 | |
| 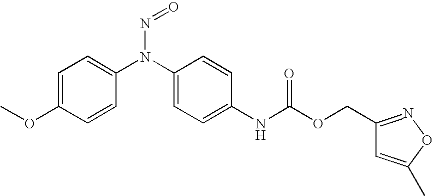 | |

### EXAMPLE 266

NMR and/or MS characterization of a number of compounds of the formula II

| T | H | A | Y | NMR and/or MS |
|---|---|---|---|---|
| H | H | CO | 3,5-di-tBu-4-OH-Ph-CH2 | (CDCl3): 1.3 (18H, s); |
| | | | | 3.5 (2H, s); 4.2 (1H, |
| | | | | broad s, exchangeable |
| | | | | with CF3COOD); 5.1 (1H, |
| | | | | s, exchangeable with |
| | | | | CF3COOD) ; 6.6-7.2 |
| | | | | (12H, m) |
| 4-MeO | H | CO | 1-Et-3-Me-pyrazol-5-yl | (CDCl3): 1.45 (3H, t, J= |
| | | | | 7.1 Hz); 2.3 (3H, s); 3.8 |
| | | | | (3H, s); 4.45 (2H, q, J= |
| | | | | 7.1 Hz); 5.6 (1H, broad |
| | | | | s); 6.4 (1H, s); 6.9-7.05 |
| | | | | (4H, m); 7.1 (2H, m); 7.4- |
| | | | | 7.5 (2H, d, J 32 8.8 Hz) |
| | | | | 7.6 (1H, broad s) |
| H | H | CO | 3-tBu-4-OH-Ph-CH2 | (CDCl3): 1.3 (9H, s); 3.5 |
| | | | | (2H, s); 5.1 (1H, s); 5.5 |
| | | | | (1H, broad s); 6.6 (1H, d, |
| | | | | J=8.0 Hz); 6.8-7.0 (3H, |
| | | | | m); 7.1-7.3 (9H, m) |
| 4-MeO | H | SO2 | 3-pyridyl | (DMSO-d6): 3.6 (3H, s); |
| | | | | 6.7-6.9 (6H, m); 6.9-7.0 |
| | | | | (2H, m); 7.5 (1H, m) |
| | | | | 8.05 (1H, m); 8.7 (3H, |
| | | | | m); 9.9 (1H, s) |
| 4-MeO | H | CO | 1-oxide-3-pyridyl | (DMSO-d6): 4.0 (3H, s); |
| | | | | 7.0-7.4 (6H, m); 7.7-8.2 |
| | | | | (5H, m); 8.6 (1H, d, J= |
| | | | | 6.4H z); 8.9 (1H, s); 10.5 |
| | | | | (1H, s) |
| 4-MeO | H | CO | 3-NO2-Ph | (DMSO-d6): 3.9 (3H, s); |
| | | | | 7.0-7.35 (6H, m); 7.7-7.8 |
| | | | | (2H, d, J=8.9 Hz); 8.0 |
| | | | | (2H, m); 8.6 (2H, m); 8.9 |
| | | | | (1H, s); 10.6 (1H, s) |
| 4-MeS | H | CO | Ph | (DMSO-d6): 2.5 (3H, s); |
| | | | | 7.0-7.15 (4H, m); 7.2-7.3 |
| | | | | (2H, m); 7.5-7.75 (5H, |
| | | | | m); 7.9-8.0 (2H, m); 8.2 |
| | | | | (1H, s); 10.1 (1H, s) |
| H | H | SO2 | 2-thienyl | (CDCl3) 6.8-7.3 (12H, |
| | | | | m); 7.4-7.55 (2H, m) |
| | | | | LC-MS: ES+: 330.88 |
| | | | | (M + 1) ES−: 328.86 (M − 1) |
| H | H | SO2 | 3-F-Ph | LC-MS: ES+: 342.84 |
| | | | | (M + 1) ES−: 340.86 (M − 1) |
| H | H | SO2 | 4-CN-Ph | LC-MS: ES+: 349.83 |
| | | | | (M + 1) ES−: 347.87 (M − 1) |
| H | H | SO2 | Ph-CH = CH— | (CDCl3): 6.7 (1H, d, J= |
| | | | | 15.5 Hz); 6.8-7.1 (8H, |
| | | | | m); 7.15-7.35 (8H, m); |
| | | | | 7.6 (1H, broad s) LC-MS: |
| | | | | ES+: 350.89 (M + 1) ES−: |
| | | | | 348.89 (M − 1) |
| H | H | SO2 | 4-Ac-NH-Ph | LC-MS: ES+: 382.19 |
| | | | | (M + 1) |
| H | H | SO2 | 3-Cl-4-Ac-NH-Ph | (CDCl3): 2.45 (3H, s); |
| | | | | 7.1-7.35 (8H, m); 7.4-7.6 |
| | | | | (3H, m); 7.85 (1H, m) |
| | | | | 7.9 (1H, m); 8.1 (2H, 2 |
| | | | | broad s) LC-MS: ES−: |
| | | | | 413.83 (M − 1) |
| H | H | SO2 | 4-NO2-Ph | LC-MS: ES+: 369.77 |
| | | | | (M + 1) ES−: 367.88 (M − 1) |
| 4-MeO | H | SO2 | 4-MeO-Ph | LC-MS: ES+: 384.89 |
| | | | | (M + 1) ES−: 382.85 (M − 1) |
| 4-MeO | H | CO | 2-furyl | LC-MS: ES+: 309.15 |
| | | | | (M + 1) |
| 4-MeO | H | CO | 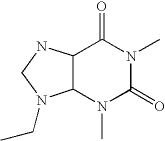 | LC-MS: ES+: 435.39 (M + 1) |
| 4-MeO | H | CO | 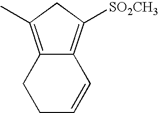 | LC-MS: ES+: 455.35 (M + 1) ES−: 453.35 (M − 1) |
| 4-MeO | H | CO | 3-CF3-Ph | LC-MS: ES+: 387.19 |
| | | | | (M + 1) ES−: 385.21 (M − 1) |
| 4-MeO | H | CO | 2-NO2-Ph-CH = CH— | LC-MS: ES+: 390.18 |
| | | | | (M + 1) ES−: 388.23 (M − 1) |
| 4-MeO | H | CO | 2-(4-MeOPhO)-5-NO2Ph | LC-MS: ES+: 486.23 |
| | | | | (M + 1) ES−: 484.25 (M − 1) |
| 4-MeO | H | CO | 3.5-di-tBu-4-OH-Ph- | LC-MS: ES+: 447.37 |
| | | | | (M + 1) ES−: 445.36 (M − 1) |
| 4-MeO | H | CO | 7-MeO-coumar-3-yl | LC-MS: ES+: 417.24 |
| | | | | (M + 1) |
| 4-MeO | H | CO | 5-Hex-2-thienyl | LC-MS: ES+: 409.27 |
| | | | | (M + 1) |
| 4-MeO | H | CO | 2.5-diCl-pyrid-3-yl | (acetone-d6): 3.6 (3H, s); |
| | | | | 6.6-7.0 (7H, m); 7.4 (2H, |
| | | | | m); 8.0 (1H, s); 8.4 (1H, |
| | | | | s); 9.4 (1H, broad s) |
| 4-MeO | H | SO2 | 8-quinolyl | (acetone-d6): 3.55 (3H, |
| | | | | s); 6.4-6.9 (9H, m); 7.5- |
| | | | | 7.7 (2H, m); 8.0-8.15 (2H, |
| | | | | m); 8.4 (1H, m); 8.6 (1H, |
| | | | | s); 9.0 (1H, broad s) |

1. Compound of the formula I: 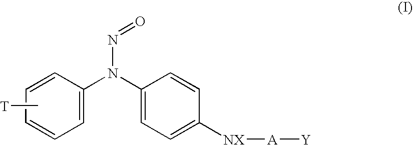
in which:
X represents a hydrogen atom; a saturated or unsaturated aliphatic hydrocarbon-based radical; or a group -A—Y;
A represents —CO—; SO₂—; —CO—NRₐ- in which the carbonyl group is linked to the nitrogen atom of NX and Rₐ represents a hydrogen atom or a saturated or unsaturated aliphatic hydrocarbon-based radical; or —CO—NRₐ—SO₂— in which the carbonyl group is linked to the nitrogen atom of NX and Rₐ is as defined above;
T represents a hydrogen atom; a halogen atom; a saturated or unsaturated aliphatic hydrocarbon-based group, optionally interrupted with O and/or S and optionally halogenated; nitro; or cyano;
Y may represent any organic substituent when A represents —CO—, and, in the general case, Y is chosen from:
a saturated or unsaturated, aliphatic hydrocarbon-based radical optionally interrupted with O and/or S;
a radical of the formula —(O)ₘ-(alk″)ₙ-Rcy in which m represents 0 or 1; when m represents 0, then n represents 0 and when m represents 1, then n is chosen from 0 and 1; alk″ represents nothing or represents a saturated aliphatic hydrocarbon-based chain; and Rcy represents (i) either a saturated, unsaturated and/or aromatic carbocyclic radical, optionally substituted with one or more substituents chosen from oxo and the radicals R defined below; (ii) or a saturated, unsaturated and/or aromatic heterocyclic radical, optionally substituted with one or more substituents chosen from oxo and the radicals R defined below; it being understood that when A represents CO—NRa, then m represents 0;
a radical 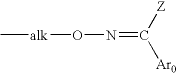
in which alk and Z independently represent a saturated or unsaturated aliphatic hydrocarbon-based chain, Z also possibly representing a hydrogen atom, and Ar₀ represents a saturated, unsaturated and/or aromatic, carbocyclic radical optionally substituted with one or more substituents chosen from oxo and the radicals R defined below, or alternatively Ar₀ represents a saturated, unsaturated and/or aromatic heterocyclic radical optionally substituted with one or more substituents chosen from oxo and the radicals R defined below;
a radical -alk′—W—Cy in which alk′ is as defined above for alk, except that it may also be substituted with one or more radicals G as defined below; W is chosen from O, S, —NH—SO₂—, —NH—CO—, —CO—NH—, —CO— and —SO₂; and Cy represents a saturated or unsaturated aliphatic hydrocarbon-based radical, optionally substituted with one or more radicals G as defined below; or alternatively Cy represents a saturated, unsaturated and/or aromatic carbocyclic radical optionally substituted with one or more substituents chosen from oxo and the radicals R defined below; or alternatively Cy represents a saturated, unsaturated and/or aromatic heterocyclic radical optionally substituted with one or more substituents chosen from oxo and the radicals R defined below; it being understood that when alk′ and Cy do not both represent an unsubstituted saturated or unsaturated aliphatic hydrocarbon-based radical, then W can represent nothing, in which case Cy may also represent one of the radicals R defined below; and
a radical -(alk-NH—CO)_{q}-Ar₀ in which alk and Ar₀ are as defined above; and q represents an integer from 1 to 5;
G represents a halogen atom; a cyano group; a nitro group; a hydroxyl group; an amino group; an alkylamino group; a dialkylamino group; an aryl group which is optionally halogenated and/or optionally substituted with alkyl; an alkyl group which is optionally interrupted with O and/or S and optionally halogenated;
R is chosen from a halogen atom; a cyano group; a nitro group; an amino group; an alkylamino group; a dialkylamino group; a dialkylaminoalkoxy group; a dialkylaminoalkylthio group; an aryl group optionally substituted with one or more radicals G; an alkyl group optionally interrupted with O and/or S and optionally halogenated; a hydroxyl group; an alkylthio group substituted with arylsulfonyl in which aryl is optionally substituted with one or more radicals G; an aryloxy group in which aryl is optionally substituted with one or more radicals G; an arylthio group in which aryl is optionally substituted with one or more radicals G; an alkylsulfonyl group; an arylsulfonyl group in which aryl is optionally substituted with one or more radicals G; an alkylcarbonyl group; a heteroaryl group comprising one or more hetero atoms chosen from O, N and S and optionally substituted with one or more radicals G and/or with alkoxycarbonyi; an alkoxycarbonyl group; an alkylcarbonyloxy group; an alkylcarbonylamino group; an alkylenedioxy group; an alkylene group optionally substituted with oxo; an arylalkyl group in which aryl is optionally substituted with one or more radicals G; a cycloalkyl group optionally substituted with one or more radicals G; a cycloalkylalkyl group in which cycloalkyl is optionally substituted with one or more radicals G and/or with arylsulfonylamino in which aryl is itself optionally halogenated;
the stereoisomers thereof, the addition salts thereof with acids or bases and the hydrates and solvates thereof.

2. Compound of the formula I according to claim 1, characterized in that X represents H; (C₁—C₁₄)alkyl; or the group A-Y in which A and Y are as defined in claim 1.

3. Compound of the formula I according to either of claims 1 and 2, characterized in that A represents CO; SO₂; —CO—NRₐ in which Rₐ represents H or (C₁—C₁₄)alkyl; or alternatively A represents —CO—NRa—SO₂— in which the carbonyl group is linked to the nitrogen atom of —NX in which Ra is H or (C₁—C₁₄)alkyl.

4. Compound of the formula I according to any one of claims 1 to 3, characterized in that T is H; optionally halogenated (C₁—C₁₄)alkoxy; or optionally halogenated (C₁—C₁₄)thioalkoxy.

5. Compound of the formula I according to any one of claims 1 to 4, characterized in that Y is chosen from:
a) (C₁—C₁₀)alkyl;
b) (C₁—C₁₀)alkoxy-(C₁—C₁₀)alkyl;
c) (C₁—C₁₀)alkoxy-(C₁—C₁₀)alkoxy;
d) coumarinyl optionally substituted with one or more radicals G as defined in claim 1;
e) a group 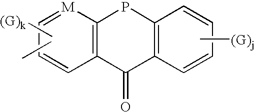
in which j and k independently represent an integer from 0 to 4;
M represents N or C;
P represents SO₂ or O;
G is as defined in claim 1;
f) a group 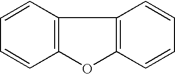
optionally substituted with one or more radicals G as defined in claim 1;
g) a group 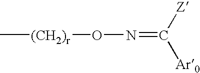
in which r is an integer from 0 to 6;
Z′ represents a hydrogen atom or (C₁—C₁₀)alkyl;
Ar′ₒ represents (C₆—C₁₀)aryl optionally substituted with one or more radicals G as defined in claim 1;
h) a group -alks-W′—Cy′ in which:
alks represents (C₁—C₁₀)alkylene optionally substituted with (C₆—C₁₀)aryl, itself optionally substituted with one or more radicals G as defined above;
W′ represents O, S, —NH—SO₂—, —NH—CO—, —CO—NH—, —CO— or —SO₂—;
Cy′ represents (C₁—C₁₄)alkyl optionally substituted with (C₆—C₁₀)aryl and/or amino; (C₆—C₁₀)aryl optionally substituted with one or more radicals G as defined above; 5- to 7-membered heteroaryl comprising one or more hetero atoms chosen from O, N and S, optionally substituted with one or more radicals G as defined above; or a saturated 5- to 7-membered heterocycle comprising one or more hetero atoms chosen from O, N and S, optionally substituted with one or more radicals G as defined above and/or with an oxo group;
i) a group -(alks′-NH—CO)_{q}-(C₆—C₁₀)aryl in which alks′ represents (C₁—C₆)alkylene; q represents an integer from 1 to 5; and aryl is optionally substituted with one or more radicals G as defined in claim 1;
j) (C₂—C₁₀)alkenyl optionally substituted with a group —NH—CO—(C₁—C₁₀)alkyl; with a group (C₆—C₁₀)aryl itself optionally substituted with one or more radicals G as defined in claim 1; with a 5- to 7-membered heteroaryl group comprising one or more hetero atoms chosen from O, N and S, itself optionally substituted with one or more radicals G as defined in claim 1; and/or with a group —CO—NH—(C₁—C₁₀)alkyl;
k) -(alk″)ₚ-Ar′ in which
p represents the integer 0 or 1;
alk″ represents (C₁—C₆)alkylene or (C₂—C₆)alkenylene;
Ar′ represents (C₃—C₈)cycloalkyl optionally substituted with one or more radicals G as defined in claim 1 and/or with oxo, and optionally fused to (C₆—C₁₀)aryl, the said aryl nucleus optionally being substituted with one or more radicals Gas defined in claim 1;
or alternatively Ar′ represents heteroaryl with a monocyclic, bicyclic or tricyclic nucleus comprising one or more hetero atoms chosen from O, N and S, the said hetero atoms N and S optionally being in oxidized form, in which each ring of the said monocyclic, bicyclic or tricyclic nucleus is 5- to 7-membered, the rings not directly linked to the group —NX—A-(alk″)p- optionally being partially or totally hydrogenated, the said heteroaryl optionally being substituted with one or more radicals R as defined in claim 1 and/or where appropriate with an oxo group, it being understood that heteroaryl also denotes the mesomeric forms of the mono-, bi- and tricyclic nuclei defined in claim 1;
or alternatively Ar′ represents 5- to 7-membered saturated or unsaturated heterocycle comprising one or more hetero atoms chosen from N, O and S and optionally substituted with one or more oxo radicals and/or radicals G as defined in claim 1, the nitrogen atom also possibly being optionally substituted with (C₁—C₆)alkylcarbonyl; with (C₆—C₁₀)arylsulfonyl; or with (C₆—C₁₀)aryl-(C₁—C₆)alkyl, in which the aryl portions are optionally substituted with one or more radicals G as defined in claim 1; the said heterocycle optionally being fused to a (C₆—C₁₀)aryl nucleus optionally substituted with one or more radicals G as defined in claim 1;
or alternatively Ar′ represents (C₆—C₁₀)aryl optionally substituted with one or more radicals R as defined below, or, when p is other than 0, aryl is optionally substituted with (C₃—C₈)cycloalkyl-(C₁—C₆)alkyl in which cycloalkyl is itself substituted with (C₆—C₁₀)arylsulfonylamino in which aryl is optionally halogenated.

6. Compound according to claim 5, characterized in that A represents CO.

7. Compound according to any one of claims 1 to 6, characterized in that G is chosen from halogen; hydroxyl; optionally halogenated (C₁—C₁₄)alkoxy; optionally halogenated (C₁—C₁₄)alkyl; nitro; cyano; amino; (C₁—C₁₄)alkylamino; di(C₁—C₁₄)alkylamino; (C₆—C₁₀)aryl which is optionally halogenated and/or optionally substituted with (C₁—C₁₄)alkyl.

8. Compound according to any one of claims 1 to 7, characterized in that R is chosen from a halogen atom; cyano; hydroxyl; nitro; optionally halogenated (C₁—C₁₀)alkyl; optionally halogenated (C₁—C₁₀) alkoxy; (C₁—C₁₀)alkylthio optionally substituted with (C₆—C₁₀)arylsulfonyl in which aryl is optionally substituted with one or more radicals G; (C₆—C₁₀)aryloxy in which aryl is optionally substituted with one or more radicals G; (C₆—C₁₀)arylthio in which aryl is optionally substituted with one or more radicals G; (C₁—C₁₀)alkylsulfonyl; (C₆—C₁₀)arylsulfonyl in which aryl is optionally substituted with one or more radicals G; 5- to 7-membered heteroaryl comprising one or more hetero atoms chosen from O, N and S and optionally substituted with one or more radicals G and/or with (C₁—C₁₀)alkoxycarbonyl; (C₁—C₁₀)alkoxycarbonyl; (C₁—C₁₀)alkylcarbonylamino; di(C₁—C₁₀)alkylamino; (C₂—C₄)alkylenedioxy; (C₃—C₅)alkylene optionally substituted with oxo; (C₆—C₁₀)aryl-(C₁—C₁₀)alkyl in which aryl is optionally substituted with one or more radicals G; amino; (C₁—C₁₀)alkylamino; di(C₁—C₁₀)alkylamino; optionally halogenated (C₆—C₁₀)aryl; (C₁—C₁₀)alkylcarbonyl; (C₃—C₈)cycloalkyl-(C₁—C₆)alkyl in which cycloalkyl is itself substituted with (C₆—C₁₀)arylsulfonylamino in which aryl is optionally halogenated;
G being as defined in claim 1.

9. Compound according to any one of claims 5 to 8, characterized in that G represents halogen; optionally halogenated (C₁—C₆)alkyl; optionally halogenated (C₁—C₆)alkoxy; nitro or cyano.

10. Compound according to any one of claims 5 to 9, characterized in that R is chosen from R is chosen from a halogen atom; cyano; hydroxyl; nitro; optionally halogenated (C₁—C₁₀)alkyl; optionally halogenated (C₁—C₁₀) alkoxy; (C₁—C₁₀)alkylthio optionally substituted with (C₆—C₁₀)arylsulfonyl in which aryl is optionally substituted with one or more radicals G; (C₆—C₁₀)aryloxy in which aryl is optionally substituted with one or more radicals G; (C₆—C₁₀)arylthio in which aryl is optionally substituted with one or more radicals G; (C₁—C₁₀)alkylsulfonyl; (C₆—C₁₀)arylsulfonyl in which aryl is optionally substituted with one or more radicals G; 5- to 7-membered heteroaryl comprising one or more hetero atoms chosen from O, N and S and optionally substituted with one or more radicals G and/or with (C₁—C₁₀)alkoxycarbonyl; (C₁—C₁₀)alkoxycarbonyl; (C₁—C₁₀)alkylcarbonylamino; di(C₁—C₁₀)alkylamino; (C₂—C₄)alkylenedioxy; (C₃—C₅)alkylene optionally substituted with oxo; and (C₆—C₁₀)aryl-(C₁—C₁₀)alkyl in which aryl is optionally substituted with one or more radicals G.

11. Compound according to any one of claims 5 to 10, characterized in that Y is chosen from:
-alks-He—Cy′ in which alks represents (C₁—C₆)alkylene, He represents O or S, and Cy′ represents (C₆—C₁₀)aryl optionally substituted with one or more radicals G; heteroaryl optionally substituted with one or more radicals G; or (C₁—C₁₄)alkyl;
-alks-NH—SO₂—Cy′ in which alks represents (C₁—C₁₀)alkylene; Cy′ represents heteroaryl optionally substituted with one or more radicals G;
-alks-NH—CO—Cy′ in which alks represents (C₁—C₆)alkylene; Cy′ represents (C₁—C₁₄)alkyl; (C₆—C₁₀)aryl optionally substituted with one or more radicals G; saturated heteroaryl optionally substituted with one or more radicals G; saturated heterocycle optionally substituted with one or more radicals G and/or oxo; or (C₁—C₆)alkyl optionally substituted with amino and/or (C₆—C₁₀)aryl;
-alks-CO—NH—Cy′ in which alks represents (C₁—C₆)alkylene; Cy′ represents phenyl optionally substituted with one or more radicals G;
-alks-CO—Cy′ in which alks represents (C₁—C₁₀)alkylene; Cy′ represents heteroaryl optionally substituted with one or more radicals G;
-alks-SO₂—Cy′ in which alks represents (C₁—C₆)alkylene; Cy′ represents (C₆—C₁₀)aryl optionally substituted with one or more radicals G;
G being as defined in claim 1.

12. Compound according to any one of claims 5 to 10, characterized in that Y represents -(alk″)p-Ar′ in which alk″ comprises from 1 to 3 carbon atoms, p is 1 and Ar′ represents:
(C₃—C₈)cycloalkyl optionally substituted with oxo and optionally fused to a phenyl nucleus which is itself optionally substituted with one or more radicals G;
5- to 7-membered heteroaryl optionally substituted with one or more radicals G;
phenyl optionally substituted with one or more radicals G and/or with (C₃—C₈)cycloalkyl-(C₁—C₆)alkyl in which cycloalkyl is itself substituted with (C₆—C₁₀)arylsulfonylamino in which aryl is optionally halogenated;
or a 5- to 7-membered heterocyclic radical comprising one or two hetero atoms chosen from O, N and S and fused to a phenyl nucleus, the said radical optionally being substituted with one or more radicals G;
G being as defined in claim 1.

13. Compound according to any one of claims 5 to 10, characterized in that Y represents Ar′ in which Ar′ is chosen from:
(C₃—C₈)cycloalkyl optionally fused to phenyl and optionally substituted with one or more oxo radicals and/or radicals G, the phenyl nucleus itself optionally being substituted with one or more radicals G;
phenyl optionally substituted with one or more radicals R (R preferably being chosen from alkoxy; halogen; nitro; alkoxycarbonyl; alkylcarbonylamino; hydroxyl; optionally halogenated alkyl; alkylsulfonyl; 5- to 7-membered heteroaryl optionally substituted with one or more radicals G, for example optionally substituted pyrazolyl; alkylenedioxy);
5- to 7-membered heteroaryl optionally substituted with one or more radicals R (R preferably being chosen from thioalkoxy optionally substituted with phenylsulfonyl in which phenyl is itself substituted with one or more radicals G; phenyloxy optionally substituted with one or more radicals G; optionally halogenated alkyl; halogen; alkylsulfonyl; NO₂; optionally halogenated alkoxy;
5- to 7-membered heteroaryl optionally substituted with alkoxycarbonyl and/or with one or more radicals G; phenylthio optionally substituted with one or more radicals G);
saturated and/or unsaturated 5- to 7-membered heterocycle optionally fused to a phenyl nucleus, the whole optionally being substituted with one or more radicals R and/or oxo, R preferably being chosen from alkylcarbonyl; phenylalkyl; phenylsulfonyl in which phenyl is optionally substituted with one or more radicals G; alkoxy;
bicyclic heteroaryl in which each monocycle is 5 to 7-membered, the monocycle not directly linked to —NX-A- optionally being partially hydrogenated, the said radical optionally being substituted with one or more radicals R and/or oxo, R preferably being chosen from nitro, alkyl, alkylsulfonyl and alkoxy.

14. Compound according to any one of claims 5 to 10, characterized in that A represents SO₂; CO—NRa; or CO—NRa—SO₂; and Y represents (C₁—C₁₀) alkyl optionally substituted with (C₁—C₁₀)alkylsulfonyl; (C₃—C₈)cycloalkyl; or alternatively -(alk′)_{q}-Ar″
in which
q is the integer 0 or 1,
alk″ represents (C₁—C₆)alkylene or (C₂—C₆)alkenylene, and
Ar″ represents (C₆—C₁₀)aryl optionally substituted with one or more radicals R as defined in claim 1;
or alternatively Ar″ represents heteroaryl with a monocyclic, bicyclic or tricyclic nucleus comprising one or more hetero atoms chosen from O, N and S, the hetero atoms N and S optionally being in oxidized form, each ring of the said monocyclic, bicyclic or tricyclic nucleus being 5- to 7-membered, and the said heteroaryl optionally being substituted with one or more radicals R as defined in claim 1.

15. Compound according to claim 14, characterized in that:
R is chosen from halogen; optionally halogenated (C₁—C₁₀)alkyl; optionally halogenated (C₁—C₁₀)alkoxy; nitro; (C₁—C₁₀)alkoxycarbonyl; (C₁—C₁₀)alkylcarbonyl; (C₁—C₁₀)alkylcarbonylamino; di(C₁—C₁₀)alkylamino; cyano; (C₁—C₁₀)alkylthio; (C₆—C₁₀)aryloxy in which aryl is optionally substituted with one or more radicals G as defined in claim 1; (C₁—C₁₀)alkylsulfonyl; (C₆—C₁₀)arylsulfonyl optionally substituted with one or more radicals G as defined in claim 1; and 5- to 7-membered heteroaryl comprising one or more hetero atoms chosen from O, N and S and optionally substituted with one or more radicals G as defined in claim 1 and in which the nitrogen and sulfur atoms are optionally in oxidized form;
G is chosen from halogen; (C₁—C₆)alkyl; (C₁—C₆)alkoxy; nitro or cyano;
T is chosen from H; (C₁—C₁₀)alkoxy; (C₁—C₁₀)alkylthio; or optionally halogenated (C₁—C₁₀)alkyl.

16. Compound according to claim 1, characterized in that:
A represents SO₂;
Y represents:
(C₁—C₆)alkyl;
phenyl optionally substituted with one or more halogen, nitro, cyano, optionally halogenated (C₁—C₆)alkyl, optionally halogenated (C₁—C₆)alkoxy, (C₁—C₆)alkylcarbonylamino, (C₁—C₆)alkylcarbonyl, (C₁—C₆)alkoxycarbonyl, di(C₁—C₆)alkylamino, (C₁—C₆)alkylsulfonyl, or phenoxy optionally substituted with one or more radicals G as defined in claim 1;
naphthyl optionally substituted with one or more di(C₁—C₆)alkylamino;
phenyl-(C₁—C₆)alkyl in which phenyl is optionally substituted with one or more radicals G as defined in claim 1;
(C₁—C₆)alkyl optionally substituted with (C₁—C₆)alkylsulfonyl;
(C₃—C₈)cycloalkyl;
phenyl-(C₂—C₆)alkenyl in which phenyl is optionally substituted with one or more radicals G as defined in claim 1;
5- to 7-membered monocyclic heteroaryl chosen from imidazolyl, pyrazolyl, thiazolyl, thienyl, pyridyl, pyrazolyl, furyl, N-oxypyridyl, pyrazinyl, pyrimidinyl and isoxazolyl, the said heteroaryl optionally being substituted with one or more radicals chosen from (C₁—C₆)alkoxy, (C₁—C₆)alkylthio, halogen, (C₁—C₆)alkyl, di(C₁—C₆)alkylamino, (C₁—C₆)alkylcarbonylamino, (C₁—C₆)alkoxycarbonyl, phenyl-sulfonyl and pyridyl;
bicyclic heteroaryl chosen from quinolyl, isoquinolyl, benzothienyl and a radical of the formula: 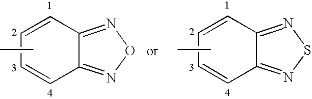
the said bicyclic heteroaryl optionally being substituted with one or more radicals G as defined in claim 1;
or heteroaryl-(C₁—C₆)alkyl in which heteroaryl represents 5- to 7-membered monocyclic heteroaryl as defined in claim 1, the said heteroaryl optionally being substituted with one or more radicals G as defined in claim 1.

17. Compound according to claim 16, characterized in that A represents SO₂; Y represents quinolyl optionally substituted with one or more radicals G; optionally substituted pyridyl; optionally substituted pyrimidinyl.

18. Compound according to claim 16, characterized in that X represents H; A represents SO₂; Y represents phenyl optionally substituted with one or more radicals chosen from nitro, halogen, optionally halogenated (C₁—C₆)alkyl, and optionally halogenated (C₁—C₆)alkoxy; pyridyl optionally substituted with one or more radicals chosen from (C₁—C₆)alkoxy, halogen and (C₁—C₆)alkyl; T represents a hydrogen atom or (C₁—C₆)alkoxy.

19. Compound according to claim 1 or 2, characterized in that A represents —CO—NRa— or —CO—NRa—SO₂—; Y represents phenyl optionally substituted with one or more halogen, nitro, cyano, optionally halogenated (C₁—C₆)alkyl, optionally halogenated (C₁—C₆)alkoxy, (C₁—C₆)alkylcarbonylamino, (C₁—C₆)alkylcarbonyl, (C₁—C₆)alkoxycarbonyl, di(C₁—C₆)alkylamino or phenoxy optionally substituted with one or more radicals G as defined in claim 1.

20. Compound according to any one of claims 1 to 10, characterized in that X represents —A—Y; and Y represents phenyl optionally substituted with one or more halogen, nitro, cyano, optionally halogenated (C₁—C₆)alkyl, optionally halogenated (C₁—C₆)alkoxy, (C₁—C₆)alkylcarbonylamino, (C₁—C₆)alkylcarbonyl, (C₁—C₆)alkoxycarbonyl, di(C₁—C₆)alkylamino, (C₁—C₆)alkylsulfonyl or phenoxy optionally substituted with one or more radicals G as defined in claim 1;
5- to 7-membered monocyclic heteroaryl chosen from imidazolyl, pyrazolyl, thiazolyl, thienyl, pyridyl, pyrazolyl, furyl, N-oxypyridyl, pyrazinyl, pyrimidinyl and isoxazolyl, the said heteroaryl optionally being substituted with one or more radicals chosen from (C₁—C₆)alkoxy, (C₁—C₆)alkylthio, halogen, (C₁—C₆)alkyl, di(C₁—C₆)alkylamino, (C₁—C₆)alkylcarbonylamino, (C₁—C₆)alkoxycarbonyl, phenylsulfonyl and pyridyl;
bicyclic heteroaryl chosen from quinolyl, benzothienyl and a radical chosen from 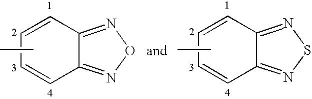
the said bicyclic heteroaryl optionally being substituted with one or more radicals G as defined in claim 1.

21. Compound according to any one of claims 1 to 10, characterized in that A represents CO; Y represents pyridyl, phenyl optionally substituted with one or more radicals G; or alkylphenyl optionally substituted with one or more radicals G.

22. Compound according to claim 1, characterized in that it corresponds to formula Ia: 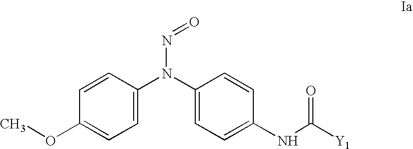
in which:
Y₁ is chosen from pyridyl optionally substituted with one or more substituents chosen from oxo and the radicals R defined above; pyrimidinyl optionally substituted with one or more substituents chosen from oxo and the radicals R defined above; and benzyl optionally substituted with one or more substituents chosen from oxo and the radicals R defined above.

23. Compound according to claim 1, characterized in that it corresponds to formula Ib: 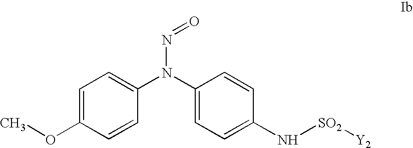
in which:
Y₂ represents 3-pyridyl optionally substituted with one or more substituents chosen from oxo and the radicals R defined above; phenyl optionally substituted with one or more substituents chosen from oxo and the radicals R defined above; >CH=CH—Cy^{o} in which Cy^{o} represents phenyl optionally substituted with one or more radicals G.

24. Compound of the formula II: 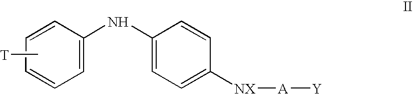
in which
T represents H;
A represents CO;
X is a hydrogen atom;
Y is chosen from:
benzyl optionally substituted on the phenyl nucleus with one or more radicals R with the exclusion of amino and nitro radicals, or alternatively Y represents —CH₂—Cy¹ in which Cy¹ is heteroaryl (with the exclusion of 2-pyridyl) optionally substituted with one or more radicals R as defined in claim 1;
phenyl substituted with one or more radicals chosen from nitro and optionally halogenated alkyl;
indolyl or pyrazinyl, indolyl and pyrazinyl optionally being substituted with one or more oxo radicals and/or radicals R as defined in claim 1.

25. Compound of the formula II: 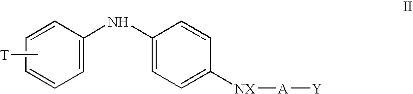
in which:
T represents H;
A represents SO₂;
X represents a hydrogen atom;
Y is chosen from a group —CH=CH—Cy⁰ in which Cy⁰ is phenyl optionally substituted with one or more radicals R; benzyl optionally substituted with one or more radicals R; heteroaryl (with the exclusion of benzopyran and coumarin) optionally substituted with one or more oxo radicals and/or radicals R; phenyl substituted with one or more radicals chosen from a fluorine atom, CF₃, OCF₃ and cyano; R being as defined in claim 1.

26. Compound of the formula II: 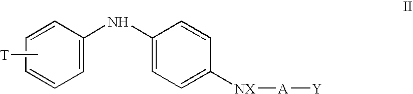
in which:
T represents methoxy;
X represents H;
A represents CO;
Y is as defined in claim 1 for formula I, except that Y does not take any of the following meanings: methyl; ethyl; ethoxy; 2-haloethyl; 2-mercapto-ethyl; vinyl; 1-methylvinyl; 3-amino-3-carboxypropyl; morpholinyl; phenyl; phenoxy; and benzyloxy.

27. Compound of the formula II: 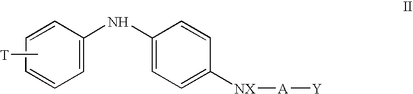
in which
T represents methoxy;
X represents H;
A represents SO₂;
Y is as defined for formula I in claim 1, on condition that Y does not represent unsubstituted phenyl.

28. Compound of the formula II: 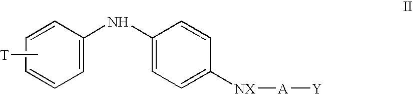
in which
T represents —CF₃ or alkylthio;
X represents H;
A and Y are as defined for formula I in claim 1.

29. Compound according to claim 28, characterized in that A represents CO or SO₂.

30. Compound of the formula II: 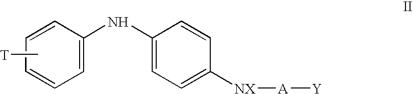
in which:
T represents methoxy;
X represents H;
Y represents pyridyl optionally substituted with one or more radicals R as defined in claim 1 for formula I.

31. Compound of the formula II: 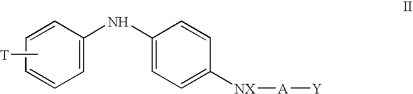
in which
T represents hydrogen;
A represents CO;
X represents —AY;
Y represents fury optionally substituted with one or more radicals R as defined in claim 1 for formula I.

32. Compound of the formula II: 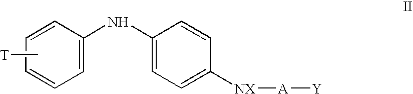
in which
T represents H;
A represents CONHSO₂;
X represents H;
Y represents phenyl optionally substituted with one or more radicals R as defined in claim 1 for formula I.

33. Compound of the formula II: 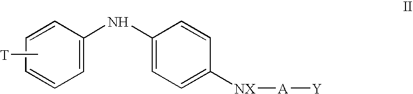
chosen from:
1) T=H; A=CO—NH—SO₂; Y=phenyl;
2) T=H; X=AY; A=CO; Y=cyclopentyl;
3) T=H; X=AY; A=SO₂; Y=phenyl;
4) T=4—OCH₃; X=CH₃; A=CO; Y-3-pyridyl;
5) T=H; A=CO; X=H; Y=2-methoxyphenyl;
6) T=H; A=CO; X=H; Y=2,4-dimethoxyphenyl;
7) T=H; A=CO; X=H; Y=2-pyridyl or 4-pyridyl or 2-furyl or 2,6-dimethoxy-3-pyridyl or 3-pyridyl N-oxide.

34. Process for preparing a compound of the formula I: 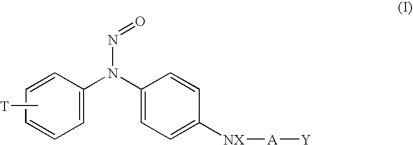
in which:
T, X, Y and A are as defined in claim 1, by nitrosation of a compound of the formula II: 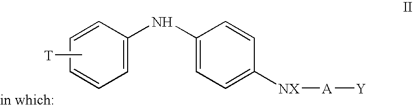
T, X, Y and A are as defined in claim 1, by the action of a suitable nitrosating agent.

35. Pharmaceutical composition comprising at least one compound of the formula I according to any one of claims 1 to 23, in combination with one or more pharmaceutically acceptable excipients.

36. Pharmaceutical composition comprising at least one compound of the formula II according to any one of claims 24 to 32, in combination with one or more pharmaceutically acceptable excipients.

37. Use of a compound of the formula I according to any one of claims 1 to 23, for the preparation of a medicinal product which may be used in the treatment of pathologies characterized by an oxidative stress condition and a lack of availability of endothelial nitrogen monoxide.

38. Use of a compound of the formula II according to any one of claims 24 to 32, for the preparation of an antioxidant medicinal product which may be used as a free-radical scavenger.

39. Use of a compound of the formula I according to any one of claims 1 to 23, for the preparation of a medicinal product which may be used in the treatment of
atherosclerosis-associated ischaemias (lipid peroxidation, development, progress and rupture of atheroma plaques, platelet activation);
restenosis after angioplasty;
stenosis after vascular surgery;
diabetes;
insulin resistance;
retinal and renal microvascular complications of diabetes;
the cardiovascular risk of diabetes in so far as it is not explained by the conventional factors;
male erectile dysfunction;
cerebral hypoxia;
chronic rejection after organ transplantation;
a articular pathologies.
